(19) United States
(12) Patent Application Publication
(10) Pub. No.: US 2023/0147495 A1
(43) Pub. Date: May 11, 2023

(54) GENOME ENGINEERING USING CRISPR RNA-GUIDED INTEGRASES

(71) Applicants: Phuc Hong VO, New York, NY (US); THE TRUSTEES OF COLUMBIA UNIVERSITY IN THE CITY OF NEW YORK, New York, NY (US)

(72) Inventors: Phuc Hong Vo, New York, NY (US); Samuel Sternberg, New York, NY (US)

(21) Appl. No.: 17/907,510

(22) PCT Filed: Mar. 26, 2021
(86) PCT No.: PCT/US21/24422

§ 371 Date: Sep. 27, 2022

Related U.S. Application Data
(60) Provisional application No. 63/001,008, filed on Mar. 27, 2020.
(60) Provisional application No. 63/053,460, filed on Jul. 17, 2020.
(60) Provisional application No. 63/081,677, filed on Sep. 22, 2020.

(51) Int. Cl. C12N 15/90; C12N 15/10; C12N 15/113; C12N 9/22

(52) U.S. Cl. CPC C12N 15/902; C12N 15/102; C12N 15/113; C12N 9/22; C12N 2310/20

(57) ABSTRACT

The present disclosure provides systems and methods for targeted nucleic acid deletions and inactivation of a gene of interest comprising a Clustered Regularly Interspaced Short Palindromic Repeats (CRISPR)-CRISPR associated (Cas) (CRISPR-Cas) system comprising at least one Cas protein and a pair of guide RNAs (gRNAs), an engineered transposon system, at least one donor nucleic acid, and a recombinase. The present disclosure also provides methods for genetically modifying diverse bacterial communities comprising contacting a recipient bacterial community with donor bacteria, the donor bacteria comprising a vector encoding: an engineered CRISPR-Cas system, wherein the engineered CRISPR-Cas system comprises: at least one Cas protein and at least one guide RNA (gRNA); an engineered transposon system; at least one donor nucleic acid to be integrated comprising at least one transposon end sequence, and, optionally a recombinase, wherein the donor nucleic acid further comprises a recognitions site for the recombinase.

### CROSS-REFERENCE TO RELATED APPLICATIONS

This application claims the benefit of U.S. Provisional Application No. 63/001,008, filed Mar. 27, 2020, U.S. Provisional Application No. 63/053,460, filed Jul. 17, 2020, U.S. Provisional Application No. 63/081,677, filed Sep. 22, 2020, the content of each of the aforementioned applications is herein incorporated by reference in their entirety.

### FIELD

The present disclosure provides systems, kits, compositions, and methods for nucleic acid modification (e.g., deletion).

### BACKGROUND

The genetic engineering toolbox for genome manipulation comprises a diverse array of techniques, with DNA insertion technologies having arguably had the largest impact on biotechnology research. Gene knock-ins are used in the clinic to treat genetic diseases and cancer, in agriculture to improve crops, and in industry to manufacture biologics, among many other uses. These applications generally depend on either site-specific integration mediated by homologous recombination and gene editing, or random integration mediated by viral integrases or transposases. The former category is inherently precise but reliant on often-inefficient cellular factors or exogenous factors with limited host range, whereas the latter category exhibits high efficiency but little specificity. For certain genome engineering challenges, the ideal technology would exhibit high-efficiency DNA integration that bypasses the requirement for DNA double-strand breaks (DSBs) and homologous recombination, but with the specificity and programmability afforded by CRISPR-Cas gene-editing platforms.

### SUMMARY

00041 Provided herein are systems, kits, and methods that facilitate targeted nucleic acid deletions. The system comprise an engineered Clustered Regularly Interspaced Short Palindromic Repeats (CRISPR)-CRISPR associated (Cas) (CRISPR-Cas) system, and/or one or more vectors encoding the engineered CRISPR-Cas system, wherein the engineered CRISPR-Cas system comprises: (a) at least one Cas protein, and (b) a pair of guide RNAs (gRNAs), wherein the pair of gRNAs is configured to hybridize to target sites flanking a nucleic acid sequence for deletion; an engineered transposon system, and/or one or more vectors encoding the engineered transposon system; a recombinase, or catalytic domain thereof, and/or one or more vectors encoding a recombinase, or catalytic domain thereof; and at least one donor nucleic acid to be integrated, wherein the donor nucleic acid comprises a recognition site for the recombinase flanked by at least one transposon end sequence. In some embodiments, the donor nucleic acid further comprises a cargo nucleic acid. In some embodiments, the system further comprises a target nucleic acid comprising the nucleic acid sequence for deletion.

In some embodiments, the engineered CRISPR-Cas system and the engineered transposon system are on the same or different vector(s). In some embodiments, the recombinase, or catalytic domain thereof, is on the same or different vector(s) from the engineered CRISPR-Cas system and/or the engineered transposon system.

In some embodiments, the recombinase, or catalytic domain thereof, comprises a tyrosine recombinase. In some embodiments, the recombinase comprises Cre recombinase, a mutant, variant, or catalytic domain thereof and the recognition site comprises a lox site or variant thereof. In some embodiments, the recombinase comprises flippase (FLP) recombinase, a mutant, variant, or catalytic domain thereof and the recognition site comprises a flippase recognition target (FRT) site or variant thereof. In some embodiments, the recombinase, or catalytic domain thereof, comprises a serine recombinase. In some embodiments, the recombinase comprises TniR resolvase, a mutant, variant, or catalytic domain thereof and the recognition site comprises a resolution (res) sequence or variant thereof. In some embodiments, the recombinase comprises a Tn3-like resolvase, mutant, variant, or catalytic domain thereof and the recognition site comprises a resolution (res) sequence or variant thereof. In some embodiments, the cargo nucleic acid comprises the recognition site for the recombinase.

In some embodiments, the engineered CRISPR-Cas system comprises a Type V system or a Type I system. In some embodiments, the engineered CRISPR-Cas system comprises Cas12k. In some embodiments, the engineered CRISPR-Cas system comprises Cas5, Cas6, Cas7, Cas8, or a combination thereof. In some embodiments, the engineered CRISPR-Cas system comprises a Cas8-Cas5 fusion protein.

In some embodiments, the engineered transposon system is derived from a Tn7 transposon system. In some embodiments, the engineered transposon system comprises TnsA, TnsB, TnsC, or a combination thereof. In some embodiments, the engineered transposon system comprises TniQ.

Also provided herein is a cell comprising the present system. In some embodiments, the cell is a eukaryotic cell. In some embodiments, the nucleic acid sequence for deletion is an endogenous nucleic acid. In some embodiments, the nucleic acid sequence for deletion is genomic DNA. In some embodiments, the system is a cell-free system.

In some embodiments, the methods for deleting a nucleic acid sequence from a target nucleic acid comprise contacting the target nucleic acid with the present system. In some embodiments, the target nucleic acid is in a cell and contacting the target nucleic acid comprises introducing into the cell. In some embodiments, the recombinase, or catalytic domain thereof, and/or one or more vectors encoding a recombinase, or catalytic domain thereof is introduced to the cell after the introduction of the engineered CRISPR-Cas system, the engineered transposon system, and the at least one donor nucleic acid. In some embodiments, the recombinase, or catalytic domain thereof, and/or one or more vectors encoding a recombinase, or catalytic domain thereof is introduced to the cell after the introduction of the engineered CRISPR-Cas system, the engineered transposon system, and the at least one donor nucleic acid. In some embodiments, introducing into the cell comprises administering to a subject. In some embodiments, the administering comprises intravenous administration.

Also provide are methods for inactivating a gene of interest. The methods comprise introducing into one or more cells the present system, wherein the nucleic acid sequence for deletion comprises at least a portion of the gene of interest. In some embodiments, the one or more cells comprises microbial cells. In some embodiments, the one or more cells comprises plant cells. In some embodiments, the one or more cells comprises animal cells. In some embodiments, the gene of interest comprises an antibiotic resistance gene, a virulence gene, or a metabolic gene.

Further provided herein are methods for genetically modifying diverse bacterial communities. The methods comprise contacting a recipient bacterial community with donor bacteria, the donor bacteria comprising a vector encoding: an engineered Clustered Regularly Interspaced Short Palindromic Repeats (CRISPR)-CRISPR associated (Cas) (CRISPR-Cas) system, wherein the engineered CRISPR-Cas system comprises: (a) at least one Cas protein, and (b) at least one guide RNA (gRNA); an engineered transposon system; and at least one donor nucleic acid to be integrated comprising at least one transposon end sequence. In some embodiments, the donor nucleic acid further comprises a cargo nucleic acid. In some embodiments, the vector is a conjugative plasmid.

In some embodiments, the engineered CRISPR-Cas system comprises a Type V system or a Type I system. In some embodiments, the engineered CRISPR-Cas system comprises Cas12k. In some embodiments, the engineered CRISPR-Cas system comprises Cas5, Cas6, Cas7, Cas8, or a combination thereof. In some embodiments, the engineered CRISPR-Cas system comprises a Cas8-Cas5 fusion protein.

In some embodiments, the engineered transposon system is derived from a Tn7 transposon system. In some embodiments, the engineered transposon system comprises TnsA, TnsB, TnsC, or a combination thereof. In some embodiments, the engineered transposon system comprises TniQ.

In some embodiments, the vector further encodes a recombinase, or a catalytic domain thereof, and the at least one donor nucleic acid further comprises a recognition site for the recombinase. In some embodiments, the cargo nucleic acid comprises the recognition site for the recombinase.

In some embodiments, the recombinase, or catalytic domain thereof, comprises a tyrosine recombinase. In some embodiments, the recombinase comprises Cre recombinase, a mutant, variant, or catalytic domain thereof and the recognition site comprises a lox site or variant thereof. In some embodiments, the recombinase comprises flippase (FLP) recombinase, a mutant, variant, or catalytic domain thereof and the recognition site comprises a flippase recognition target (FRT) site or variant thereof. In some embodiments, the recombinase, or catalytic domain thereof, comprises a serine recombinase. In some embodiments, the recombinase comprises TniR resolvase, a mutant, variant, or catalytic domain thereof and the recognition site comprises a resolution (res) sequence or variant thereof. In some embodiments, the recombinase comprises a Tn3-like resolvase, mutant, variant, or catalytic domain thereof and the recognition site comprises a resolution (res) sequence or variant thereof. In some embodiments, the cargo nucleic acid comprises the recognition site for the recombinase. In some embodiments, the engineered CRISPR-Cas system comprises a pair of guide RNAs (gRNAs), wherein the pair of gRNAs is configured to hybridize to target sites flanking a nucleic acid sequence for deletion.

In some embodiments, the engineered CRISPR-Cas system, the engineered transposon system, the recombinase, or a combination thereof are encoded within the at least one donor nucleic acid. In some embodiments, the engineered CRISPR-Cas system, the engineered transposon system, the recombinase, or a combination thereof are encoded within the cargo nucleic acid.

In some embodiments, the nucleic acid sequence for deletion comprises a genomic nucleic acid sequence endogenous to the recipient bacterial community. In some embodiments, the recipient bacterial community is isolated from fecal matter. In some embodiments, the recipient bacterial community comprises gut bacteria.

### BRIEF DESCRIPTION OF THE DRAWINGS

FIGS. 1A-1F show streamlined single-plasmid system for RNA-guided DNA integration. FIG. 1A is a schematic of INTEGRATE (insertion of transposable elements by guide RNA-assisted targeting) using a Vibrio cholerae CRISPR-transposon. RNA-guided DNA integration occurs ˜47-51 bp downstream of the target site, in one of two possible orientations (T-RL and T-LR); the donor DNA comprises a genetic cargo flanked by left (L) and right (R) transposon ends (FIG. 1B). FIG. 1C, top, shows a three-plasmid INTEGRATE system which encodes protein-RNA components on pQCascade and pTnsABC, and the donor DNA on pDonor. On the bottom, a single-plasmid INTEGRATE system (pSPIN) drives protein-RNA expression with a single promoter, on the same vector as the donor DNA. FIG. 1D is a graph of qPCR-based quantification of integration efficiency with crRNA-4, for pSPIN containing distinct vector backbones of differing copy numbers. FIG. 1E is a graph of relative integration efficiencies for the three-plasmid or single-plasmid (pSPIN) expression system across five distinct crRNAs. Data are normalized to the three-plasmid system; pSPIN contained the pBBR1 backbone. FIG. 1F is normalized Tn-seq data for crRNA-13 and a non-targeting crRNA (crRNA-NT) for pSPIN containing the pBBR1 backbone. Genome-mapping reads are normalized to the reads from a spike-in control; the target site is denoted by a maroon triangle. Data in FIGS. 1D and 1E are shown as mean±s.d. for n=3 biologically independent samples.

FIGS. 2A-2E show INTEGRATE supports high-efficiency insertion of large (10-kb) genetic payloads. FIG. 2A is a graph of qPCR-based quantification of integration efficiency with crRNA-4 as a function of pSPIN promoter identity. FIG. 2B is a graph of DNA integration specificity (black) for the promoters shown, as determined by Tn-seq, calculated as the percent of on-target reads relative to all genome-mapping reads; total integration efficiencies (qPCR) are plotted in grey. FIG. 2C is a graph of qPCR-based quantification of integration efficiency for crRNA-4 as a function of culture temperature and promoter strength. Integration reaches ˜100% efficiency at lower growth temperatures for all constructs, including the weaker J23114 promoter. FIG. 2D is graphs of qPCR-based quantification of integration efficiency for variable mini-Tn sizes after culturing at either 30 or 37° C. The promoter and crRNA used in each panel are shown at top; experiments were performed with a two-plasmid system comprising pEffector (pEffector-B, FIG. 6C) and pDonor. Unless specified, transposition assays elsewhere in this study use a 0.98-kb mini-Tn. FIG. 2E shows qPCR-based quantification of integration efficiency with crRNA-4 for a single-plasmid autonomous INTEGRATE system (pSPAIN), after culturing at 30 and 37° C. The inserted DNA encodes all the necessary machinery for further mobilization. Integration efficiency data in FIGS. 32A-3E are shown as mean±s.d. for n=3 biologically independent samples.

FIGS. 3A-3D show orthogonal INTEGRATE systems facilitate multiple, iterative insertions. FIG. 3A shows the effect of target immunity on RNA-guided DNA integration. An E. coli strain containing a single genomically integrated mini-Tn was generated, and the efficiency of additional transposition events using crRNAs targeting d bp upstream was determined by qPCR. Plotted is the relative efficiency for each crRNA in the immunized versus wild-type strain. FIG. 3B, top, is a schematic showing re-mobilization of a genomically integrated mini-Tn (target-4) to a new genomic site (target-1) with crRNA-1. FIG. 3B, bottom, shows PCR products probing for the mini-Tn at target-4 (left) and target−1 (right), resolved by agarose gel electrophoresis. The mini-Tn is efficiently transposed to target-1 by crRNA-1, without apparent loss of the mini-Tn at target-4. FIG. 3C, top, is a schematic of orthogonal INTEGRATE systems from V. cholerae (Vch: Type I-F) and S. hofmannii (Sho; Type V-K), in which pDonor is separate from pEffector. FIG. 3C, bottom, shows PCR products probing for RNA-guided DNA integration at target-4 with both systems, resolved by gel electrophoresis. Integration only proceeds with a cognate pairing between the expression and donor plasmids. FIG. 3D, top, is a schematic of a second DNA insertion made by leveraging the orthogonal ShoINT system, for which the Vch mini-Tn is inert. FIG. 3D, bottom, shows PCR products probing for either the Vch mini-Tn (top) or Sho mini-Tn (bottom) at target-4 (left) and target−1 (right), resolved by agarose gel electrophoresis. The Sho mini-Tn is efficiently integrated at target-1 by sgRNA-1, without loss of the Vch mini-Tn at target-4. Data in a are shown as mean±s.d. for n=3 biologically independent samples.

FIGS. 4A-4I show multi-spacer CRISPR arrays direct multiplex insertions in a single step. FIG. 4A is a schematic of multiplexed RNA-guided DNA integration events with pSPIN encoding a multi-spacer CRISPR array. FIG. 4B is a graph of qPCR-based quantification of integration efficiency with crRNA-NT (grey) and crRNA-4 (maroon), encoded in a single-, double-, or triple-spacer CRISPR array in the position indicated: white squares represent other functional crRNAs. Data are normalized to the single-spacer array efficiency. FIG. 4C shows Tn-seq data for a triple-spacer CRISPR array, plotted as the percent of total genome-mapping reads. The target sites are denoted by colored triangles, and the insets show the distribution of integration events within a 42-58 bp window downstream of the target site. FIG. 4D is a schematic of experiment using thrC- and lysA-specific spacers for single-step generation of threonine-lysine auxotrophic E. coli. FIG. 4E is a graph of the recovery percentage of the indicated clonal genotypes (WT, single-knockout, or double-knockout) after transforming E. coli with pSPIN encoding a double-spacer CRISPR array containing both hrC- and lynA-specific spacers. Colonies were stamped onto M9-agar supplemented with either H₂O, threonine, lysine, or threonine and lysine, and genotypes were determined based on survivability in each of these conditions. FIG. 4F is growth curves for WT and double-knockout E. coli clones cultured at 37° C. in LB or M9 minimal media with or without supplemented threonine (T) and lysine (L). FIG. 5G is a schematic of experimental approach to generate programmed genomic deletions. A double-spacer array directs multiplex insertion of two mini-Tn copies carrying LoxP sites: subsequent introduction of Crc recombinase leads to precise excision of the genomic fragment spanning the LoxP sites. FIG. 4H, left, is a schematic showing genomic locus targeted for deletion. FIG. 4H, right, shows the indicated double-spacer arrays were used to generate defined deletions, and PCR was performed with the indicated primer pairs to detect the presence/absence of genomic fragments flanking each target site, resolved by agarose gel electrophoresis. FIG. 4I shows that the programmed genomic deletions generated in FIG. 4H (2.4-, 10-, or 20-kb in length) were further verified by whole-genome, single-molecule real-time (SMRT) sequencing. The mini-transposon is indicated with an arrow. Data in FIG. 4B and FIG. 4E are shown as mean±s.d. for n=3 biologically independent samples. Data in f are shown as mean±s.d. for three technical replicates.

FIGS. 5A-5E show robust and highly-accurate INTEGRATE activity in additional Gram-negative bacteria. FIG. 5A is a schematic showing the use of pSPIN constructs with constitutive J23119 promoter and broad-host pBBR1 backbone for RNA-guided DNA insertions in Klebsiella oxytoca and Pseudomonas putida with corresponding micrographs. FIG. 5B shows PCR products probing for mini-Tn insertion at two different genomic loci in K. oxytoca (left) and P. putida (right), resolved by agarose gel electrophoresis. FIG. 5C is normalized Tn-seq data for select targeting and non-targeting crRNAs for K. oxytoca (top) and P. putida (bottom). Genome-mapping reads are normalized to the reads from a spike-in control: the target site is denoted by a maroon triangle. FIG. 5D, top, is a schematic showing self-targeting of the spacer within the CRISPR array inactivates the pSPIN-encoded INTEGRATE system, and was detected for select crRNAs by Tn-seq (FIG. 5D, middle). FIG. 5D, bottom, is a graph showing P. putida crRNAs targeting nicC and bdhA, but not nirD, show substantial plasmid self-targeting relative to genomic integration, as assessed by Tn-seq. FIG. 5D, top, is a schematic showing a modified vector (pSPIN-R) places the CRISPR array proximal to the mini-Tn, whereby self-targeting is blocked by target immunity. FIG. 5D, bottom, is a graph showing P. putida crRNAs targeting nicC and bcAA no longer show any evidence of self-targeting with pSPIN-R, as assessed by Tn-seq.

FIGS. 6A-6C show the reduction of promoter and plasmid requirements for RNA-guided DNA integration. FIG. 6A is a schematic illustrating Cas6-dependent processing of an RNA transcript comprising precursor CRISPR RNA and polycistronic mRNA, which liberates the mature crRNA; CRISPR repeats are shown as hairpins. FIG. 6B shows three pQCascade designs containing either two or one T7 promoters, with the CRISPR array either upstream of downstream of the operon, top, and qPCR-based quantification of integration efficiency with crRNA-4 (bottom). Cells contained pDonor, pTnsABC, and the indicated pQCascade construct. FIG. 6C shows four protein-RNA expression plasmid constructs containing either two or one T7 promoters, with the CRISPR array either upstream of downstream of the operon (top) and qPCR-based quantification of integration efficiency with crRNA-4 (bottom). Cells contained pDonor and the indicated expression plasmid. Data in FIGS. 6B and 6C are shown as mean±s.d. for n=3 biologically independent samples.

FIGS. 7A-7C show Mini-Tn vector context effects on integration orientation. FIG. 7A is graphs of integration efficiencies in the T-RL and T-LR orientation are plotted from experiments in FIG. 1E, for the three-plasmid and single-plasmid expression systems. Integration is more heavily biased towards T-RL for the single-plasmid system, particularly for crRNA-4. FIG. 7B is a schematic of the original pDonor plasmid, which contains a lac promoter upstream of the transposon right end, and a modified pDonor plasmid in which this promoter was removed. The modified pDonor shows more frequent T-RL integration, which may be due to the absence of active transcription across the right (R) transposon end. FIG. 7C is a comparison of integration orientation bias (T-RL:T-LR) for the three-plasmid expression system with crRNA-4, using the original or modified pDonor; efficiencies were measured by qPCR. Data in FIGS. 7A and 7C are shown as mean±s.d. for n=3 biologically independent samples.

FIGS. 8A-8E show Genome-wide analysis of RNA-guided DNA integration by Tn-seq. FIG. 8A is an exemplary Tn-seq workflow for deep sequencing of genome-wide transposition events. FIG. 8B shows genome-wide distribution of genome-mapping Tn-seq reads for crRNA-1 and crRNA-4 using either the single-plasmid or three-plasmid expression system; the target site is denoted by a maroon triangle. FIG. 8C is Tn-seq for additional crRNAs using the single-plasmid expression system, shown as in FIG. 8B. FIG. 8D is integration site distributions for crRNA-1 (top) and crRNA-4 (bottom) using either the single-plasmid or three-plasmid expression system, determined from the Tn-seq data; the distance between the target site and mini-Tn insertion site is shown. Data for both integration orientations are superimposed, with filled blue bars and dark outlines representing T-RL and T-LR, respectively. Values in the top-right corner of each graph give the on-target specificity (%), calculated as the percentage of reads resulting from integration within 100 bp of the primary integration site compared to all genome-mapping reads, and the orientation bias (X:Y), calculated as the ratio of T-RL:T-LR reads within the on-target window. FIG. 8E is integration site distributions for additional crRNAs using the single-plasmid expression system, shown as in FIG. 8D.

FIGS. 9A-9G show Analysis of genome-wide integration specificity as a function of promoter strength, cargo size, and E. coli strain. FIG. 9A is integration site distributions for crRNA-4 as a function of promoter strength, determined from the Tn-seq data; the distance between the target site and mini-Tn insertion site is shown. Data for both integration orientations are superimposed, with filled blue bars and dark outlines representing T-RL and T-LR, respectively. Values in the top-right corner of each graph give the on-target specificity (%), calculated as the percentage of reads resulting from integration within 100 bp of the primary integration site compared to all genome-mapping reads, and the orientation bias (X:Y), calculated as the ratio of T-RL:T-LR reads within the on-target window. FIG. 9B is integration site distributions for crRNA-13, determined for three different laboratory strains of E. coli, shown as FIG. 9A. FIG. 9C is qPCR-based quantification of integration efficiency for crRNA-13 in the indicated Keio knockout strains; integration efficiency was reduced for the ΔrecB and ΔrecC strains, but unaffected in ΔrecA, ΔrecD, ΔrecF, and ΔmutS strains. Data are normalized to the efficiency in the WT BW25113 parental strain. FIG. 9D is integration site distribution for crRNA-4 under control of the J23119 promoter after cells were cultured at 30° C., shown as in FIG. 9A. FIG. 9E is qPCR-based quantification of integration efficiency for variable mini-Tn sizes after culturing at either 30 or 37° C. The promoter and crRNA used are shown at top. FIG. 9F is integration site distributions for crRNA-4 as a function of cargo size, shown as in. FIG. 9G is whole-genome, single-molecule real-time (SMRT) sequencing data for an isolated clone containing the 10-kb insertion, shown as coverage of aligned reads across the entire locus. Data in FIGS. 9C and 9E are shown as mean±s.d. for n=3 biologically independent samples.

FIGS. 10A-10E show Evaluation of mini-Tn remobilization by Vch INTEGRATE, and characterization of anew Type V-K S. hofmannii INTEGRATE system. FIG. 10A shows a schematic (left) showing potential competition between a genomic- and pDonor-borne mini-Tn when a new site is targeted for RNA-guided DNA integration; the two possible products can be discriminated by cargo-specific primer binding sites. PCR products probing for transposition of the genomic mini-Tn (FIG. 10A, right, top) or pDonor-borne mini-Tn (FIG. 10A, right, bottom) to the target-1 locus. Although pDonor is the preferred substrate, there is also detectable re-mobilization of the genomic mini-Tn substrate, without apparent loss of the mini-Tn at target-4. FIG. 10B is a schematic of native genomic organization of a Type VK CRISPR-transposon encoding Cas12k, found within the genome of Scytonema hofmannii (Sho) strain PCC 7110 (top), and plasmid constructs used to recombinantly express the sgRNA and protein components (Sho-pGCT) and the mini-Tn (Sho-pDonor) (bottom). FIG. 10C shows the genomic locus targeted by sgRNAs 31-34 (top), and PCR analysis of transposition by ShoINT, resolved by agarose gel electrophoresis (bottom). Bidirectional integration was observed in both T-RL and T-LR orientations for multiple sgRNAs, though there is a strong bias for T-LR. FIG. 10D is an overview of RNA-guided DNA integration by ShoINT. Insertion occurs in two possible orientations, similarly to the Type I-F VchINT system, at an approximate distance of 25-35 bp from the edge of the target site. The 4-nt PAM and 23-nt protospacer are shown as orange and maroon rectangles, respectively. FIG. 10E is a graph of qPCR-based quantification of integration efficiency for sgRNAs. Data in FIG. 10E are shown as mean±s.d. for n=3 biologically independent samples.

FIGS. 11A-11C show Analysis of genome-wide integration events for three CRISPR-transposon systems. FIG. 11A is a comparison of two distinct next-generation sequencing (NGS) library preparation techniques for analyses of genome-wide integration specificity with VchINT: transposon-insertion sequencing (Tn-seq), based on restriction digestion and adaptor ligation onto mini-Tn-containing genomic fragments, followed by targeted PCR; and random fragmentation and adaptor ligation onto all genomic fragments, followed by targeted PCR. The target site is denoted by a maroon triangle. Insets show integration site distributions determined from the NGS data; the distance between the target site and mini-Tn insertion site is shown. Data for both integration orientations are superimposed, with filled blue bars and dark outlines representing T-RL and T-LR, respectively. Values in the top-right corner of each graph give the on-target specificity (%), calculated as the percentage of reads resulting from integration within 100 bp of the primary integration site compared to all genome-mapping reads, and the orientation bias (X:Y), calculated as the ratio of T-RL:T-LR reads within the on-target window. Both analyses return highly consistent data. FIG. 11B is the analysis of genome-wide integration specificity with ShoINT and the ShCAST system described previously, shown as in FIG. 11A. ShoINT exhibited high levels of integration into the T7 RNAP gene (†), suggesting a cellular fitness benefit when expression of the recombinant protein-RNA machinery is eliminated through T7 RNAP inactivation. FIG. 11C is a comparison of genome-wide specificity between VchINT (Type I-F), ShoINT (Type V-K), and ShCAST (Type V-K) as assessed via random fragmentation-based NGS library preparation, shown as in a but focused on reads comprising 1% or less of the library. The Type IF system exhibits exquisite accuracy, whereas both Type V-K systems exhibit rampant nonspecific integration across the E. coli genome. *, low-level, well-to-well contamination of NGS data from other samples.

FIGS. 12A and 12B show genome-wide analysis of multiplexed RNA-guided DNA integration. FIG. 12A is genome-wide distribution of genome-mapping Tn-seq reads for a double-spacer (top) and triple-spacer (bottom) CRISPR array; the corresponding target sites are denoted by similarly colored triangles. The top graphs plot the percentage of total reads; the bottom graphs focus on reads comprising 1% or less of the library, revealing an absence of detectable off-target events. The overall on-target percentages combine all reads mapping to the on-target window of each individual genomic target. FIG. 12B shows integration site distributions for the indicated crRNA as a function of CRISPR array composition, determined from the Tn-seq data: the distance between the target site and mini-Tn insertion site is shown. Data for both integration orientations are superimposed, with filled blue bars and dark outlines representing T-RL and T-LR, respectively.

FIG. 13A-13D show generation of auxotrophic E. coli strains through single- or multiplex integration. FIG. 13A is a workflow for generating and screening auxotrophic E. coli knockouts with multiplexed RNA-guided DNA integration. FIG. 13B is growth curves for single-knockout E. coli clones cultured at 37° C. in LB or M9 minimal media with or without supplemented threonine (T) and lysine (L). FIG. 13C is growth curves for WT or control E. coli clones transformed with a non-targeting crRNA (crRNA-NT), cultured at 37° C. in LB or M9 minimal media with or without supplemented threonine (T) and lysine (L). FIG. 13D is growth curves for double-knockout E. coli clone cultured at 37° C. in LB or M9 minimal media with or without supplemented threonine (T) and lysine (L), after five cycles of serial passaging and overnight growth in LB media. Data in FIGS. 13B-13D are shown as mean f s.d. for three technical replicates.

FIGS. 14A-14C show SMRT sequencing of programmed deletions using INTEGRATE and Cre-Lox. FIG. 14A shows a schematic (top) of genomic locus targeted for a 2.4-kb deletion with the double-spacer CRISPR array shown at the right; triangles represent corresponding target sites and coverage data from whole-genome SMRT sequencing reads from an isolated clone, aligned to the E. coli BL21(DE3) reference genome (bottom). FIG. 14B is 10-kb deletion data, shown as in FIG. 14A. FIG. 14C is 20-kb deletion data, shown as in FIG. 14A.

FIGS. 15A-15F shows Genome-wide analysis of RNA-guided DNA integration in K. oxytoca and P. putida. FIG. 15A is genome-wide distribution of genome-mapping Tn-seq reads for the indicated crRNA expressed by pSPIN-BBR1 in K. oxytoca; the target site is denoted by a maroon triangle. FIG. 15B is genome-wide distribution of genome-mapping Tn-seq reads for the indicated crRNA expressed by pSPIN-BBR1 in P. putida; the target site is denoted by a maroon triangle. ‡, off-target integration site. FIG. 15C is integration site distributions for the indicated crRNAs in K. oxytoca, determined from the Tn-seq data; the distance between the target site and mini-Tn insertion site is shown. Data for both integration orientations are superimposed, with filled blue bars and dark outlines representing T-RL and T-LR, respectively. Values in the top-right corner of each graph give the on-target specificity (%), calculated as the percentage of reads resulting from integration within 100 bp of the primary integration site compared to all genome-mapping reads, and the orientation bias (X:Y), calculated as the ratio of T-RL:T-LR reads within the on-target window. FIG. 15D is integration site distributions for the indicated crRNAs in P. putida, shown as in FIG. 15C. FIG. 15E is integration site distributions for the off-target peak (‡) with crRNA-51 in P. putida, shown in FIG. 15C. The sequences of the on-target and off-target sequences upstream of the integration site are shown to the right, highlighting the high degree of sequence similarity. FIG. 15F is integration site distributions for the indicated crRNAs in P. putida, shown as in FIG. 15D; these experiments utilized the reversed pSPIN-R plasmid, as compared to the pSPIN plasmid used in FIG. 15D.

FIG. 16 is a Flowchart for the INTEGRATE guide RNA design algorithm. Spacers with a defined length and PAM are generated and filtered from a given reference genome, based on the target gene name or genomic coordinates. The Bowtie2 alignment tool60 is used to evaluate each spacer candidate for potential off-targets genome-wide. Spacers are considered to have potential off-targets when Bowtie2 detects alignments exhibiting less than a user-specified maximum mismatch limit. For bacterial genomes, this process usually results in a sufficient number of spacers within each window, without the need for scoring each spacer candidate. For Type I Cascade (such as VchINT) spacers, the program converts flexible bases—those bases occurring every 6th position, which do not contribute to spacer-protospacer complementarity within the R-loop—to ‘N’ to exclude these bases from contributing to the mismatch count for the genome-wide off-target search. The off-target search module can also be executed separately for the evaluation of user-specified spacers. The program and more in-depth documentation are publicly accessible via GitHub (github.com/sternberglab/INTEGRATEguide-RNA-tool).

FIGS. 17A-17F show in vivo kinetics of RNA-guided transposition. FIG. 17A are graphs of integration over a 24-h time course at either 30 or 37° C., using pSPIN encoding crRNA-4 driven by either a strong (J23119, left) or weak (J23114, right) promoter. At each time point, integration efficiencies and culture growth states were determined by qPCR (top) and OD600 (bottom) measurements, respectively. FIG. 17B is a graph of integration for the 37° C. culture from FIG. 17A (J23119 promoter) was diluted 1:200 into fresh LB media at the indicated timepoint. Integration efficiencies and culture growth states were determined as in FIG. 17A. FIG. 17C is PCR analysis of T-RL integration for samples collected from the 37° C. cultures in FIG. 17A. Integration can be detected within 2 hours after transformation. FIG. 17D is a schematic of a transposition experiment where integration was performed using pEffector-B and a transposon donor delivered as a purified linear PCR amplicon. The mini-Tn encodes a chloramphenicol resistance cassette. FIG. 17E is PCR analysis of T-RL integration at target-4 from transposition assays using a linear PCR amplicon mini-Tn. Integration was readily detected in 6/6 colonies selected for chloramphenicol resistance. FIG. 17F is a graph of quantification of colony forming units (CFU) on LB chloramphenicol plates from transposition experiments using linear PCR amplicon mini-Tn and pEffector-B encoding either crRNA-4 or a non-targeting (NT) crRNA. Data in FIGS. 17A and 17B are shown as mean±s.d. for n=3 biologically independent samples.

FIGS. 18A-18F show Programmable integration within a complex bacterial community. FIG. 18A is a schematic of an exemplary experiment, in which pSPIN is delivered by conjugation from a donor E. coli strain into a complex bacterial community derived from the mouse gut. pSPIN was designed to specifically target the IacZ locus of K. oxytoca strain M5a1, which was added to the community before conjugation. FIG. 18B is 16S sequencing indicating that the gut microbiome communities 1 and 2 (C1 and C2, extracted from B6 and BALB/C mice, respectively) had diverse taxa. The barplots represent the relative abundance of different phyla in the commensal communities when donor and recipients were first introduced (Time 0 h) and 24 h after anaerobic growth in MGAM (Time 24 h). Data represent the average of three replicates. FIG. 18C is the PCR analysis of T-RL integration into the K. oxytoca lacZ target site from a population of recipient cells. Integration occurs robustly across both communities with the targeting crRNA (crRNA-41) but not a non-targeting (NT) crRNA. PCR products are shown for three biological replicates of conjugation experiments with communities 1 and 2, and for two distinct donor-to-recipient ratios tested. FIG. 18D is the Sanger sequencing of a representative PCR product from FIG. 18C confirming site-specific integration into the target K. oxytoca lacZ locus. The imperfect alignment observed at the genome-transposon junction is characteristic of variable integration sites across the population 35. FIG. 18E is representative T-RL PCR products assayed from isolated K. oxytoca colonies after the conjugation experiments into community 2. Integration is detected in 10/10 colonies. Colonies were obtained from LB agar plates with selection for pSPIN (but not for the integration event), and were confirmed to be K. oxytoca by independent 16S Sanger sequencing. FIG. 18F is a graph of the quantification of K. oxytoca colonies that underwent targeted integration by PCR analysis of T-RL. 40-66 colonies were analyzed for each conjugation condition, and colonies were confirmed to be K. oxytoca by independent 16S Sanger sequencing. Data in FIG. 18F are shown as mean±s.d. for n=3 biologically independent samples.

FIG. 19 is a chart of the protein sequence similarity between different transposase systems. Analysis was performed using BLASTp with default options. TnsA was not analyzed as both shCAST and ShoINT systems lack TnsA.

FIG. 20 is a chart of bacterial species and strains.

FIG. 21 is a zoomed-in view for the bile salt hydrolase (BSH) gene from Bacteroides vulgatus showing the three spacer-matching sites. The three sites targeted by Bacteroides Spacer1, Spacer2, and Spacer3 are shown, within the context of the BSH gene.

FIG. 22 is an image of the junction PCR analysis of targeted integration products from Bacteroides vulgatus transconjugants after delivery of pSPIN encoding a guide RNA with Spacer1. Primers were designed to amplify either tRL or tLR products after targeted integration programmed by a guide RNA with Spacer1, in which the transposon is integrated with either the ‘right’ or ‘left’ end proximal to the target site, respectively. Primer pairs comprise a genome-specific primer and a donor DNA-specific primer. The expected band sizes for on-target insertion products are ˜0.75-1.0 kb for the two orientations. Each lane underneath the “RL” and “LR” regions represents a singly transconjugant colony subjected to junction PCR analysis. Representative bands were subsequently excised and analyzed by Sanger sequencing.

FIG. 23 is an image of the junction PCR analysis of targeted integration products from Bacteroides vulgatus transconjugants after delivery of pSPIN encoding a guide RNA with Spacer2. Primers were designed to amplify either tRL or tLR products after targeted integration programmed by a guide RNA with Spacer2, in which the transposon is integrated with either the ‘right’ or ‘left’ end proximal to the target site, respectively. Primer pairs comprise a genome-specific primer and a donor DNA-specific primer. The expected band sizes for on-target insertion products are ˜0.75-1.0 kb for the two orientations. Each lane underneath the “RL” and “LR” regions represents a singly transconjugant colony subjected to junction PCR analysis. Representative bands were subsequently excised and analyzed by Sanger sequencing. This spacer has a clear bias for tRL integration products.

FIG. 24 is an image of the junction PCR analysis of targeted integration products from Bacteroides vulgatus transconjugants after delivery of pSPIN encoding a guide RNA with Spacer3. Primers were designed to amplify either tRL or tLR products after targeted integration programmed by a guide RNA with Spacer3, in which the transposon is integrated with either the ‘right’ or ‘left’ end proximal to the target site, respectively. Primer pairs comprise a genome-specific primer and a donor DNA-specific primer. The expected band sizes for on-target insertion products are ˜0.75-1.0 kb for the two orientations. Each lane underneath the “RL” and “LR” regions represents a singly transconjugant colony subjected to junction PCR analysis. Representative bands were subsequently excised and analyzed by Sanger sequencing. This spacer has a clear bias for tRL integration products.

FIG. 25 is the Sanger sequencing verification of on-target tRL product upon RNA-guided DNA integration in the Bacteroides vulgatus genome (SEQ ID NO: 261), after delivery of pSPIN encoding a guide RNA with Spacer1. Sanger sequencing chromatograms are shown using primers extending from either end of the junction PCR products, denoted here as “Forward read” and “Reverse read.” The chromatograms are aligned to a reference genome, showing the precise insertion of the transposon ‘left end’ (or left flank) 49-bp downstream of the genomic site complementary to the spacer. The target site duplication (TSD) is indicated.

FIG. 26 is the Sanger sequencing verification of on-target tRL product upon RNA-guided DNA integration in the Bacteroides vulgatus genome (SEQ ID NO: 262), after delivery of pSPIN encoding a guide RNA with Spacer2. Sanger sequencing chromatograms are shown using primers extending from either end of the junction PCR products, denoted here as “Forward read” and “Reverse read.” The chromatograms are aligned to a reference genome, showing the precise insertion of the transposon ‘left end’ (or left flank) 49-bp downstream of the genomic site complementary to the spacer. The target site duplication (TSD) is indicated.

FIG. 27 is the Sanger sequencing verification of on-target tRL product upon RNA-guided DNA integration in the Bacteroides vulgatus genome (SEQ ID NO: 263), after delivery of pSPIN encoding a guide RNA with Spacer3. Sanger sequencing chromatograms are shown using primers extending from either end of the junction PCR products, denoted here as “Forward read” and “Reverse read.” The chromatograms are aligned to a reference genome, showing the precise insertion of the transposon ‘left end’ (or left flank) 50-bp downstream of the genomic site complementary to the spacer. The target site duplication (TSD) is indicated.

### DETAILED DESCRIPTION

The disclosed systems, kits, compositions, and methods advance RNA-guided nucleic acid integration for efficient and multiplexed bacterial genome engineering.

DNA technologies to stably integrate genes and pathways into the genome enable the generation of engineered cells with entirely new functions. Applications of this powerful approach have already yielded impactful commercial products, with examples including CAR-T cell therapies, genetically modified crops, and cell factories producing diverse compounds and medicines. In many of these applications, genomic integration is highly preferred over plasmid-based methods for maintaining heterologous genes in engineered cells, due to improved stability in the genome, better control of copy numbers, and regulatory concerns regarding biocontainment of recombinant DNA. However, generation of modified cells with kilobases of changes across the genome remains practically challenging, often requiring inefficient, multi-step processes that are time and resource intensive.

In bacteria, genome engineering and integration can be achieved through several approaches that utilize endogenous or foreign integrases, transposases, recombinases, or homologous recombination (HR) machinery, which can be further combined with CRISPR-Cas to improve efficiency. While widely used, these methods are not without significant drawbacks. For example, recombination-mediated genetic engineering (recombineering) using X-red or RecET recombinase systems in E. coli allows programmable genomic integrations, specified by the homology arms flanking the foreign DNA cassette. However, recombineering efficiency is generally low (less than 1 in 103-104) without selection of a co-integrating selectable marker or CRISPR-Cas-mediated counter-selection of unedited alleles, and thus cannot be easily multiplexed to make simultaneous insertions into the same cell. There is a limited number of robust selectable markers (e.g., antibiotic resistance genes) that require another excision step to remove from the genome for subsequent reuse, and expression of Cas9 for negative selection can cause unintended DNA double-strand breaks (DSBs) that lead to cytotoxicity. Practically, recombineering has a payload size limit of only 3-4 kb in many cases, making it less useful for genomic integration of pathway-sized DNA cassettes. Finally, unknown requirements for host-specific factors or cross-species incompatibilities of phage recombination proteins have rendered E. coli recombineering systems more challenging to port to other bacteria, requiring significant species-specific optimizations or screening of new recombinases.

Other integrases and transposases, such as ICEBs and Tn7, have also been used for genome integration. These systems recognize highly specific attachment sites that are unfortunately difficult to reprogram, and thus require the prior presence of these sites or their separate introduction in the genome. Other more portable transposons, such as Mariner and Tn5, generate non-specific integrations that have been used for genome-wide transposon mutagenesis libraries. However, these transposases cannot be targeted to specific genomic loci, and large-scale screens are needed to isolate desired clones. More recently, a catalytically-dead Cas9 has been fused to either a transposase or a recombinase to provide better site specificity, which showed success in mostly in vitro studies. Autocatalytic Group II RNA introns, selfish genetic elements in bacteria, have also been used for genomic transpositions and insertions. This system utilizes an RNA intermediate to guide insertions, but suffers from inconsistent efficiencies ranging from 1-80% depending on the target site and species, and a limited cargo size of 1.8 kb.

A new category of programmable integrases was recently described in which sequence specificity is governed exclusively by guide RNAs. Motivated by the bioinformatic description of Tn7-like transposons encoding nuclease-deficient CRISPR-Cas systems, a candidate CRISPR-transposon from Vibrio cholerae (Tn6677) was selected and RNA-guided transposition was reconstituted in an E. coli host. DNA integration occurred ˜47-51 base pairs (bp) downstream of the genomic site targeted by the CRISPR RNA (crRNA), and required transposition proteins TnsA, TnsB, and TnsC, in conjunction with the RNA-guided DNA targeting complex TniQ-Cascade. Remarkably, bacterial transposons have hijacked at least three distinct CRISPR-Cas subtypes. The Type V-K effector protein. Cas12k, also directs targeted DNA integration, albeit with lower fidelity.

INTEGRATE (insertion of transposable elements by guide RNA-assisted targeting) benefits from both the high-efficiency, seamless integrations of transposases, as well as the simple programmability of CRISPR-mediated targeting. However, the system previously demonstrated in E. coli required multiple cumbersome genetic components and displayed low efficiency for larger insertions in dual orientations. Herein, an improved INTEGRATE system was developed that used streamlined expression vectors to direct highly accurate insertions at ˜100% efficiency, effectively in a single orientation, independent of the cargo size, without requiring selection markers.

Since INTEGRATE does not rely on homology arms specific to each target site, multiple simultaneous genomic insertions into the same cell could be rapidly generated using CRISPR arrays with multiple targeting spacers, and INTEGRATE paired with Cre-Lox was used to achieve genomic deletions. Using INTEGRATE is preferable for efficient and targetable genomic deletions in both prokaryotic and eukaryotic nucleic acids over previous methods due to the mechanism of action not utilizing double-strand breaks in the target nucleic acid, particularly in bacteria, and selective targeting to a nucleic acid sequence of interest for deletion. This allows a single construct to be employed in a plurality of bacteria or bacterial species for simultaneous deletions of the exact genomic region in each individual bacterium.

The portability and high site specificity of INTEGRATE was demonstrated in other species, including Klebsiella oxytoca, Pseudomonas putida, and Bacteroides vulgatus highlighting its broad utility for bacterial genome engineering. INTEGRATE was an effective genetic tool for engineering specific strands in a complex mammalian gut microbiome.

Section headings as used in this section and the entire disclosure herein are merely for organizational purposes and are not intended to be limiting.

### 1. DEFINITIONS

The terms “comprise(s),” “include(s),” “having.” “has,” “can,” “contain(s).” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific, and technical terms used in connection with the present disclosure shall have the meanings that are commonly understood by those of ordinary skill in the art. For example, any nomenclature used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

As used herein, a “nucleic acid” or a “nucleic acid sequence” refers to a polymer or oligomer of pyrimidine and/or purine bases, preferably cytosine, thymine, and uracil, and adenine and guanine, respectively (See Albert L. Lehninger, Principles of Biochemistry, at 793-800 (Worth Pub. 1982)). The present technology contemplates any deoxyribonucleotide, ribonucleotide, or peptide nucleic acid component, and any chemical variants thereof, such as methylated, hydroxymethylated, or glycosylated forms of these bases, and the like. The polymers or oligomers may be heterogenous or homogenous in composition and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholino nucleic acid (see, e.g., Braasch and Corey, Biochemistry, 41(14): 4503-4510 (2002)) and U.S. Pat. No. 5,034,506), locked nucleic acid (LNA; see Wahlestedt et al., Proc. Natl. Acad. Sci. U.S.A., 97: 5633-5638 (2000)), cyclohexenyl nucleic acids (see Wang, J. Am. Chem. Soc., 122: 8595-8602 (2000)), and/or a ribozyme. Hence, the term “nucleic acid” or “nucleic acid sequence” may also encompass a chain comprising non-natural nucleotides, modified nucleotides, and/or non-nucleotide building blocks that can exhibit the same function as natural nucleotides (e.g., “nucleotide analogs”); further, the term “nucleic acid sequence” as used herein refers to an oligonucleotide, nucleotide or polynucleotide, and fragments or portions thereof, and to DNA or RNA of genomic or synthetic origin, which may be single or double-stranded, and represent the sense or antisense strand. The terms “nucleic acid,” “polynucleotide,” “nucleotide sequence,” and “oligonucleotide” are used interchangeably. They refer to a polymeric form of nucleotides of any length, either deoxyribonucleotides or ribonucleotides, or analogs thereof.

Nucleic acid or amino acid sequence “identity.” as described herein, can be determined by comparing a nucleic acid or amino acid sequence of interest to a reference nucleic acid or amino acid sequence. The percent identity is the number of nucleotides or amino acid residues that are the same (e.g., that are identical) as between the sequence of interest and the reference sequence divided by the length of the longest sequence (e.g., the length of either the sequence of interest or the reference sequence, whichever is longer). A number of mathematical algorithms for obtaining the optimal alignment and calculating identity between two or more sequences are known and incorporated into a number of available software programs. Examples of such programs include CLUSTAL-W. T-Coffee, and ALIGN (for alignment of nucleic acid and amino acid sequences), BLAST programs (e.g., BLAST 2.1, BL2SEQ, and later versions thereof) and FASTA programs (e.g., FASTA3×, FAST™, and SSEARCH) (for sequence alignment and sequence similarity searches). Sequence alignment algorithms also are disclosed in, for example, Altschul et al., J. Molecular Biol., 215(3): 403-410 (1990), Beigert et al., Proc. Natl. Acad. Sci. USA, 106(10): 3770-3775 (2009), Durbin et al., eds., Biological Sequence Analysis: Probabilistic Models of Proteins and Nucleic Acids, Cambridge University Press, Cambridge, UK (2009), Soding, Bioinformatics, 21(7): 951-960 (2005), Altschul et al., Nucleic Acids Res., 25(17): 3389-3402 (1997), and Gusfield, Algorithms on Strings, Trees and Sequences, Cambridge University Press. Cambridge UK (1997)).

The terms “microbe” or microorganism” are used interchangeably herein to refer to prokaryotic and eukaryotic microbial species from the domains Archaea, Bacteria and Eucarya, the latter including yeast and filamentous fungi, protozoa, algae, and higher Protista. “Microbial cells” refer to cells derived from a microbe or microorganism, as defined herein, or, in the case of single-celled organisms, the organism itself.

A “vector” or “expression vector” is a replicon, such as plasmid, phage, virus, or cosmid, to which another DNA segment, e.g., an “insert,” may be attached or incorporated so as to bring about the replication of the attached segment in a cell.

A “subject” or “patient” may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. In one embodiment of the methods and compositions provided herein, the mammal is a human.

As used herein, the terms “providing,” “administering,” “introducing,” are used interchangeably herein and refer to the placement of the systems of the disclosure into a subject by a method or route which results in at least partial localization of the system to a desired site. The systems can be administered by any appropriate route which results in delivery to a desired location in the subject.

### 2. SYSTEM FOR TARGETED NUCLEIC ACID DELETIONS

Disclosed herein are systems or kits for targeted nucleic acid deletions comprising: an engineered Clustered Regularly Interspaced Short Palindromic Repeats (CRISPR)-CRISPR associated (Cas) (CRISPR-Cas) system, and/or one or more vectors encoding the engineered CRISPR-Cas system, wherein the engineered CRISPR-Cas system comprises: (a) at least one Cas protein, and (b) a pair of guide RNAs (gRNAs), wherein the pair of gRNAs is configured to hybridize to target sites flanking a nucleic acid sequence for deletion; an engineered transposon system, and/or one or more vectors encoding the engineered transposon system: a recombinase, or catalytic domain thereof, and/or one or more vectors encoding a recombinase, or catalytic domain thereof; and at least one donor nucleic acid to be integrated, wherein the donor nucleic acid comprises a recognition site for the recombinase flanked by at least one transposon end sequence. The system may further comprise a target nucleic acid comprising the nucleic acid sequence for deletion.

The system may be a cell free system. Also disclosed is a cell comprising the system described herein. In some embodiments, the cell is a prokaryotic cell. In some embodiments, the cell is a eukaryotic cell. In some embodiments, the cell is a mammalian cell, a cell of a non-human primate, or a human cell. In some embodiments, the cell is a plant cell.

a. Recombinase

The term “recombinase,” as used herein, refers to a site-specific enzyme that mediates the recombination of DNA between recombinase recognition sequences, which results in the excision, integration, inversion, or exchange (e.g., translocation) of DNA fragments between the recombinase recognition sequences. Recombinases can be classified into two distinct families: serine recombinases (e.g., resolvases and invertases) and tyrosine recombinases (e.g., integrases). Examples of serine recombinases include, without limitation, Hin, Gin, Tn3 (also known as TnpR), β-six, CinH, ParA, γδ, Bxb1, ϕC31, TP901, TG1, ϕBT1, ϕR4, ϕRV1, ϕFC1, MR11, A118, U153, and gp29. Examples of tyrosine recombinases include, without limitation, Cre, FLP, R, Lambda, HK101, HK022, and pSAM2. The serine and tyrosine recombinase names stem from the conserved nucleophilic amino acid residue that the recombinase uses to attack the DNA and which becomes covalently linked to the DNA during strand exchange. The recombinases provided herein are not meant to be exclusive examples of recombinases that can be used in embodiments of the invention. The methods and compositions of the invention can be expanded by mining databases for new orthogonal recombinases or designing synthetic recombinases with defined DNA specificities (See, e.g., Groth et al., “Phage integrases: biology and applications.” J. Mol. Biol. 2004: 335, 667-678; Gordley et al., “Synthesis of programmable integrases.” Proc. Natl. Acad. Sci. USA. 2009; 106, 5053-5058; the entire contents of each are hereby incorporated by reference in their entirety). Other examples of recombinases that are useful in the methods and compositions described herein are known to those of skill in the art, and any new recombinase that is discovered or generated is expected to be able to be used in the different embodiments of the invention. In some embodiments, the recombinase is a serine recombinase. In some embodiments, the recombinase is a tyrosine recombinase.

In some embodiments, the catalytic domains of a recombinase are fused to another protein or provided alone. Recombinases such as this are known, and include those described by Klippel et al., EMBO J. 1988; 7: 3983-3989: Burke et al., Mol Microbiol. 2004; 51: 937-948; Olorunniji et al., Nucleic Acids Res. 2008; 36: 7181-7191; Rowland et al., Mol Microbiol. 2009; 74: 282-298; Akopian et al., Proc Natl Acad Sci USA. 2003; 100: 8688-8691; Gordley et al., J Mol Biol. 2007; 367: 802-813; Gordley et al., Proc Natl Acad Sci USA. 2009; 106: 5053-5058; Arnold et al., EMBO J. 1999; 18: 1407-1414; Gaj et al., “Proc Natl Acad Sci USA. 2011; 108(2):498-503; and Proudfoot et al., PLoS One. 2011; 6(4):e19537; the entire contents of each are hereby incorporated by reference. For example, serine recombinases of the resolvase-invertase group, e.g., Tn3 and γδ resolvases and the Hin and Gin invertases, have modular structures with autonomous catalytic and DNA-binding domains (See, e.g., Grindley et al., Ann Rev Biochem. 2006; 75: 567-605, the entire contents of which are incorporated by reference). The catalytic domains of these recombinases are thus amenable to being in protein fusions. Additionally, many other natural serine recombinases having an N-terminal catalytic domain and a C-terminal DNA binding domain are known (e.g., phiC31 integrase, TnpX transposase, IS607 transposase), and their catalytic domains can be co-opted to engineer programmable site-specific recombinases. Similarly, the core catalytic domains of tyrosine recombinases (e.g., Cre, λ integrase) are known, and can be similarly co-opted to engineer programmable site-specific recombinases as described herein.

In some embodiments, the recombinase comprises Cre recombinase, a mutant, variant, or catalytic domain thereof and the recognition site is a Lox site or variant thereof. In certain embodiments, the Cre recombinase comprises an amino acid sequence of at least 70% identity (e.g., 75%, 80%, 85%, 90%, 95% or 99% identity) to SEQ ID NO: 243. In select embodiments, the Cre recombinase comprises an amino acid sequence of at least 70% identity to SEQ ID NO: 251. In some embodiments, the vector encoding the Cre recombinase comprises a nucleic acid sequence having at least 70% identity to SEQ ID NO: 252 or 253.

SEQ ID NO: 243
PKKKRKVSNLLTVHQNLPALPVDATSDEVRKNLMDMFRDRQAFSEHTWK
MLLSVCRSWAAWCKLNNRKWFPAEPEDVRDYLLYLQARGLAVKTIQQHL
GQLNMLHRRSGLPRPSDSNAVSLVMRRIRKENVDAGERAKQALAFERTD
FDQVRSLMENSDRCQDIRNLAFLGIAYNTLLRIAEIARIRVKDISRTDG
GRMLIHIGRTKTLVSTAGVEKALSLGVTKLVERWISVSGVADDPNNYLF
CRVRKNGVAAPSATSQLSTRALEGIFEATHRLIYGAKDDSGQRYLAWSG
HSARVGAARDMARAGVSIPEIMQAGGWTNVNIVMNYIRNLDSETGAMVR
LLEDGD
SEQ ID NO: 251
MGSSHHHHHHSSGLVPRGSHGGGSAAAMGTRLPKKKRKVSNLLTVHQNL
PALPVDATSDEVRKNLMDMFRDRQAFSEHTWKMLLSVCRSWAAWCKLNN
RKWFPAEPEDVRDYLLYLQARGLAVKTIQQHLGQLNMLHRRSGLPRPSD
SNAVSLVMRRIRKENVDAGERAKQALAFERTDFDQVRSLMENSDRCQDI
RNLAFLGIAYNTLLRIAEIARIRVKDISRTDGGRMLIHIGRTKTLVSTA
GVEKALSLGVTKLVERWISVSGVADDPNNYLFCRVRKNGVAAPSATSQL
STRALEGIFEATHRLIYGAKDDSGQRYLAWSGHSARVGAARDMARAGVS
IPEIMQAGGWTNVNIVMNYIRNLDSETGAMVRLLEDGD

The recognition site for Cre recombinase may include any known Lox sequence or sequence variant. See for example, Missirlis, P I, et al., BMC Genomics, 7:73 (2006), incorporated herein by reference in its entirety. In certain embodiments, the Lox site comprises a nucleic acid sequence of at least 70% identity to SEQ ID NO: 244.

SEQ ID NO: 244
ATAACTTCGTATAGCATACATTATACGAAGTTAT

In some embodiments, the recombinase comprises flippase (FLP) recombinase, a mutant, variant, or catalytic domain thereof and the recognition site is a flippase recognition target (FRT) site or variant thereof. In certain embodiments, the FLP recombinase comprises an amino acid sequence of at least 70% identity (e.g., 75%, 80%, 85%, 90%, 95% or 99% identity) to SEQ ID NO: 245. In some embodiments, the nucleic acid encoding the FLP recombinase comprises a nucleic acid sequence having at least 70% identity to SEQ ID NO: 254.

SEQ ID NO: 245
MPQFDILCKTPPKVLVRQFVERFERPSGEKIALCAAELTYLCWMITHNGT
AIKRATFMSYNTIISNSLSFDIVNKSLQFKYKTQKATILEASLKKLIPAW
EFTIIPYYGQKHQSDITDIVSSLQLQFESSEEADKGNSHSKKMLKALLSE
GESIWEITEKILNSFEYTSRFTKTKTLYQFLFLATFINCGRFSDIKNVDP
KSFKLVQNKYLGVIIQCLVTETKTSVSRHIYFFSARGRIDPLVYLDEFLR
NSEPVLKRVNRTGNSSSNKQEYQLLKDNLVRSYNKALKKNAPYSIFAIKN
GPKSHIGRHLMTSFLSMKGLTELTNVVGNWSDKRASAVARTTYTHQITAI
PDHYFALVSRYYAYDPISKEMIALKDETNPIEEWQHIEQLKGSAEGSIRY
PAWNGNSQEVLDYLSSYINRRI

Several variant FRT sites exist (see Schlake T, et al., Biochemistry 33 (43): 12746-51 (1994), Senecoff J F, et al., Journal of Molecular Biology. 201 (2): 405-21 (1988) and Turan S, et al., Journal of Molecular Biology 402 (1): 52-69 (2010)) and are compatible with the systems and methods described herein. In certain embodiments, the FRT site comprises a nucleic acid sequence of at least 70% identity to SEQ ID NO: 246.

SEQ ID NO: 246
GAAGTTCCTATTCTCTAGAAAGTATAGGAACTTC

In some embodiments, the recombinase comprises TniR resolvase, a mutant, variant, or catalytic domain thereof and the recognition site comprises a resolution (res) sequence or variant thereof. In certain embodiments, the TniR resolvase comprises an amino acid sequence of at least 70% identity (e.g., 75%, 80%. 85%, 90%, 95% or 99% identity) to SEQ ID NO: 247. In some embodiments, the nucleic acid encoding the TniR resolvase comprises a nucleic acid sequence having at least 70% identity to SEQ ID NO: 255.

SEQ ID NO: 247
MLIGYMRVSKADGSQATDLQRDALIAAGVDPVHLYEDQASGMREDRPGL
TSCLKALRTGDTLVVWKLDRLGRDLRHLINTVHDLTGRGIGLKVLTGHG
AAIDTTTAAGKLVFGIFAALAEFERELIAERTIAGLASARARGRKGGRP
FKMTAAKLRLAMAAMGQPETKVGDLCQELGVTRQTLYRHVSPKGELRPD
GEKLLSRI

The sequence of any known TniR res site may be used with the system and methods described herein. In certain embodiments, the res sequence comprises a nucleic acid sequence of at least 70% identity to SEQ ID NO: 248.

SEQ ID NO: 248
CGGCGAGAACTTTCTGGCTCACACTGTCACATAATCGAACGTATATGTG
ACAGGTACGAC

In some embodiments, the recombinase comprises a recombinase from a Tn3-like system (e.g., Tn3 resolvase), also known as TnpR, a mutant, variant, or catalytic domain thereof and the recognition site comprises a resolution (res) sequence or variant thereof. In certain embodiments, the Tn3-like resolvase comprises an amino acid sequence of at least 70% identity (e.g., 75%, 80%, 85%, 90%, 95% or 99% identity) to SEQ ID NO: 249. In some embodiments, the nucleic acid encoding a Tn3 resolvase comprises a nucleic acid sequence having at least 70% identity to SEQ ID NO: 256.

SEQ ID NO: 249
MRLFGYARVSTSQQSLDLQVRALKDAGVKANRIFTDKASGSSTDREGLD
LLRMKVEEGDVILVKKLDRLGRDTADMIQLIKEFDAQGVAVRFIDDGIS
TDGDMGQMVVTILSAVAQAERRRILERTNEGRQEAKLKGIKFGRRRTVD
RNVVLTLHQKGTGATEIAHQLSIARSTVYKILEDERAS

The sequence of any known Tn3-like resolvase res site may be used with the system and methods described herein (See e.g., Grindley N D, et al., Cell 30:19-27 (1982), incorporated herein by reference in its entirety). In certain embodiments, the res sequence comprises a nucleic acid sequence of at least 70% identity to SEQ ID NO: 250.

SEQ ID NO: 250
CCGTACGAAATGTTATAAATTATCGGACATCGTAAAACTGTTACATTAA
TATGTCTATTAAATCGTAAATTTGTAATAATAGACATGAGTTGTCCGAT
ATTCGATTTAAGGTACATTTTT

b. Donor DNA

The donor DNA may be a part of a bacterial plasmid, bacteriophage, plant virus, retrovirus, DNA virus, autonomously replicating extra chromosomal DNA element, linear plasmid, mitochondrial or other organellar DNA, chromosomal DNA, and the like. In some embodiments, the donor nucleic acid comprises a human nucleic acid sequence.

The donor DNA comprises a recognition site for the recombinase, described elsewhere herein, flanked by at least one transposon end sequence. In some embodiments, the donor DNA further comprises a cargo nucleic acid. In some embodiments, the cargo nucleic acid comprises the recognition site for the recombinase. Put another way, the recognition site for the recombinase is within the cargo nucleic acid. The term “transposon end sequence” refers to any nucleic acid comprising a sequence capable of forming a complex with the transposase enzymes thus designating the DNA between the ends, the donor DNA, for rearrangement. Usually, these sequences are inverted repeats about 9 to 40 base pairs long, however the exact sequence requirements differ for the specific transposase enzymes. Transposon end sequences are well known in the art. Transposon ends sequences may or may not include additional sequences that promote or augment transposition.

The donor DNA, and by extension the cargo nucleic acid, may of any suitable length, including, for example, about 50-100 bp (base pairs), about 100-1000 bp, at least or about 10 bp, at least or about 20 bp, at least or about 25 bp, at least or about 30 bp, at least or about 35 bp, at least or about 40 bp, at least or about 45 bp, at least or about 50 bp, at least or about 55 bp, at least or about 60 bp, at least or about 65 bp, at least or about 70 bp, at least or about 75 bp, at least or about 80 bp, at least or about 85 bp, at least or about 90 bp, at least or about 95 bp, at least or about 100 bp, at least or about 200 bp, at least or about 300 bp, at least or about 400 bp, at least or about 500 bp, at least or about 600 bp, at least or about 700 bp, at least or about 800 bp, at least or about 900 bp, at least or about 1 kb (kilobase pair), at least or about 2 kb, at least or about 3 kb, at least or about 4 kb, at least or about 5 kb, at least or about 6 kb, at least or about 7 kb, at least or about 8 kb, at least or about 9 kb, at least or about 10 kb, or less than 10 kb, in length or greater. The donor DNA, and the cargo nucleic acid, may be at least or about 10 kb, at least or about 50 kb, at least or about 100 kb, between 20 kb and 60 kb, between 20 kb and 100 kb.

c. CRISPR-Cas System

In some embodiments, the present system may be derived from a Class 1 (e.g., Type I, Type III, Type VI) or a Class 2 (e.g., Type II, Type V, or Type VI) CRISPR-Cas system. In some embodiments, the present system may be derived from a Type I CRISPR-Cas system. In some embodiments, the present system may be derived from a Type V CRISPR-Cas system.

For example, Type I Cascade complexes may be used in the present methods and systems. Type I CRISPR-Cas systems encode a multi-subunit protein-RNA complex called Cascade, which utilizes a crRNA (or guide RNA) to target double-stranded DNA during an immune response. Cascade itself has no nuclease activity, and degradation of targeted DNA is instead mediated by a trans-acting nuclease known as Cas3. The Type I-F CRISPR-Cas systems and Type I-B CRISPR-Cas systems found within Tn7 transposons consistently lack the Cas3 gene, suggesting that these systems no longer retain any DNA degradation capabilities and have been reduced to RNA-guided DNA-binding complexes. Additionally, one of the core proteins used by Tn7 transposons for selection of DNA target sites for purposes of transposon mobility, TnsD (also known as TniQ), is conspicuously encoded by a gene sitting directly within the Cas gene operon in these systems, suggesting direct coupling or functional relationship between the Cascade complex encoded by Cas genes, and the transpososome enzymatic machinery encoded by Tn seven (Tns) transposase genes.

The system derived from Vibrio cholerae that harbors a Type I-F CRISPR-Cas system may be used with the present system and related methods. Other systems (for which the CRISPR-Cas systems are either categorized as Type I-F or I-B) may also be used with the present system and related methods. These include, without limitation, systems from Vibrio cholerae, Photobacterium iliopiscarium, Pseudoalteromonas sp. P1-25, Pseudoalteromonas ruthenica, Photobacterium ganghwense, Shewanella sp. UCD-KL21, Vibrio diazotrophicus, Vibrio sp. 16, Vibrio sp. F12, Vibrio splendidus, Aliivibrio wodanis, and Parashewanella spongiae.

The Type V systems that encode putative effector gene known as Cas 12k, formerly known as c2c5, may be used in the present methods and systems. The Type V systems encode a putative effector that may be a single protein functioning with a single gRNA. These may have different packaging size, assembly, nuclear localization, etc. Type V CRISPR-Cas systems fall within Class 2 systems, which rely on single-protein effectors together with guide RNA, and so it remains possible that the engineering strategies may be streamlined by using single-protein effectors like Cas12k, rather than the multi-subunit protein-RNA complexes encoded by type I systems, namely Cascade. These operons may be cloned into the same backbones.

The present system may comprise Cas12k. The present system may comprise Cas5, Cas6, Cas7 Cas8, or a combination thereof. In some embodiments, the Cas5 and Cas8 are linked as a functional fusion protein.

d. gRNA

The gRNA may be a crRNA, crRNA/tracrRNA (or single guide RNA, sgRNA).

The terms “gRNA,” “guide RNA” and “CRISPR guide sequence” may be used interchangeably throughout and refer to a nucleic acid comprising a sequence that determines the binding specificity of the CRISPR-Cas system. A gRNA hybridizes to (complementary to, partially or completely) a target nucleic acid sequence (e.g., the genome in a cell). The system may further comprise a target nucleic acid.

The gRNA or portion thereof that hybridizes to the target nucleic acid (a target site) may be any length necessary for selective hybridization. gRNAs or sgRNA(s) used in the present disclosure can be between about 5 and about 100 nucleotides long, or longer (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides in length, or longer). In some embodiments, the gRNA or portion thereof that hybridizes to the target nucleic acid (a target site) may be between 15-40 nucleotides in length. In some embodiments, the gRNA sequence that hybridizes to the target nucleic acid is 15, 16, 17, 18, 19, 20, 21, 22, 23, 24, 25, 26, 27, 28, 29, 30, 31, 32, 33, 34, 35, 36, 37, 38, 39, or 40 nucleotides in length.

The pair of gRNAs may target the same strand, e.g., one target site at the 5′ and one target site at the 3′ end of the nucleic acid sequence for deletion. The pair of gRNAs may target opposite strands of the nucleic acid sequence for deletion. In some embodiments, at least one of the pair of guide RNAs is a non-naturally occurring gRNA. In some embodiments, each of the pair of guide RNAs is a non-naturally occurring gRNA.

To facilitate gRNA design, many computational tools have been developed (See Prykhozhij et al. (PLoS ONE, 10(3): (2015)), Zhu et al. (PLoS ONE, 9(9) (2014)), Xiao et al. (Bioinformatics. Jan. 21 (2014)); Heigwer et al. (Nat Methods, 11(2): 122-123 (2014)). Methods and tools for guide RNA design are discussed by Zhu (Frontiers in Biology, 10 (4) pp 289-296 (2015)), which is incorporated by reference herein. Additionally, there are many publicly available software tools that can be used to facilitate the design of sgRNA(s); including but not limited to, Genscript Interactive CRISPR gRNA Design Tool, WU-CRISPR, and Broad Institute GPP sgRNA Designer. There are also publicly available pre-designed gRNA sequences to target many genes and locations within the genomes of many species (human, mouse, rat, zebrafish, C. elegans), including but not limited to, IDT DNA Predesigned Alt-R CRISPR-Cas9 guide RNAs, Addgene Validated gRNA Target Sequences, and GenScript Genome-wide gRNA databases.

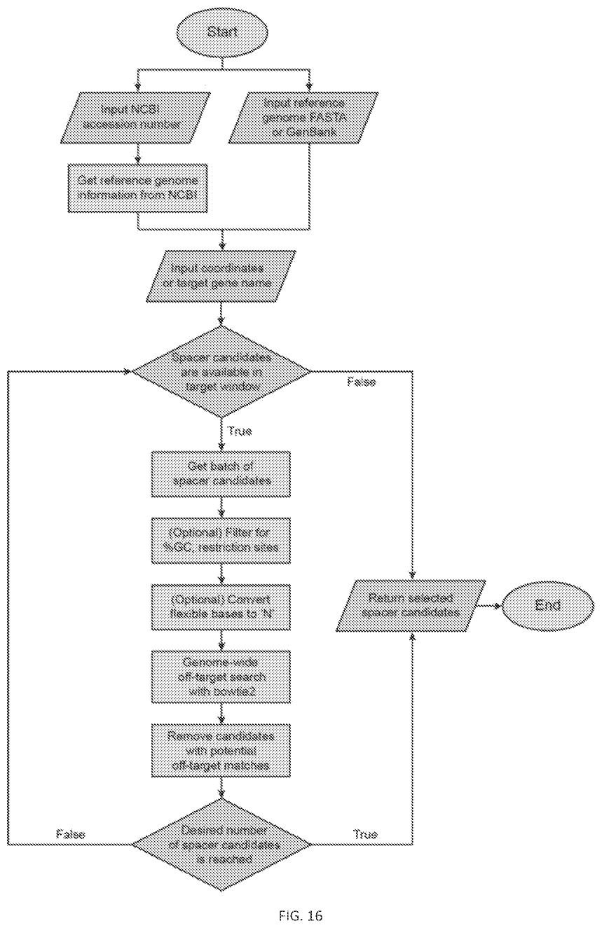

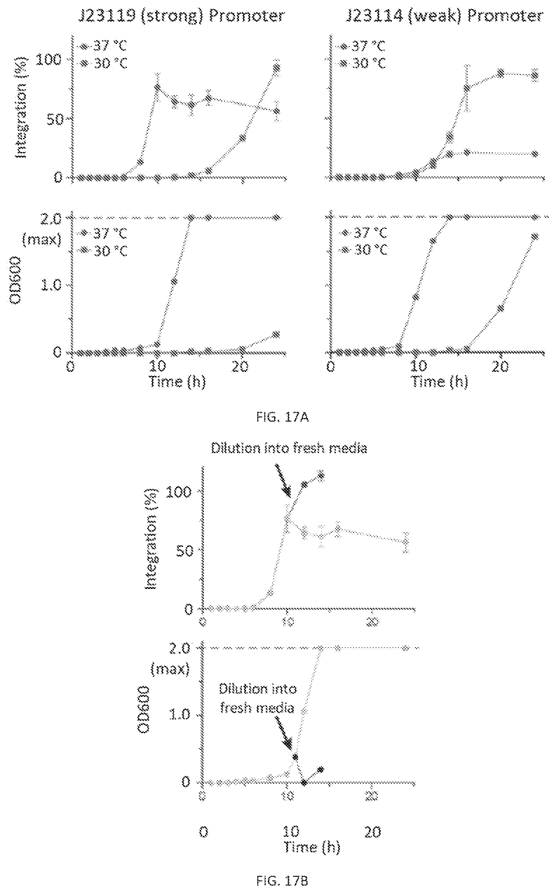

In some embodiments, an exemplary guide RNA design algorithm is as shown in FIG. 16. Based on a target nucleic acid (e.g., target gene name or genomic coordinates), spacers with a defined length and PAM are generated and filtered from a given reference genome. An alignment tool is used to evaluate each spacer candidate for potential off-targets genome-wide, as determined by a less than a user-specified maximum mismatch limit. The program is capable of converting flexible bases, e.g., which do not contribute to spacer-protospacer complementarity, to ‘N’ to exclude these bases from contributing to the mismatch count for the genome-wide off-target search. The off-target search module can also be executed separately for the evaluation of user-specified spacers.

In addition to a sequence that binds to a target nucleic acid, in some embodiments, the gRNA may also comprise a scaffold sequence (e.g., tracrRNA). In some embodiments, such a chimeric gRNA may be referred to as a single guide RNA (sgRNA). Exemplary scaffold sequences will be evident to one of skill in the art and can be found, for example, in Jinek, et al. Science (2012) 337(6096):816-821, and Ran, et al. Nature Protocols (2013) 8:2281-2308, incorporated herein by reference in their entireties.

In some embodiments, the gRNA sequence does not comprise a scaffold sequence and a scaffold sequence is expressed as a separate transcript. In such embodiments, the gRNA sequence further comprises an additional sequence that is complementary to a portion of the scaffold sequence and functions to bind (hybridize) the scaffold sequence.

In some embodiments, the gRNA sequence is at least 50%, 55%, 60%, 65%, 70%, 75%, 80%. 85%, 90%, 95%. 96%, 97%, 98%. 99%, or at least 100% complementary to a target nucleic acid. In some embodiments, the gRNA sequence is at least 50%, 55%, 60%, 65%, 70%, 75%, 80%, 85%, 90%, 95%, 96%, 97%, 98%, 99%, or at least 100% complementary to the 3′ end of the target nucleic acid (e.g., the last 5, 6, 7, 8, 9, or 10 nucleotides of the 3′ end of the target nucleic acid).

The gRNA may be a non-naturally occurring gRNA.

The target sequence may or may not be flanked by a protospacer adjacent motif (PAM) sequence. In some embodiments, the target nucleic acid is flanked by a protospacer adjacent motif (PAM). A PAM site is a nucleotide sequence in proximity to a target sequence. For example, PAM may be a DNA sequence immediately following the DNA sequence targeted by the CRISPR-Cas system. Pam sequences are well-known in the art. Non-limiting examples of the PAM sequences include: CC, CA, AG, GT, TA, AC, CA, GC, CG, GG, CT, TG, GA, AGG, TGG, T-rich PAMs (such as TTT, TTG, TTC, etc.), NGG, NGA, NAG, and NGGNG, where “N” is any nucleotide.

In certain embodiments, a nucleic acid-guided nuclease can only cleave a target sequence if an appropriate PAM is present. See, for example Doudna et al., Science, 2014, 346(6213): 1258096, incorporated herein by reference. A PAM can be 5′ or 3′ of a target sequence. A PAM can be upstream or downstream of a target sequence. In one embodiment, the target sequence is immediately flanked on the 3′ end by a PAM sequence. A PAM can be 1, 2, 3, 4, 5, 6, 7, 8, 9, 10 or more nucleotides in length. In certain embodiments, a PAM is between 2-6 nucleotides in length. The target sequence may or may not be located adjacent to a PAM sequence (e.g., PAM sequence located immediately 3′ of the target sequence). In some embodiments, e.g., Type I systems, the PAM is on the alternate side of the protospacer (the 5′ end). Makarova et al. describes the nomenclature for all the classes, types, and subtypes of CRISPR systems (Nature Reviews Microbiology 13:722-736 (2015)). Guide structures and PAMs are described in by R. Barrangou (Genome Biol. 16:247 (2015)).

“Complementarity” refers to the ability of a nucleic acid to form hydrogen bond(s) with another nucleic acid sequence by either traditional Watson-Crick or other non-traditional types. A percent complementarity indicates the percentage of residues in a nucleic acid molecule, which can form hydrogen bonds (e.g., Watson-Crick base pairing) with a second nucleic acid sequence. Full complementarity is not necessarily required, provided there is sufficient complementarity to cause hybridization. There may be mismatches distal from the PAM.

e. Transposon System

An engineered transposon system of the present invention may comprise one or more transposases or other components of a transposon. The engineered transposon system facilitates cleavage of the target nucleic acid and subsequent insertion of the donor nucleic acid into the target nucleic acid. The engineered transposon system of the present invention may be derived from any of the known transposon systems and/or transposon components. The transposon systems and components may have different efficiency, different specificity, different transposon end sequences, and the like, but retain the capability to facilitate cleavage of the target nucleic acid and subsequent insertion of the donor nucleic acid into the target nucleic acid.

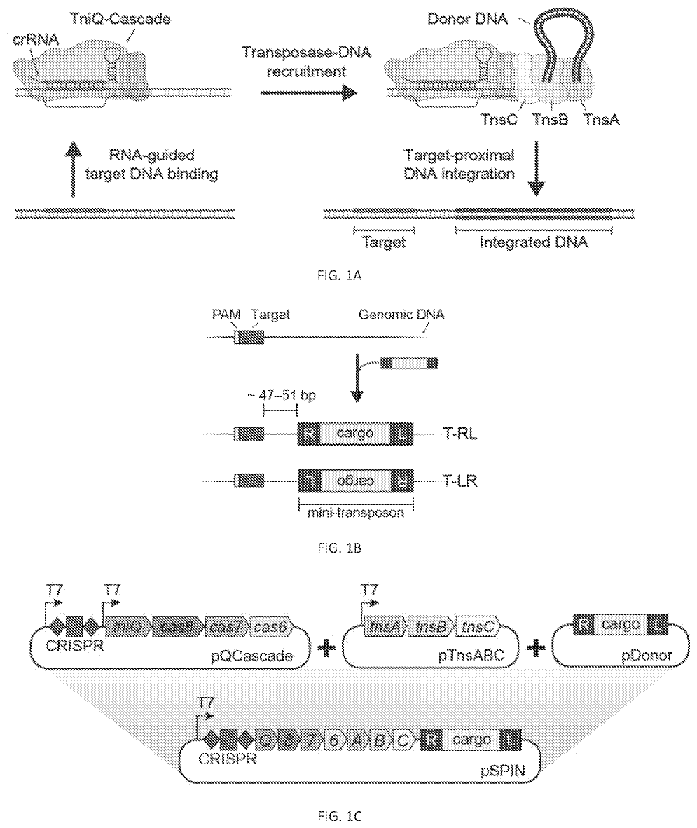

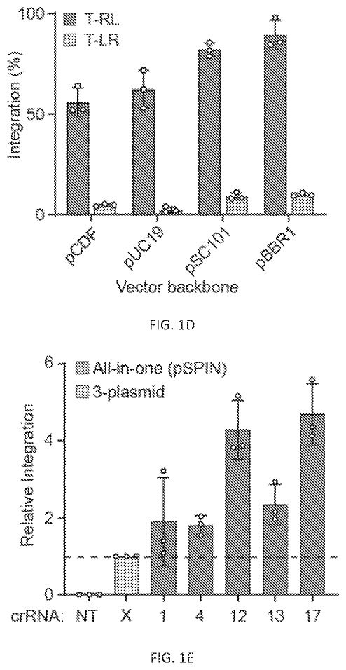

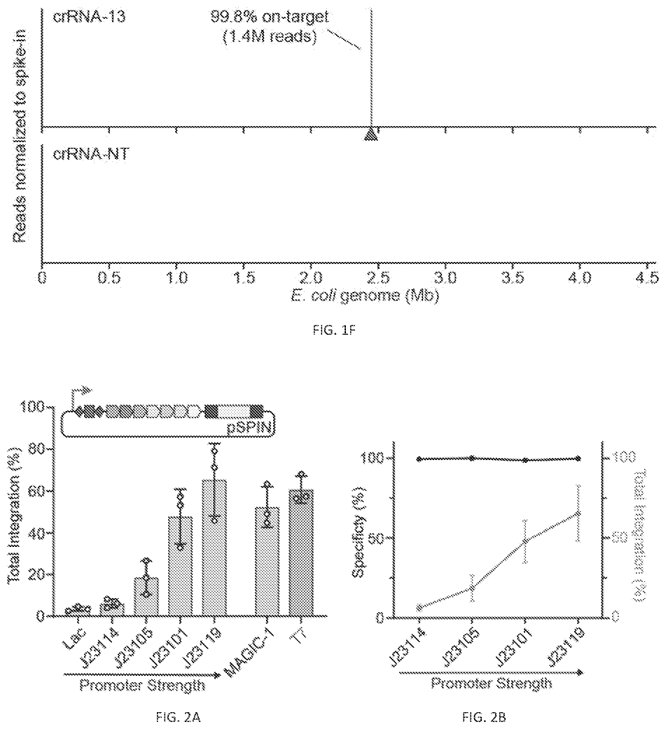

In some embodiments, the transposon is a Tn7 or Tn7-like transposon. The Tn7 transposon contains characteristic left and right end sequences and encodes five tns genes, mnsA E, which collectively encode a heteromeric transposase. TnsA is a catalytic enzyme that excises the transposon donor via coordinated double-strand breaks with TnsB. Catalytically impaired TnsA mutants still facilitated genetic modification and may be suitable for the systems and methods disclosed herein. Tn7 and Tn7-like transposons may be categorized based on the presence of the hallmark DDE-like transposase gene, tnsB (also referred to as tniA), the presence of a gene encoding a protein within the AAA+ ATPase family, tnsC (also referred to as tniB), one or more targeting factors that define integration sites (which may include a protein within the tniQ family, also referred to as tnsD, but sometimes includes other distinct targeting factors), and inverted repeat transposon ends that typically comprise multiple binding sites thought to be specifically recognized by the TnsB transposase protein. In Tn7, the targeting factors, or “target selectors,” comprise the genes tnsD and tnsE. Based on biochemical and genetics studies, it is known that TnsD binds a conserved attachment site in the 3′ end of the glmS gene, directing downstream integration, whereas TnsE binds the lagging strand replication fork and directs sequence-non-specific integration primarily into replicating/mobile plasmids. Thus, Tn7 exhibits mobilization patterns that allow for both horizontal and vertical spread (FIG. 1A). In some embodiments, the transposon system comprises TnsA, TnsB, TnsC, or a combination thereof.

The most well-studied member of this family of transposons is Tn7, hence why the broader family of transposons may be referred to as Tn7-like. “Tn7-like” term does not imply any particular evolutionary relationship between Tn7 and related transposons: in some cases, a Tn7-like transposon will be even more basal in the phylogenetic tree and thus Tn7 can be considered as having evolved from, or derived from, this related Tn7-like transposon.

Whereas Tn7 comprises tnsD and tnsE target selectors, related transposons comprise other genes for targeting: for example, Tn5090/Tn5053 encode a member of the tniQ family (a homolog of E. coli tnsD) as well as a resolvase gene tniR (see below): Tn6230 encodes the protein TnsF; and Tn6022 encodes two uncharacterized open reading frames orf2 and orf3; Tn6677 and related transposons encode variant Type I-F and Type I-B CRISPR-Cas systems that work together with TniQ for RNA-guided mobilization; and other transposons encode Type V-U5 CRISPR-Cas systems that work together with TniQ for random and RNA-guided mobilization. Any of the above transposon systems are compatible with the systems and methods described herein. In some embodiments, the transposons system comprises TniQ.

The present system might comprise the transposon Tn6677 in combination with a variant Type I-F CRISPR-Cas (See, Klompe et al., Nature 571, 219-225 (2019) and International Patent Application No. PCT/US20/21568, each incorporated herein by reference in their entirety). The transposon-associated genes comprise tnsA-tnsB-tnsC as well as the tniQ gene that is in the same operon as cas8-cas7-cas6. The transposon Tn6677 may be derived from a Vibrio cholerae or other applicable species, for example those disclosed in International Patent Application No. PCT/US20/21568, incorporated herein by reference in its entirety.

A type V-K CRISPR-Cas system was shown to direct RNA-guided transposition, though a considerable degree of random integration still occurred in this system. The CRISPR-Cas machinery comprises the Cas12k protein and a dual-guide RNA (which could be fused into a single chimeric guide RNA, or sgRNA); the transposon-associated genes comprise tnsB-tnsC-tniQ. The transposon may be derived from a Scytonema hofmanni isolate. The present system might comprise the transposon comprising tnsB-tnsC-tniQ, e.g., as derived from Scytonema hofmanni, or other homologous transposons, in combination with a variant Type V-K CRISPR-Cas system.

f. Vectors

The engineered CRISPR-Cas system and the engineered transposon system may be on the same or different vector(s). The recombinase, or catalytic domain thereof, may be on the same or different vector(s) from either the CRISPR-Cas system and/or the transposon system. For example, the system described herein can be employed through expression of the recombinase in trans. The present system can be delivered to a subject or cell using one or more vectors (e.g., 1, 2, 3, 4, 5, 6, 7, 8, 9, or more vectors). One or more gRNAs (e.g., sgRNAs) can be in a single (one) vector or two or more vectors. The vector may also include the donor nucleic acid. One or more Cas proteins and/or transposon proteins and/or recombinase and/or gRNAs and/or donor nucleic acid can be in the same, or separate vectors. The present disclosure further provides engineered, non-naturally occurring vectors and vector systems, which can encode one or more components of the present system.

Vectors can be administered directly to patients (in vivo) or they can be used to manipulate cells in vitro or ex vivo, where the modified cells may be administered to patients. The vectors of the present disclosure may be delivered to a eukaryotic cell in a subject. Modification of the eukaryotic cells via the present system can take place in a cell culture, where the method comprises isolating the eukaryotic cell from a subject prior to the modification. In some embodiments, the method further comprises returning said eukaryotic cell and/or cells derived therefrom to the subject.

Conventional viral and non-viral based gene transfer methods can be used to introduce nucleic acids encoding components of the present system into cells, tissues, or a subject. Such methods can be used to administer nucleic acids encoding components of the present system to cells in culture, or in a host organism. Non-viral vector delivery systems include DNA plasmids, cosmids, RNA (e.g., a transcript of a vector described herein), a nucleic acid, and a nucleic acid complexed with a delivery vehicle. Viral vector delivery systems include DNA and RNA viruses, which have either episomal or integrated genomes after delivery to the cell. Viral vectors include, for example, retroviral, lentiviral, adenoviral, adeno-associated and herpes simplex viral vectors.

In certain embodiments, the requisite protein and nucleic acid components may be expressed on the same plasmid as the donor nucleic acid, so that the entire system is fully autonomous. The protein and nucleic acid components guiding the targeting and deletion may be encoded within the donor nucleic acid (e.g., the cargo nucleic acid), such that it can guide further mobilization autonomously, whether in the originally transformed microbe, or in other microbes (e.g., in a conjugative plasmid context, in a microbiome context, etc.).

In certain embodiments, the requisite protein and nucleic acid (e.g., gRNAs, donor nucleic acid) components may be expressed on two or more plasmids.

Promoters that may be used include T7 RNA polymerase promoters, constitutive E. coli promoters, and promoters that could be broadly recognized by transcriptional machinery in a wide range of bacterial organisms. The system may be used with various bacterial hosts.

In certain embodiments, plasmids that are non-replicative, or plasmids that can be cured by high temperature may be used. The donor nucleic acid, and donor nucleic acid/CRISPR-associated components, may be removed from the engineered cells under certain conditions. This may allow for nucleic acid deletions by transforming bacteria of interest, but then being left with engineered strains that have no memory of the plasmids used to facilitate the modification.

Drug selection strategies may be adopted for positively selecting for cells that underwent targeted nucleic acid deletion. The donor nucleic acid may contain one or more drug-selectable markers within a cargo. Then presuming that the original donor nucleic acid plasmid or vector having the other components of the system is removed, drug selection may be used to enrich for integrated clones. Colony screenings may be used to isolate clonal events.

A variety of viral constructs may be used to deliver the present system (such as one or more Cas proteins and/or Tns proteins, gRNA(s), donor DNA, etc.) to the targeted cells and/or a subject. Nonlimiting examples of such recombinant viruses include recombinant adeno-associated virus (AAV), recombinant adenoviruses, recombinant lentiviruses, recombinant retroviruses, recombinant herpes simplex viruses, recombinant poxviruses, phages, etc. The present disclosure provides vectors capable of integration in the host genome, such as retrovirus or lentivirus. See, e.g., Ausubel et al., Current Protocols in Molecular Biology, John Wiley & Sons, New York, 1989; Kay, M. A., et al., 2001 Nat. Medic. 7(1):33-40; and Walther W. and Stein U., 2000 Drugs, 60(2): 249-71, incorporated herein by reference.

The present disclosure also provides for DNA segments encoding the proteins disclosed herein, vectors containing these segments and host cells containing the vectors. The vectors may be used to propagate the segment in an appropriate host cell and/or to allow expression from the segment (e.g., an expression vector). The person of ordinary skill in the art would be aware of the various vectors available for propagation and expression of a cloned DNA sequence. In one embodiment, a DNA segment encoding the present protein(s) is contained in a plasmid vector that allows expression of the protein(s) and subsequent isolation and purification of the protein produced by the recombinant vector. Accordingly, the proteins disclosed herein can be purified following expression from the native transposon, obtained by chemical synthesis, or obtained by recombinant methods.

To construct cells that express the present system, expression vectors for stable or transient expression of the present system may be constructed via conventional methods as described herein and introduced into host cells. For example, nucleic acids encoding the components of the present system may be cloned into a suitable expression vector, such as a plasmid or a viral vector in operable linkage to a suitable promoter. The selection of expression vectors/plasmids/viral vectors should be suitable for integration and replication in eukaryotic cells.

In certain embodiments, vectors of the present disclosure can drive the expression of one or more sequences in mammalian cells using a mammalian expression vector. Examples of mammalian expression vectors include pCDM8 (Seed, Nature (1987) 329:840, incorporated herein by reference) and pMT2PC (Kaufman, et al., EMBO J. (1987) 6:187, incorporated herein by reference). When used in mammalian cells, the expression vector's control functions are typically provided by one or more regulatory elements. For example, commonly used promoters are derived from polyoma, adenovirus, cytomegalovirus, simian virus, and others disclosed herein and known in the art. For other suitable expression systems for both prokaryotic and eukaryotic cells see, e.g., Chapters 16 and 17 of Sambrook, et al., MOLECULAR CLONING: A LABORATORY MANUAL. 2nd eds., Cold Spring Harbor Laboratory, Cold Spring Harbor Laboratory Press, Cold Spring Harbor, N.Y., 1989, incorporated herein by reference.

Vectors of the present disclosure can comprise any of a number of promoters known to the art, wherein the promoter is constitutive, regulatable or inducible, cell type specific, tissue-specific, or species specific. In addition to the sequence sufficient to direct transcription, a promoter sequence of the invention can also include sequences of other regulatory elements that are involved in modulating transcription (e.g., enhancers, Kozak sequences and introns). Many promoter/regulatory sequences useful for driving constitutive expression of a gene are available in the art and include, but are not limited to, for example, CMV (cytomegalovirus promoter), EF1a (human elongation factor 1 alpha promoter), SV40 (simian vacuolating virus 40 promoter), PGK (mammalian phosphoglycerate kinase promoter), Ubc (human ubiquitin C promoter), human beta-actin promoter, rodent beta-actin promoter, CBh (chicken beta-actin promoter), CAG (hybrid promoter contains CMV enhancer, chicken beta actin promoter, and rabbit beta-globin splice acceptor), TRE (Tetracycline response element promoter), H1 (human polymerase III RNA promoter), U6 (human U6 small nuclear promoter), and the like. Additional promoters that can be used for expression of the components of the present system, include, without limitation, cytomegalovirus (CMV) intermediate early promoter, a viral LTR such as the Rous sarcoma virus LTR, HIV-LTR, HTLV-1 LTR, Maloney murine leukemia virus (MMLV) LTR, myeoloproliferative sarcoma virus (MPSV) LTR, spleen focus-forming virus (SFFV) LTR, the simian virus 40 (SV40) early promoter, herpes simplex tk virus promoter, elongation factor 1-alpha (EF1-α) promoter with or without the EF1-α intron. Additional promoters include any constitutively active promoter. Alternatively, any regulatable promoter may be used, such that its expression can be modulated within a cell.

Moreover, inducible and tissue specific expression of a RNA, transmembrane proteins, or other proteins can be accomplished by placing the nucleic acid encoding such a molecule under the control of an inducible or tissue specific promoter/regulatory sequence. Examples of tissue specific or inducible promoter/regulatory sequences which are useful for this purpose include, but are not limited to, the rhodopsin promoter, the MMTV LTR inducible promoter, the SV40 late enhancer/promoter, synapsin 1 promoter, ET hepatocyte promoter, GS glutamine synthase promoter and many others. Various commercially available ubiquitous as well as tissue-specific promoters and tumor-specific are available, for example from InvivoGen. In addition, promoters which are well known in the art can be induced in response to inducing agents such as metals, glucocorticoids, tetracycline, hormones, and the like, are also contemplated for use with the invention. Thus, it will be appreciated that the present disclosure includes the use of any promoter/regulatory sequence known in the art that is capable of driving expression of the desired protein operably linked thereto.

The vectors of the present disclosure may direct expression of the nucleic acid in a particular cell type (e.g., tissue-specific regulatory elements are used to express the nucleic acid). Such regulatory elements include promoters that may be tissue specific or cell specific. The term “tissue specific” as it applies to a promoter refers to a promoter that is capable of directing selective expression of a nucleotide sequence of interest to a specific type of tissue (e.g., seeds) in the relative absence of expression of the same nucleotide sequence of interest in a different type of tissue. The term “cell type specific” as applied to a promoter refers to a promoter that is capable of directing selective expression of a nucleotide sequence of interest in a specific type of cell in the relative absence of expression of the same nucleotide sequence of interest in a different type of cell within the same tissue. The term “cell type specific” when applied to a promoter also means a promoter capable of promoting selective expression of a nucleotide sequence of interest in a region within a single tissue. Cell type specificity of a promoter may be assessed using methods well known in the art, e.g., immunohistochemical staining.

Additionally, the vector may contain, for example, some or all of the following: a selectable marker gene, such as the neomycin gene for selection of stable or transient transfectants in host cells; enhancer/promoter sequences from the immediate early gene of human CMV for high levels of transcription; transcription termination and RNA processing signals from SV40 for mRNA stability: 5′- and 3′-untranslated regions for mRNA stability and translation efficiency from highly-expressed genes like α-globin or β-globin; SV40 polyoma origins of replication and ColE1 for proper episomal replication; internal ribosome binding sites (IRESes), versatile multiple cloning sites; T7 and SP6 RNA promoters for in vitro transcription of sense and antisense RNA: a “suicide switch” or “suicide gene” which when triggered causes cells carrying the vector to die (e.g., HSV thymidine kinase, an inducible caspase such as iCasp9), and reporter gene for assessing expression of the chimeric receptor. Suitable vectors and methods for producing vectors containing transgenes are well known and available in the art. Selectable markers also include chloramphenicol resistance, tetracycline resistance, spectinomycin resistance, streptomycin resistance, erythromycin resistance, rifampicin resistance, bleomycin resistance, thermally adapted kanamycin resistance, gentamycin resistance, hygromycin resistance, trimethoprim resistance, dihydrofolate reductase (DHFR), GPT: the URA3, HIS4, LEU2, and TRP1 genes of S. cerevisiae.

When introduced into the host cell, the vectors may be maintained as an autonomously replicating sequence or extrachromosomal element or may be integrated into host DNA.

In one embodiment, the donor nucleic acid may be delivered using the same gene transfer system as used to deliver the Cas protein, the recombinase, and/or transposon system proteins (included on the same vector) or may be delivered using a different delivery system. In another embodiment, the donor nucleic acid may be delivered using the same transfer system as used to deliver gRNA(s).

In one embodiment, the present disclosure comprises integration of an exogenous nucleic acid into the endogenous gene. Alternatively, an exogenous nucleic acid is not integrated into the endogenous gene. The donor nucleic acid may be packaged into an extrachromosomal, or episomal vector (such as AAV vector), which persists in the nucleus in an extrachromosomal state, and offers donor-template delivery and expression without integration into the host genome. Use of extrachromosomal gene vector technologies has been discussed in detail by Wade-Martins R (Methods Mol Biol. 2011; 738:1-17, incorporated herein by reference).

The present system (e.g., proteins, polynucleotides encoding these proteins, nucleic acids and compositions comprising the proteins and/or polynucleotides described herein) may be delivered by any suitable means. In certain embodiments, the system is delivered in vivo. In other embodiments, the system is delivered to isolated/cultured cells (e.g., autologous iPS cells) in vitro to provide modified cells useful for in vivo delivery to patients afflicted with a disease or condition.

Vectors according to the present disclosure can be transformed, transfected, or otherwise introduced into a wide variety of host cells. Transfection refers to the taking up of a vector by a host cell whether or not any coding sequences are in fact expressed. Numerous methods of transfection are known to the ordinarily skilled artisan, for example, lipofectamine, calcium phosphate co-precipitation, electroporation, DEAE-dextran treatment, microinjection, viral infection, and other methods known in the art. Transduction refers to entry of a virus into the cell and expression (e.g., transcription and/or translation) of sequences delivered by the viral vector genome. In the case of a recombinant vector, “transduction” generally refers to entry of the recombinant viral vector into the cell and expression of a nucleic acid of interest delivered by the vector genome.

Any of the vectors comprising a nucleic acid sequence that encodes the components of the present system is also within the scope of the present disclosure. Such a vector may be delivered into cells by a suitable method. Methods of delivering vectors to cells are well known in the art and may include DNA or RNA electroporation, transfection reagents such as liposomes or nanoparticles to delivery DNA or RNA; delivery of DNA, RNA, or protein by mechanical deformation (see. e.g., Sharei et al. Proc. Natl. Acad. Sci. USA (2013) 110(6): 2082-2087, incorporated herein by reference); or viral transduction. In some embodiments, the vectors are delivered to cells by viral transduction. Nucleic acids can be delivered as part of a larger construct, such as a plasmid or viral vector, or directly, e.g., by electroporation, lipid vesicles, viral transporters, microinjection, and biolistics (high-speed particle bombardment). Similarly, the construct containing the one or more transgenes can be delivered by any method appropriate for introducing nucleic acids into a cell. In some embodiments, the construct or the nucleic acid encoding the components of the present system is a DNA molecule. In some embodiments, the nucleic acid encoding the components of the present system is a DNA vector and may be electroporated to cells. In some embodiments, the nucleic acid encoding the components of the present system is an RNA molecule, which may be electroporated to cells.

Additionally, delivery vehicles such as nanoparticle- and lipid-based mRNA or protein delivery systems can be used. Further examples of delivery vehicles include lentiviral vectors, ribonucleoprotein (RNP) complexes, lipid-based delivery system, gene gun, hydrodynamic, electroporation or nucleofection microinjection, and biolistics. Various gene delivery methods are discussed in detail by Nayerossadat et al. (Adv Biomed Res. 2012; 1: 27) and Ibraheem et al. (Int J Pharm. 2014 Jan. 1; 459(1-2):70-83), incorporated herein by reference.

Exemplary vectors encoding the systems described herein are provided in SEQ ID NO: 15-38 and additional vectors appropriate for the methods and uses described herein may be found in International Application No. PCT/US20/21568.

### 3. METHODS

Also disclosed herein are methods for deleting a nucleic acid sequence from a target nucleic acid and methods of inactivating a gene of interest using the disclosed systems or kits. Further disclosed are methods for genetically modifying diverse bacterial communities (e.g., gut or fecal-derived bacteria).

Methods for deleting a nucleic acid sequence of interest from a target nucleic acid comprise contacting the target nucleic acid with the system described herein. The methods can be used to delete any nucleic acid sequence of interest from a target nucleic acid. The methods may be used in vitro, ex vivo, or in vivo.

In some embodiments, the nucleic acid sequence of interest acid is chromosomal DNA or genomic DNA. In some embodiments, the nucleic acid sequence of interest is bacterial plasmid DNA. The nucleic acid sequence of interest can comprise portion of or an entire gene (e.g., the promoter region, the coding region, the termination region, or any combination thereof). In some embodiments, the nucleic acid sequence of interest comprises non-coding DNA. The nucleic acid sequence of interest can comprise regions which are responsible for producing RNA.

The nucleic acid sequence of interest can be of any size. For example, the nucleic acid sequence of interest may be 10 bases or 100 kilobases. In some embodiments, the nucleic acid sequence of interest comprises at least 50 bases, at least 100 bases, at least 1 kilobase, at least 5 kilobases, at least, 10 kilobases, at least 15 kilobases, or at least 20 kilobases.

The descriptions and embodiments provided above for the engineered CRISPR-Cas system, the engineered transposon system, the recombinase, or catalytic domain thereof, and the donor nucleic acid are applicable to the methods described herein.

In some embodiments, the methods may comprise introducing the disclosed systems into a cell. In some embodiments, the recombinase, or catalytic domain thereof, and/or one or more vectors encoding a recombinase, or catalytic domain thereof is introduced to the cell after the introduction of the engineered CRISPR-Cas system, the engineered transposon system, and the at least one donor nucleic acid. For example, all four components may be introduced simultaneous or nearly simultaneously. In some embodiments, all four components may be introduced, in any order, with a time period separating each introduction. In alternative embodiments, the introduction of the recombinase to the cell is after the introduction the CRISPR-Cas system, the transposon system, and the donor nucleic acid, such that RNA-guided nucleic acid integration has already occurred.

Methods for inactivating a gene of interest comprise introducing into one or more cells the systems described herein, wherein the nucleic acid sequence for deletion comprises at least a portion of the gene of interest. The one or more cells may be eukaryotic cells or prokaryotic cells.

The gene of interest may comprise any gene of interest to inactivate or delete. In some embodiments, the gene of interest comprises an antibiotic resistance gene, a virulence gene, a metabolic gene, a toxin gene, a remodeling gene, a gene or gene variant responsible for a disease, or a mutant gene.

In some embodiments, the gene of interest is located chromosomally. In some embodiments, the gene of interest is located episomally, e.g., in bacterial cells.

The cell can be a mitotic and/or post-mitotic cell from any eukaryotic cell or organism (e.g. a cell of a single-cell eukaryotic organism, a plant cell, an algal cell, a fungal cell (e.g., a yeast cell), an animal cell, a cell from an invertebrate animal (e.g. fruit fly, cnidarian, echinoderm, nematode, an insect, an arachnid, etc.), a cell from a vertebrate animal (e.g., fish, amphibian, reptile, bird, mammal), a cell from a mammal, a cell from a rodent, a cell from a human, etc.), or a protozoan cell. Any type of cell may be of interest (e.g. a stem cell, e.g. an embryonic stem (ES) cell, an induced pluripotent stem (iPS) cell, a germ cell; a somatic cell, e.g. a fibroblast, a hematopoietic cell, a neuron, a muscle cell, a bone cell, a hepatocyte, a pancreatic cell, a liver cell, a lung cell, a skin cell: an in vitro or in vivo embryonic cell of an embryo at any stage, e.g., a 1-cell, 2-cell, 4-cell, 8-cell, etc. stage zebrafish embryo; etc.). Cells may be from established cell lines or they may be primary cells, where “primary cells”, “primary cell lines”, and “primary cultures” are used interchangeably herein to refer to cells and cells cultures that have been derived from a subject and allowed to grow in vitro for a limited number of passages.

In some embodiments, the one or more cells comprise plant cells. Suitable plant cells may be from a number of different plants including, but are not limited to, monocotyledonous and dicotyledonous plants, such as crops including grain crops (e.g., wheat, maize, rice, millet, barley), fruit crops (e.g., tomato, apple, pear, strawberry, orange), forage crops (e.g., alfalfa), root vegetable crops (e.g., carrot, potato, sugar beets, yam), leafy vegetable crops (e.g., lettuce, spinach); flowering plants (e.g., petunia, rose, chrysanthemum), conifers and pine trees (e.g., pine fir, spruce): plants used in phytoremediation (e.g., heavy metal accumulating plants); oil crops (e.g., sunflower, rapeseed) and plants used for experimental purposes (e.g., Arabidopsis). Thus, the disclosed methods and compositions have use over a broad range of plants, including, but not limited to, species from the genera Asparagus, Avena, Brassica, Citrus, Citrullus, Capsicum, Cucurbita, Daucus, Glycine, Hordeum, Lactuca, Lycopersicon, Malus, Manihot, Nicotiana, Oryza, Persea, Pisum, Pyrus, Prunus, Raphanus, Secale, Solanum, Sorghum, Triticum, Vitis, Vigna, and Zea.

In some embodiments, the one or more cells are animal cells. The present disclosure provides for a modified animal cell produced by the present system and method, an animal comprising the animal cell, a population of cells comprising the cell, tissues, and at least one organ of the animal. The present disclosure further encompasses the progeny, clones, cell lines or cells of the genetically modified animal. The present cells may be used for transplantation (e.g., hematopoietic stem cells or bone marrow).

Non-limiting examples of animal cells that may be genetically modified using the systems and methods include, but are not limited to, cells from: mammals such as primates (e.g., ape, chimpanzee, macaque), rodents (e.g., mouse, rabbit, rat), canine or dog, livestock (cow/bovine, donkey, sheep/ovine, goat or pig), fowl or poultry (e.g., chicken), and fish (e.g., zebra fish). The present methods and systems may be used for cells from other eukaryotic model organisms, e.g., Drosophila, C. elegans, etc. In certain embodiments, the mammal is a human, a non-human primate (e.g., marmoset, rhesus monkey, chimpanzee), a rodent (e.g., mouse, rat, gerbil, Guinea pig, hamster, cotton rat, naked mole rat), a rabbit, a livestock animal (e.g., goat, sheep, pig, cow, cattle, buffalo, horse, camelid), a pet mammal (e.g., dog, cat), a zoo mammal, a marsupial, an endangered mammal, and an outbred or a random bred population thereof.

In some embodiments, the one or more cells comprise microbial cells. In some embodiments, the microbial cells are Gram-negative bacterial cells, Gram-positive bacterial cells, or a combination thereof. In some embodiments, the microbial cells are pathogenic bacterial cells. In some embodiments, the microbial cells are non-pathogenic bacterial cells (e.g., probiotic and/or commensal bacterial cells). In some embodiments, the microbial cells form microbial flora (e.g., natural human microbial flora). In some embodiments, the microbial cells are used in industrial or environmental bioprocesses (e.g., bioremediation).

The cell can be a cancer cell. The cell can be a stem cell. Examples of stem cells include pluripotent, multipotent and unipotent stem cells. Examples of pluripotent stem cells include embryonic stem cells, embryonic germ cells, embryonic carcinoma cells and induced pluripotent stem cells (iPSCs). The cell may be an induced pluripotent stem cell (iPSC), e.g., derived from a fibroblast of a subject. In another embodiment, the cell can be a fibroblast.

Cell replacement therapy can be used to prevent, correct, or treat a disease or condition, where the methods of the present disclosure are applied to isolated patient's cells (er vivo), which is then followed by the administration of the genetically modified cells into the patient.

The cell may be autologous or allogeneic to the subject who is administered the cell. As described herein, the genetically modified cells may be autologous to the subject, e.g., the cells are obtained from the subject in need of the treatment, genetically engineered, and then administered to the same subject. Alternatively, the host cells are allogeneic cells, e.g., the cells are obtained from a first subject, genetically engineered, and administered to a second subject that is different from the first subject but of the same species. In some embodiments, the genetically modified cells are allogeneic cells and have been further genetically engineered to reduced graft-versus-host disease.

“Induced pluripotent stem cells.” commonly abbreviated as iPS cells or iPSCs, refer to a type of pluripotent stem cell artificially prepared from a non-pluripotent cell, typically an adult somatic cell, or terminally differentiated cell, such as a fibroblast, a hematopoietic cell, a myocyte, a neuron, an epidermal cell, or the like, by introducing certain factors, referred to as reprogramming factors.

The term “autologous” refers to any material derived from the same individual to whom it is later to be re-introduced into the same individual.

The term “allogeneic” refers to any material derived from a different animal of the same species as the individual to whom the material is introduced. Two or more individuals of the same species are said to be allogeneic to one another.

The systems and methods may be used to modify a stem cell. The term “stem cell” is used herein to refer to a cell that has the ability both to self-renew and to generate a differentiated cell type (see Morrison et al. (1997) Cell 88:287-298, incorporated herein by reference). Stem cells may be characterized by both the presence of specific markers (e.g., proteins, RNAs, etc.) and the absence of specific markers. Stem cells may also be identified by functional assays both in vitro and in vitro, particularly assays relating to the ability of stem cells to give rise to multiple differentiated progeny. Stem cells of interest include pluripotent stem cells (PSCs). The term “pluripotent stem cell” or “PSC” is used herein to mean a stem cell capable of producing all cell types of the organism.

The present disclosure further provides progeny of a genetically modified cell, where the progeny can comprise the same genetic modification as the genetically modified cell from which it was derived. The present disclosure further provides a composition comprising a genetically modified cell. In some embodiments, a genetically modified host cell can generate a genetically modified organism. For example, the genetically modified host cell is a pluripotent stem cell, it can generate a genetically modified organism. Methods of producing genetically modified organisms are known in the art.

Also disclosed herein are methods for genetically modifying diverse bacterial communities (e.g., gut or fecal-derived bacteria). The methods comprise contacting a recipient bacterial community with donor bacteria, the donor bacteria comprising a vector encoding: an engineered Clustered Regularly Interspaced Short Palindromic Repeats (CRISPR)-CRISPR associated (Cas) (CRISPR-Cas) system, wherein the engineered CRISPR-Cas system comprises: (a) at least one Cas protein, and (b) at least one guide RNA (gRNA); an engineered transposon system: and at least one donor nucleic acid to be integrated, wherein the donor nucleic acid is flanked by at least one transposon end sequence and optionally further comprises a cargo nucleic acid. In some embodiments, the vector is conjugative plasmid.

In some embodiments, the vector further encodes a recombinase, or a catalytic domain thereof, and the at least one donor nucleic acid further comprises a recognition site for the recombinase. In some embodiments, the cargo nucleic acid comprises the recognition site for the recombinase.

In some embodiments, the engineered CRISPR-Cas system comprises a pair of guide RNAs (gRNAs), wherein the pair of gRNAs is configured to hybridize to target sites flanking a nucleic acid sequence for deletion. In some embodiments, the nucleic acid sequence for deletion comprises a genomic nucleic acid sequence endogenous to the recipient bacterial community.

The descriptions and embodiments provided above for the components of the CRISPR-Cas system, the transposon system, the recombinase, or catalytic domain thereof, and the donor nucleic acid are applicable to the methods for genetically modifying diverse bacterial communities.

The system and methods may be used in various bacterial hosts, including human pathogens that are medically important, bacterial pests that are key targets within the agricultural industry, human bacteria important for gut or over health, as well as antibiotic resistant versions thereof; e.g., pathogenic Pseudomonas strains, Staphylococcus aureus, Pneumoniae species, Helicobacter pylori, Enterobacteriaceae, Campylobacter spp., Neisseria gonorrhoeae, Enterococcus faecium, Acinetobacter baumannii, E. coli, Klebsiella pneumoniae, etc.

In some embodiments, the microbial cells are Gram-negative bacterial cells, Gram-positive bacterial cells, or a combination thereof.

In some embodiments, the microbial cells are pathogenic bacterial cells. For example, the pathogenic microbial cells may be extended-spectrum beta-lactamase-producing (ESBL) Escherichia coli, Pseudomonas aeruginosa, vancomycin-resistant Enterococcus (VRE), methicillin-resistant Staphylococcus aureus (MRSA), multidrug-resistant (MDR) Acinetobacter baumannii, MDR Enterobacter spp. bacterial cells or a combination thereof.

In some embodiments, the microbial cells are non-pathogenic bacterial cells (e.g., probiotic and/or commensal bacterial cells). In some embodiments, the microbial cells form microbial flora (e.g., natural human microbial flora). Thus, the microbial cells that are members of the phyla Actinobacteria, Bacteroidetes, Proteobacteria, Firmicutes, or others, or a combination thereof, as suitable for use with the disclosed systems and methods.

In some embodiments, the microbial cells are used in industrial or environmental bioprocesses (e.g., bioremediation).

The methods for deleting a nucleic acid sequence, for inactivating a gene of interest, and genetically modifying diverse bacterial communities may be used to inactivate microbial genes. In some embodiments, the gene is an antibiotic resistance gene. For example, the coding sequence of bacterial resistance genes may be disrupted in vivo by insertion of a DNA sequence or deletion of a portion of the bacterial resistance genes, leading to non-selective re-sensitization to drug treatment. In one embodiment, in addition to disruption of resistance genes, when the systems are incorporated on the inserted cargo the present system acts as a replicative transposon and the system can further propagate itself along with the target plasmid.

The present methods may also be used to treat a multi-drug resistance bacterial infection in a subject. Beyond resistance genes, the method may be designed to target any gene or any set of genes, such as virulence or metabolic genes, for clinical and industrial applications in other embodiments. The present methods may be used to target and eliminate virulence genes from the population, to perform in situ gene knockouts, or to stably introduce new genetic elements to the metagenomic pool of a microbiome. For example, the methods may be used to introduce new proteins or enzyme to aid in the digestions of dietary compounds.

The methods may comprise administering to the subject, in vivo, or by transplantation of er vivo treated cells, a therapeutically effective amount of the described system. The components of the described systems, methods, or ex vivo treated cells (e.g., donor bacteria) may be administered with a pharmaceutically acceptable carrier or excipient as a pharmaceutical composition. In some embodiments, the components of the systems and methods may be mixed, individually or in any combination, with a pharmaceutically acceptable carrier to form pharmaceutical compositions, which are also within the scope of the present disclosure.

Administration may be through any suitable mode of administration, including but not limited to: intravenous, intra-arterial, intramuscular, intracardiac, intrathecal, subventricular, epidural, intracerebral, intracerebroventricular, sub-retinal, intravitreal, intraarticular, intraocular, intraperitoneal, intrauterine, intradermal, subcutaneous, transdermal, transmucosal, topical, and inhalation. In some embodiments, the vector(s) is delivered to the tissue of interest by, for example, an intramuscular, intravenous, transdermal, intranasal, oral, mucosal, or other delivery methods. In some embodiments, administering comprises intravenous administration. Such delivery may be either via a single dose, or multiple doses.

In some embodiments, an effective amount of the components of the systems, methods or compositions as described can be administered. As used herein the term “effective amount” may be used interchangeably with the term “therapeutically effective amount” and refers to that quantity that is sufficient to result in a desired activity upon administration to a subject in need thereof. Within the context of the present disclosure, the term “effective amount” refers to that quantity of the components of the system such that successful nucleic acid deletion is achieved.

When utilized as a method of treatment, the effective amount may depend on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. In some embodiments, the effective amount alleviates, relieves, ameliorates, improves, reduces the symptoms, or delays the progression of any disease or disorder in the subject. In some embodiments, the subject is a human.

In the context of the present disclosure insofar as it relates to a disease, the terms “treat,” “treatment,” and the like mean to relieve or alleviate at least one symptom associated with such condition, or to slow or reverse the progression of such condition. Within the meaning of the present disclosure, the term “treat” also denotes to arrest, delay the onset (e.g., the period prior to clinical manifestation of a disease) and/or reduce the risk of developing or worsening a disease. For example, in connection with cancer the term “treat” may mean eliminate or reduce a patient's tumor burden, or prevent, delay, or inhibit metastasis, etc.

The phrase “pharmaceutically acceptable,” as used in connection with compositions and/or cells of the present disclosure, refers to molecular entities and other ingredients of such compositions that are physiologically tolerable and do not typically produce untoward reactions when administered to a subject (e.g., a mammal, a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans. “Acceptable” means that the carrier is compatible with the active ingredient of the composition (e.g., the nucleic acids, vectors, cells, or therapeutic antibodies) and does not negatively affect the subject to which the composition(s) are administered. Any of the pharmaceutical compositions and/or cells to be used in the present methods can comprise pharmaceutically acceptable carriers, excipients, or stabilizers in the form of lyophilized formations or aqueous solutions.

Pharmaceutically acceptable carriers, including buffers, are well known in the art, and may comprise phosphate, citrate, and other organic acids; antioxidants including ascorbic acid and methionine; preservatives; low molecular weight polypeptides: proteins, such as serum albumin, gelatin, or immunoglobulins; amino acids: hydrophobic polymers; monosaccharides; disaccharides; and other carbohydrates: metal complexes: and/or non-ionic surfactants. See, e.g., Remington: The Science and Practice of Pharmacy 20th Ed. (2000) Lippincott Williams and Wilkins, Ed. K. E. Hoover.

Genetic modification may be assessed using techniques that include, for example, Northern blot analysis, in situ hybridization analysis. Western analysis, immunoassays such as enzyme-linked immunosorbent assays, and reverse-transcriptase PCR (RT-PCR). The site of integration or deletion may be determined by Sanger sequencing or next-generation sequencing (NGS).

### 4. KITS

Also within the scope of the present disclosure are kits that include the components of the present system.

The kit may include instructions for use in any of the methods described herein. The instructions can comprise a description of administration of the present system or composition to a subject to achieve the intended effect. The instructions generally include information as to dosage, dosing schedule, and route of administration for the intended treatment. The kit may further comprise a description of selecting a subject suitable for treatment based on identifying whether the subject is in need of the treatment.

The containers may be unit doses, bulk packages (e.g., multi-dose packages) or sub-unit doses. Instructions supplied in the kits of the disclosure are typically written instructions on a label or package insert. The label or package insert indicates that the pharmaceutical compositions are used for treating, delaying the onset, and/or alleviating a disease or disorder in a subject.

The kits provided herein are in suitable packaging. Suitable packaging includes, but is not limited to, vials, bottles, jars, flexible packaging, and the like. Also contemplated are packages for use in combination with a specific device, such as an inhaler, nasal administration device, or an infusion device. A kit may have a sterile access port (for example, the container may be an intravenous solution bag or a vial having a stopper pierceable by a hypodermic injection needle). The container may also have a sterile access port.

Kits optionally may provide additional components such as buffers and interpretive information. Normally, the kit comprises a container and a label or package insert(s) on or associated with the container. In some embodiment, the disclosure provides articles of manufacture comprising contents of the kits described above.

The kit may further comprise a device for holding or administering the present system or composition. The device may include an infusion device, an intravenous solution bag, a hypodermic needle, a vial, and/or a syringe.

The present disclosure also provides for kits for performing the methods or producing the components in vitro. The kit may include the components of the present system. Optional components of the kit include one or more of the following: (1) buffer constituents, (2) control plasmid, (3) sequencing primers.

### 5. EXAMPLES

The following are examples of the present invention and are not to be construed as limiting.

### Methods

Plasmid construction. All V. cholerae INTEGRATE plasmid constructs were generated from pQCascade, pTnsABC, and pDonor using a combination of restriction digestion, ligation. Gibson assembly, and inverted (around-the-horn) PCR. All PCR fragments for cloning were generated using Q5 DNA Polymerase (NEB).

Different plasmid backbone versions of pSPIN were cloned by generating one PCR fragment of the single INTEGRATE transcript and donor and combining with a digested vector backbone in a Gibson assembly reaction. pSPAIN was generated by Gibson assembly; a 0.98-kb mini-Tn was first inserted into a digested empty pBBR1 backbone, followed by double digestion of the cargo within the mini-Tn and insertion of the single INTEGRATE transcript.

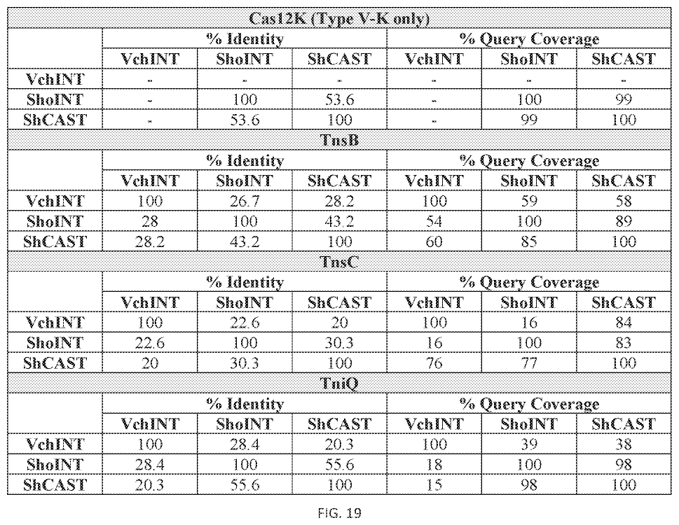

ShoINT system was synthesized by GenScript; Cas12k and the sgRNA were synthesized as two separate cassettes on a pCDFDuet-1 (pCDF) plasmid, TnsA-TnsB-TniQ was synthesized as a native operon on a pCOLADuet-1 (pCOLA) plasmid, and the mini-Tn was synthesized on a pUC19 plasmid. Sho-pEffector and Sho-pSPIN were generated from these plasmids using Gibson assembly. ShCAST system was synthesized by GenScript according to the constructs described previously 40, with pHelper on pUC19 and pDonor on pCDF backbones. Pairwise protein sequence similarities between the VchINT. ShoINT, and ShCAST machinery can be found in FIG. 19.

Each construct containing a spacer was first constructed with a filler sequence containing tandem BsaI recognition sites in place of the spacer for VchINT and ShoINT, and tandem BbsI sites for ShCAST. New spacers were then cloned into the arrays by phosphorylation of oligo pairs with T4 PNK (NEB), hybridization of the oligo pair, and ligation into double BsaI- or BbsI-digested plasmid. Double- and triple-spacer arrays were cloned by combining two or three oligoduplexes with compatible sticky ends into the same ligation reaction. crRNAs for VchINT were designed with 32-nt spacers targeting sites with 5′ CC PAM. sgRNAs for ShoINT and ShCAST were designed with 23-nt spacers targeting sites with 5′ RGTN PAM and 5′ NGTT PAM, respectively. Spacer sequences used for this study are SEQ ID NOs: 132-172. The guide RNA design algorithm (FIG. 16) was not used to generate spacers for this study.

Cloning reactions were transformed into NEB Turbo E. coli, and plasmids were extracted using Qiagen Miniprep columns and confirmed by Sanger sequencing (GENEWIZ). Transformed cells were cultured in liquid LB media or LB agar media, with addition of 100 μg/ml carbenicillin for pUC19 plasmids, 50 μg/ml spectinomycin for pCDF and pSC101*, and 50 μg/ml kanamycin for pCOLA, pSC101 and pBBR1. All plasmid construct sequences are available SEQ ID NOs: 1-113, and a subset are deposited at Addgene.

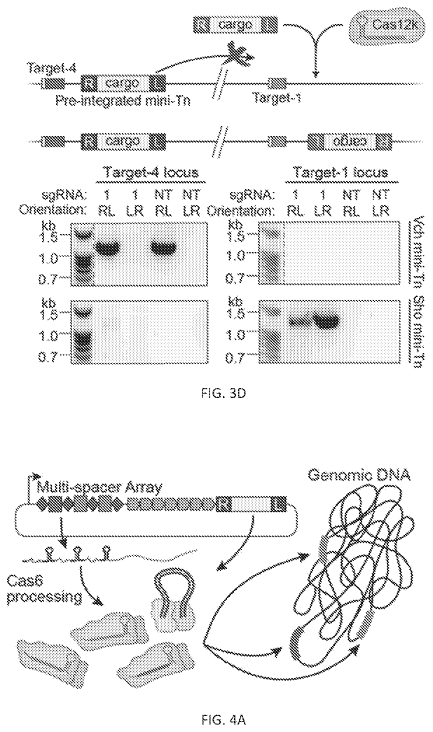

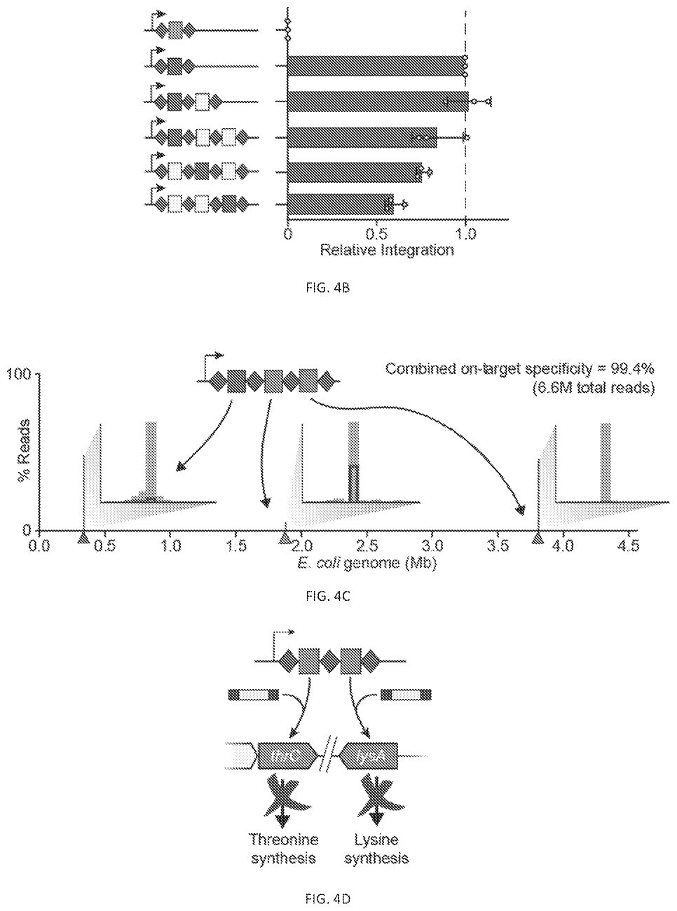

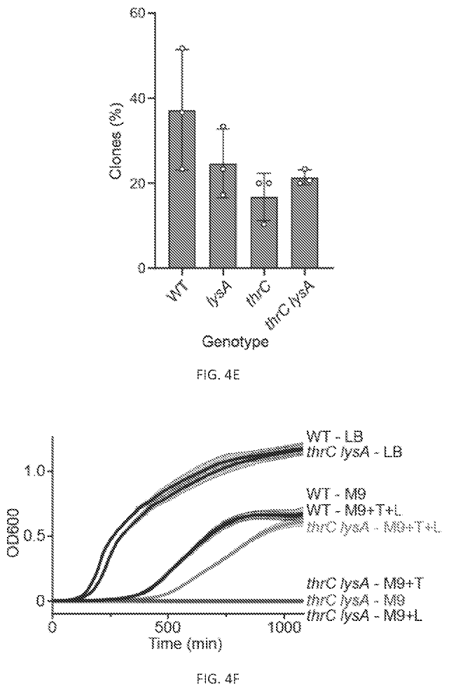

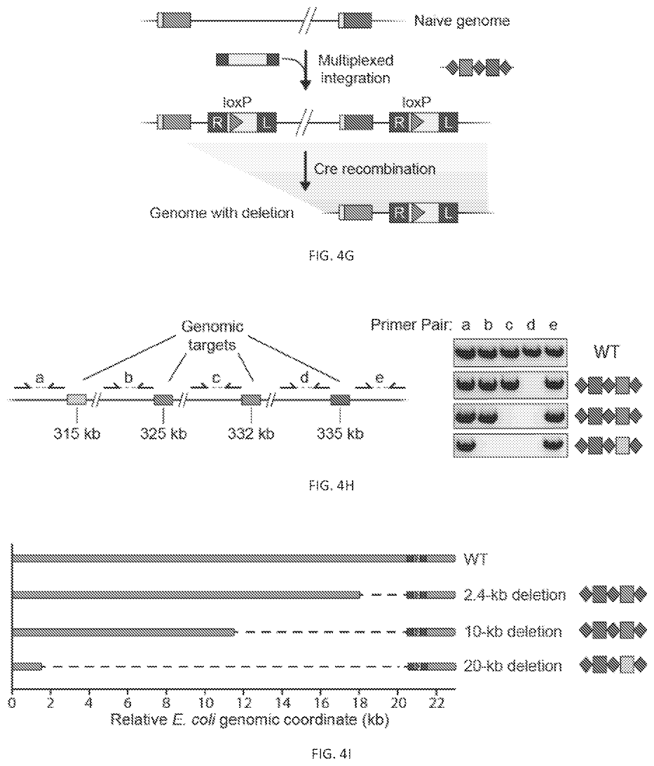

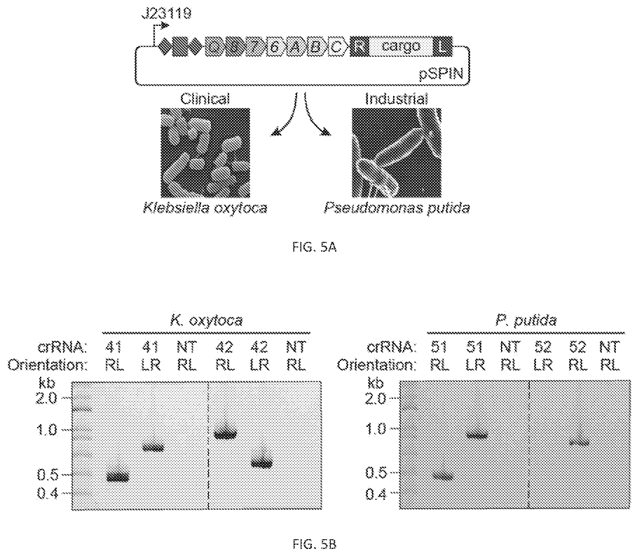

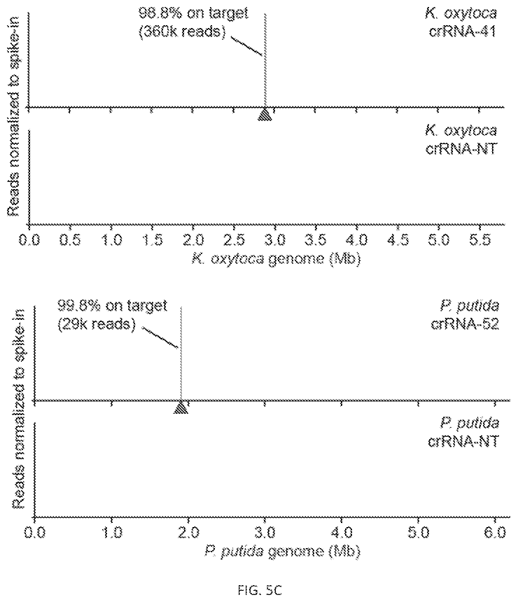

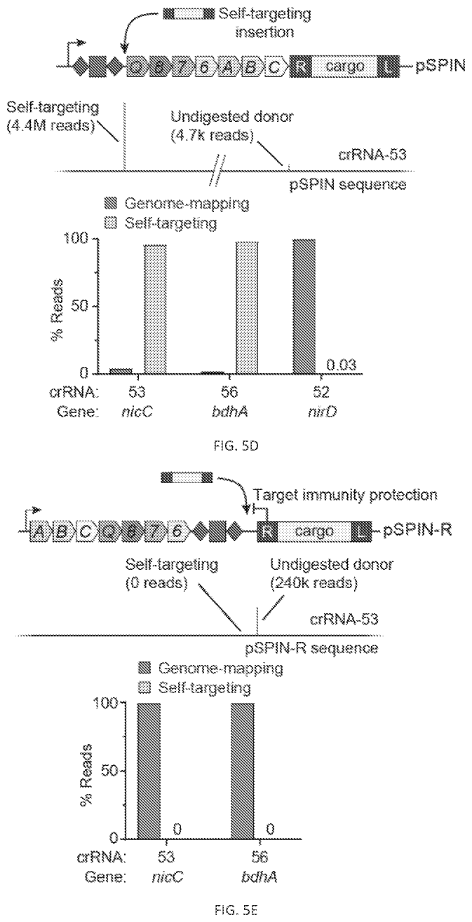

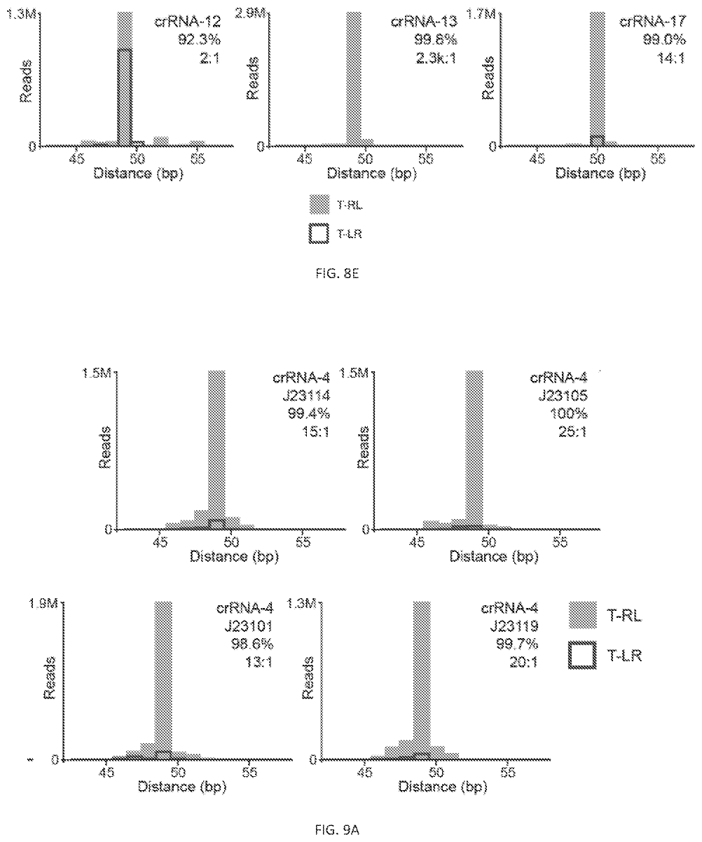

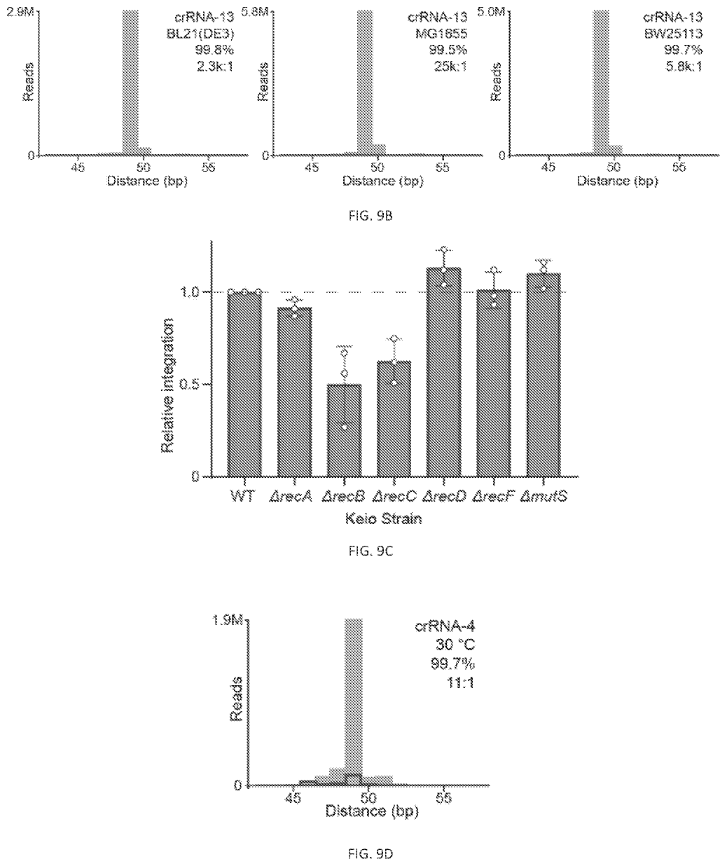

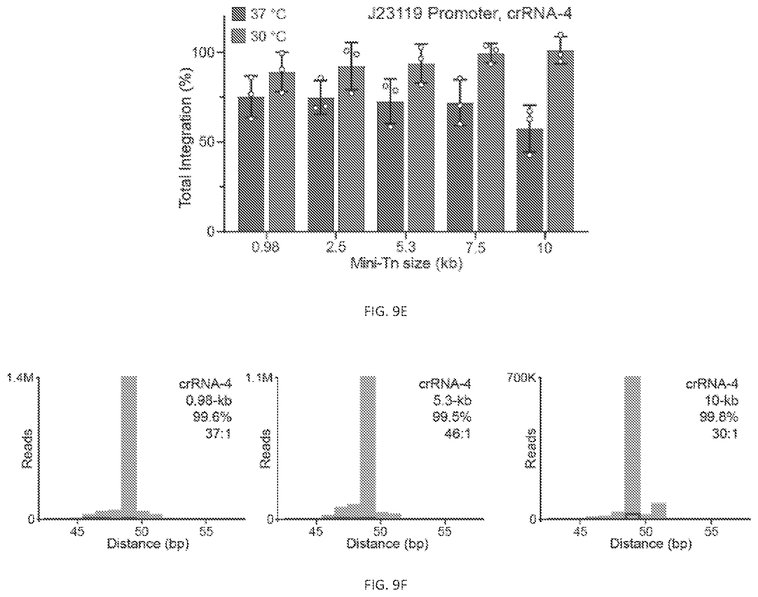

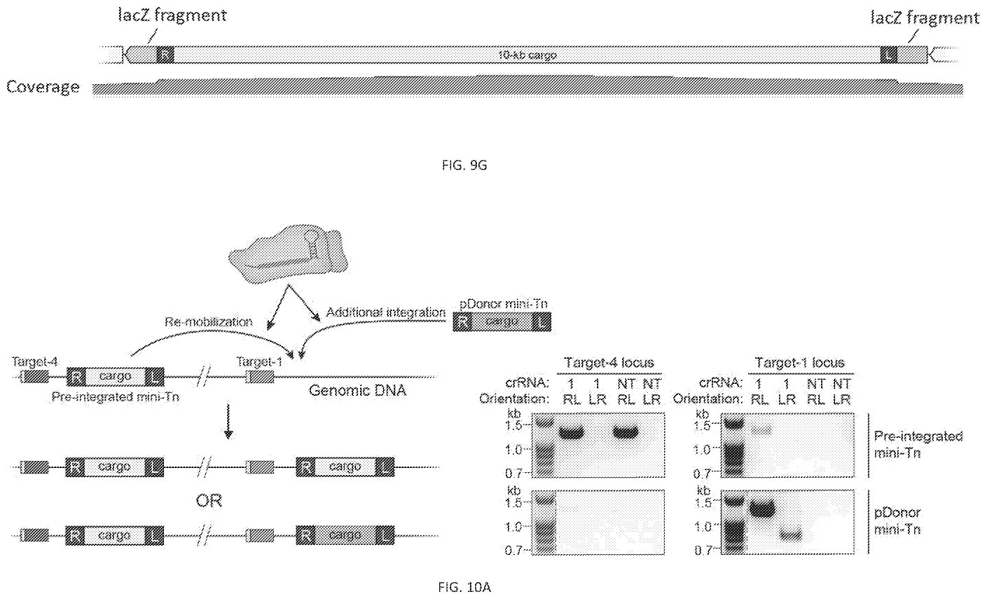

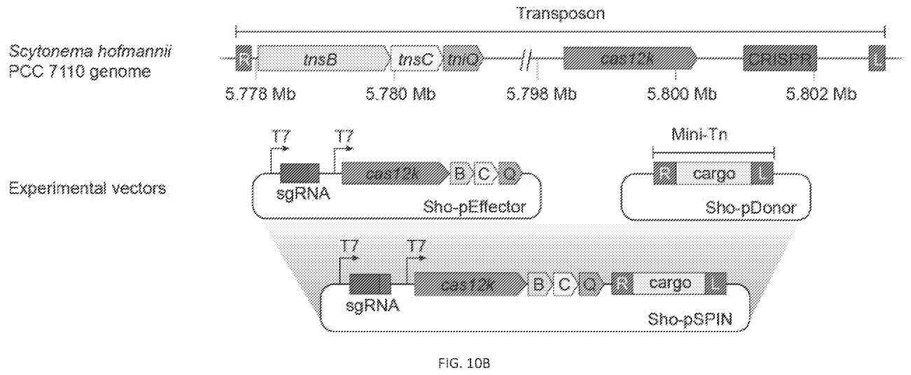

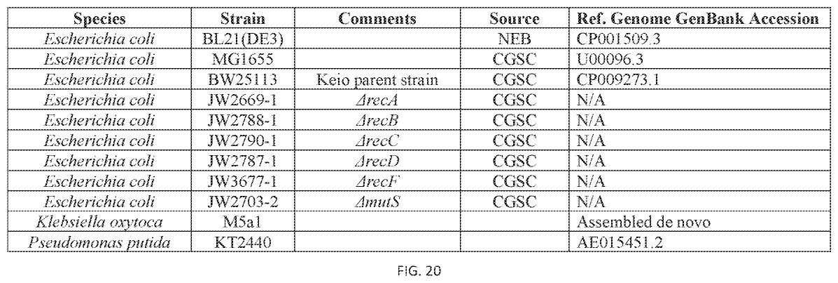

E. coli culturing and general transposition assays. A full list of E. coli strains used for transposition experiments is provided in FIG. 20. All E. coli transformations were performed using homemade chemically competent cells and standard heat shock transformation, followed by recovery in LB at 37° C. and plating on LB-agar media with the appropriate antibiotics at the concentrations described above. Typical transformations efficiencies were >103 CFU/μg of total DNA. All standard transposition assays in E. coli involved incubation at 37° C. for 24 hours after recovery and plating. However, experiments involving incubation at 30° C. or 25° C. were performed for an extended total of 30 hours; due to the reduced growth rate at these temperatures, cells were incubated longer to produce enough cell material for downstream analyses. To control for this extended incubation time, all incubations for FIG. 4C were performed uniformly for 30 hours, including the 37° C. incubations. For similar reasons, incubation for the ΔrecA transposition assays (FIG. 9C) was performed for 30 hours due to significantly slower growth rate of the ΔrecA strain.

For most experiments involving an IPTG-inducible T7 promoter, transformed cells were plated directly on 0.1 mM IPTG LB agar plates for 24 hours after recovery. Exceptions were for the pUC19 pSPIN construct (FIG. 1D), all transposition assays performed for FIG. 1E, and all ShoINT and ShCAST experiments, where transformed cells were first plated on LB agar without induction and incubated for 16 hours, and were then scraped and replated on LB agar with 0.1 mM IPTG and incubated for 24 hours. For these experiments, this replating protocol was generally used since initial transformation efficiencies were low, potentially from significant IPGT induced toxicities affecting transformation; separating transformation and induction steps allowed for enough cells to be generated for lysis and further analyses. To avoid IPTG degradation affecting transposition efficiency, LB agar plates were made fresh with frozen IPTG stocks, and were kept at 4° C. and used within 7 days of preparation. All post-transformation incubations involving active transposition were performed on solid media to avoid competitive growth effects causing enrichment of rare events.

Experiments involving three plasmids—pDonor, pTnsABC and pQCascade or variants—were performed by first transforming pTnsABC and pDonor into chemical competent cells, picking a single colony and growing overnight in liquid LB media with double antibiotic selection, inducing chemical competency using standard methods, and then transforming these cells with the pQCascade plasmid. Experiments involving two plasmids were performed by co-transformation of both plasmids into chemical competent cells simultaneously, although this generally resulted in lower transformation efficiencies and required more input DNA than if the plasmids had been transformed iteratively.

Transposition assays in Klebsiella oxytoca and Pseudomonas putida. A full list of bacterial strains used for transposition experiments is provided in FIG. 20.

For K. oxytoca transformations, cells were grown overnight to saturation, and were diluted 1:100 and grown to OD600 of ˜0.4-0.5. Cells were then placed on ice for 15-30 min and subsequently washed three times with ice-cold 10% glycerol DI water. After the washes, cells were concentrated 100-fold in ice-cold 10% glycerol DI water. 50 μl of cells were electroporated with 50 ng plasmid, using 0.1 cm cuvettes at 1.8 kV. Cells were recovered in 1 ml of LB media for 2 hours at 37° C., and were plated on LB agar with selection at 37° C. for 24 hours.

For P. putida transformations, a previously described protocol was adapted (Aparicio, T., et al., Microb Biotechnol (2019), incorporated herein by reference in its entirety). Briefly, overnight cultures were washed three times with 300 mM sucrose and concentrated 50-fold. Cells were then distributed into 100 μl aliquots and separately electroporated with 100 ng of plasmid using 0.2 cm cuvette at 2.5 kV, and were recovered in 1 ml of LB media for 2 hours at 30° C. Recovered cells were plated on LB agar with selection at 30° C. for 24 hours.

All transposition assays for K. oxytoca and P. putida were performed by transforming a pSPIN construct on a pBBR1 backbone, expressed from a constitutive J23119 promoter. Cells were incubated on LB agar for 24 hours after recovery; colonies were then scraped for gDNA extraction using the Wizard Genomic DNA Purification kit (Promega).

PCR and qPCR analysis of transposition. E. coli cells transformed with INTEGRATE machinery were scraped from LB agar plates and suspended in liquid LB, and the OD600 of the resulting suspensions were taken. From each resuspension, approximately 3.2×10⁸ cells (equivalent to 200 μl of OD600=2.0 of resuspended cells) were taken for lysis. In scenarios where colonies were small and less than this amount of cell resuspension was recovered, the entire resuspension of cells was used for lysis. Cells were pelleted by centrifugation at 4000 g for 2 min, the LB supernatant was poured off and cells were resuspended in 80 μl of DI water, followed by lysis at 95° C. for 10 min. The lysates were cooled to room temperature, pelleted by centrifugation at 4000 g for 2 min, and the supernatant was diluted 20-fold in DI water and used for subsequent analyses. Further dilutions of lysates may be used for analysis, while polymerase inhibition from raw lysates at higher concentrations than the 20-fold dilution, especially for qPCR, have been observed.

PCR reactions for E. coli samples were performed using Q5 Polymerase (NEB) in a 12.5 μl reaction containing 200 μM dNTPs, 0.5 μM of each primer, and 5 μl of diluted lysate supernatant. Primer pairs involved one mini-Tn-specific primer and one genome-specific primer, and each primer pair probes for integration in either T-RL of T-LR orientation. PCR amplicons were generated over 30 PCR cycles, and were resolved by gel electrophoresis on 1-1.5% agarose stained with SYBR Safe (Thermo Scientific). PCR reactions for K. oxytoca and P. putida were done using similar primer design as E. coli, with Q5 Polymerase in a standard 50 μl reaction mixture, and with 20 ng extracted gDNA as input instead of cell lysate.

qPCR reactions were performed on 2 μl of diluted lysates in 10 μl reactions, containing 5 μl SsoAdvanced Universal SYBR Green 2× Supermix (BioRad), 2 μl of 2.5 μM mixed primer pair, and 1 μl H2O. Each lysate sample was analyzed with 3 separate qPCR reactions involving 3 primer pairs: two pairs each involving one mini-Tn-specific primer with one genomic-specific primer probing for either the T-RL or T-LR integration orientation, and one pair with two genome-specific reference primers at the rssA locus. Primer pairs were designed to amplify a product between 100-250 bp, and were confirmed to have amplification efficiencies between 90%-110% using serially diluted lysates. The qPCR primers used in this study are provided in SEQ ID NOs: 172-242. Integration efficiency (%) for each insertion orientation is defined as 100×(2\hat{ }ΔCq), where ΔCq is the Cq(genomic reference pair)−Cq(T-RL pair OR T-LR pair): total integration percentage is the sum of both orientation efficiencies.

Isolation of clonally-integrated E. coli colonies. Due to the potential for colonies becoming polyclonal as integration occurs alongside colony expansion, all clonal isolation steps were preceded by a “bottlenecking” step, where all colonies were scraped, resuspended in LB, and plated at an appropriate dilution to obtain a new set of colonies. Colonies were then picked and resuspended in 100 μl of MQ water, followed by lysis at 95° C. for 10 min. 5 μl of lysate was then used as input template for Q5 PCR as described above. Colonies were identified as clonal using three sets of PCRs per target site per lysate. Briefly, two PCR pairs probed for the presence of either T-RL or T-LR integration, respectively, and a third pair amplified across the genomic region of the expected insertion junction. A colony was considered clonal when only one of the first two primer pairs leads to amplification, and the third pair amplified solely a larger product that corresponds to the genomic region plus the mini-Tn. Where crRNA-4 (targeting lacZ) was used for integration, blue-white screening was used to select for white colonies, which were then confirmed with the above PCR strategy.

Liquid culture time course. pSPIN plasmids with constitutive promoters, which were extracted from NEB Turbo cloning cells, contained contaminating gDNA with targeted integration that was detectable at low levels with both end-point PCR as well as qPCR, especially at early timepoints after transformation with pSPIN. To avoid this artifact for time-course experiments, plasmids were passaged in and extracted from E. coli strain BW25113, which does not have the corresponding genomic site targeted by crRNA-4.

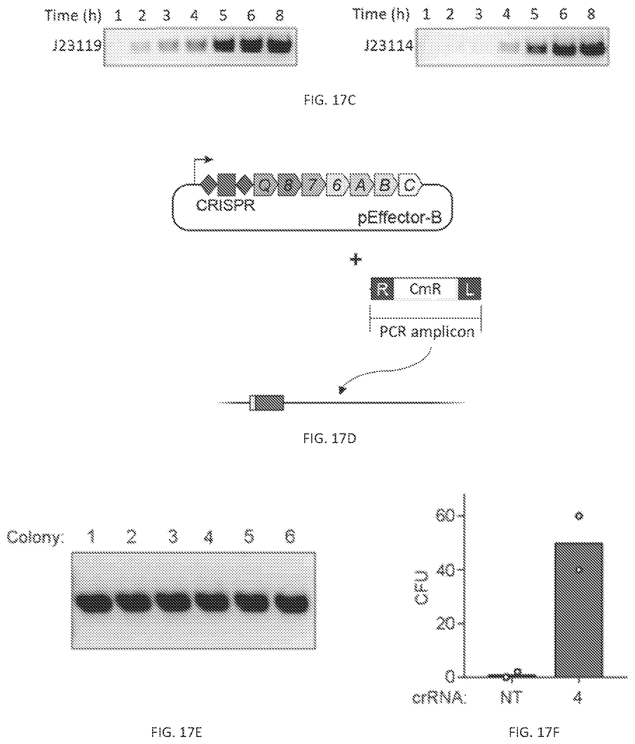

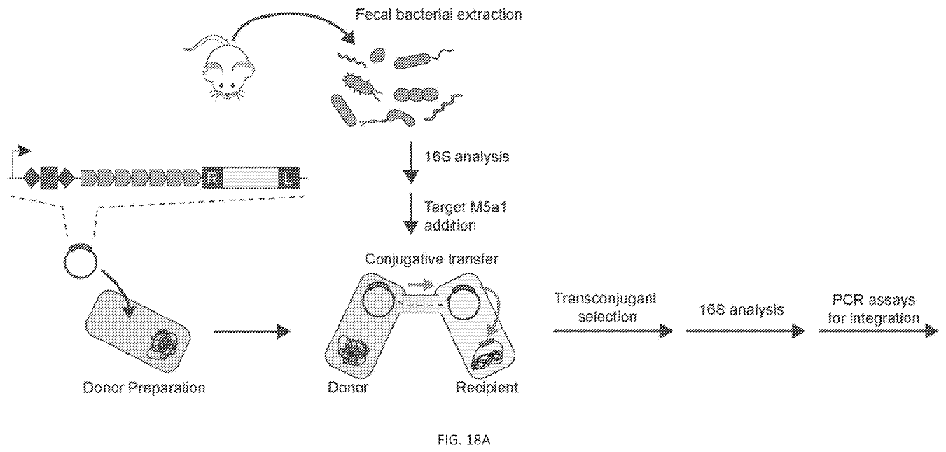

For each sample in the time-course experiment, three separate transformations were performed and were pooled together after a 1-hour recovery at 37° C. The pooled recovery was then split into three equal volumes, each used to inoculate a 25 mL liquid LB outgrowth culture. The cultures were incubated with shaking continuously for 24 h at either 37° C. or 30° C. At each time point indicated in FIG. 17, a 1 mL sample volume was taken from each liquid culture for OD600 measurement (WPA Biowave, 2.0 max reading) and subsequent analysis of integration efficiencies by qPCR. To avoid effects of ongoing integration within these collected samples, samples from liquid cultures were either lysed at 95° C., or frozen at −20° C., within 10 minutes from collection. For earlier time points with dilute cultures, 1 mL samples were pelleted entirely, and each were resuspended in a sufficient volume to achieve a Cq value of 18-20 for the genomic qPCR primer pair. For later time points with significantly turbid cultures, dilution of the sample was performed based on the OD600 measurements, as described in the qPCR section above.

Transposition with linear donor. Linear donors were generated by PCR amplification of a 1104 bp donor sequence containing a full chloramphenicol resistance cassette from a non-replicative plasmid template. A subsequent DpnI digestion and gel extraction step ensured no intact plasmid was present in the linear donor sample. Control transformations of the resulting amplicons were performed into an E. coli pir+ strain that can support replication of the template plasmid to confirm that there was no contaminating plasmid left in the linear DNA sample.

Competent cells carrying a constitutive pEffector plasmid with either a non-targeting crRNA or crRNA-4 were transformed with 500-600 ng of the linear donor using heat-shock transformation as described above. After a 1 h recovery at 37° C., cells were plated directly onto chloramphenicol selection. After a 16 h incubation at 37° C. the resulting colonies were counted. Colonies were then scraped and bottlenecked onto a fresh agar plate with chloramphenicol selection, followed by PCR analysis of colonies as described above.

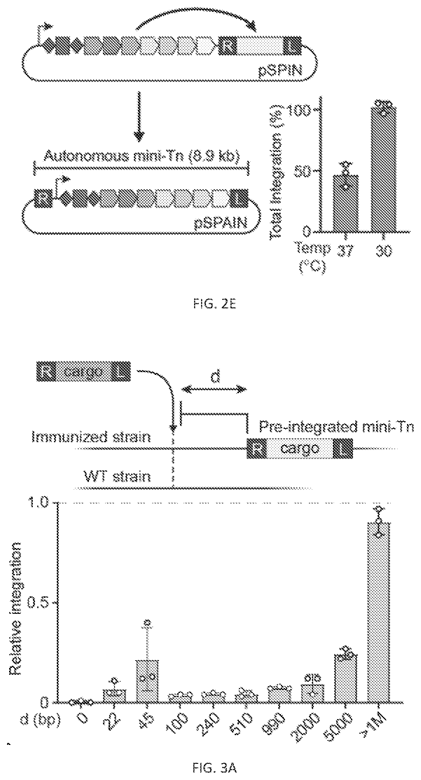

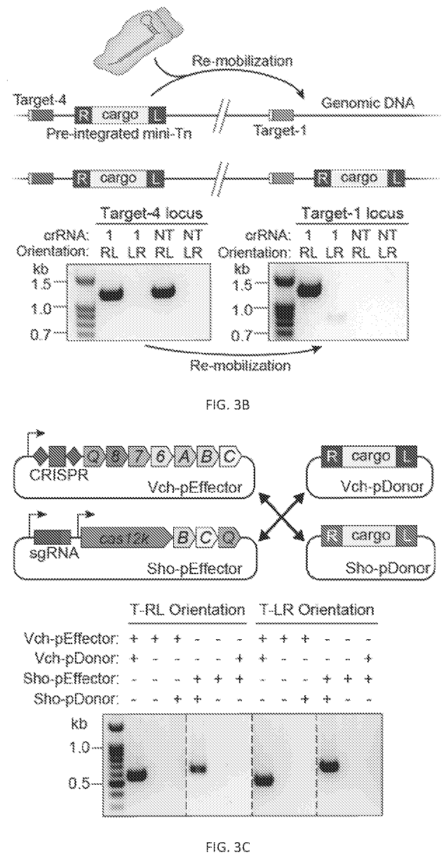

VchINT target immunity experiments. A pSPIN derivative with crRNA-4 (targeting lacZ) on a pSC101* temperature-sensitive backbone was used to insert a 0.98 kb mini-Tn into BL21(DE3) cells at 30° C. for 30 hours. A clonal-insertion strain was isolated as described above, and the pSPIN plasmid was cured by culturing cells at 37° C. overnight in liquid LB media. Resulting cells were made chemically competent, and a separate pDonor containing a different cargo was transformed alongside a pEffector construct with a crRNA targeting a site d (bp) away from the original crRNA-4 target (as indicated in FIG. 3A). qPCR was then performed, where Tn-specific primers were designed to bind in the cargo in order to distinguish it from the original crRNA-4 insertion. For each target site, normalization was done by performing the same transposition and qPCR assay in WT BL21(DE3) cells, and dividing the immunized qPCR efficiency by the WT efficiency. Due to the presence of two identical repeats of the mini-Tn right end and left end (111 bp and 149 bp, respectively) from the original and new insertions, it is possible that the observed target immunity phenotype is affected by low-level recombination between these repetitive sequences, which is not taken into account in the analyses.

Mini-Tn remobilization experiments. BL21(DE3) cells with a clonal crRNA-4 (lacZ) insertion, isolated and cured of INTEGRATE plasmids as described above, was made chemically competent. A pEffector construct with crRNA-1 (targeting downstream of glmS) was transformed into these cells, without a donor plasmid containing a new mini-Tn. Presence of the mini-Tn at both lacZ and glmS was probed for by PCR as described above.

Mini-Tn-competition experiments were set up similarly, where a pEffector construct with crRNA-1 was transformed along with a pDonor which carries the same mini-Tn as the lacZ-insertion, except for a 5-bp mutation at the 3′ end of the R-end. This mutation site was used to design Tn-specific primers to distinguish the genomic-insertion and plasmid-borne mini-Tn at both lacZ and glmS sites.

VchINT/ShoINT orthogonality experiments. For the orthogonality experiments in FIG. 3C, BL21(DE3) cells were co-transformed with a two-plasmid combination of either Vch-pEffector or Sho-pEffector, and either Vch-pDonor or Sho-pDonor. The spacers for both systems were designed to target the same region of the lacZ locus. For PCR analysis of integration activity, due to the two pDonors carrying the same cargo, transposon-specific primers were designed to bind in the R-end or L-end of the mini-Tn.

For data shown in FIG. 3D, Sho-pEffector and Sho-pDonor were co-transformed into BL21(DE3) containing a clonal lacZ-insertion. The Sho-sgRNA was designed with a spacer targeting a similar region near the glmS locus that is targeted by Vch-crRNA-1. PCR analysis was performed as described above.

Amino acid auxotrophy experiments. M9 minimal media was prepared with the following components: 1×M9 salts (Difco), 0.4% glucose, 2 mM MgSO4, and 0.1 mM CaCl2. M9 agar was prepared as above, with the addition of 15 g/l of Dehydrated agar (BD). L-threonine and/or L-lysine was supplemented at 1 mM as indicated.

For individual thrC or lysA targeting experiments, BL21(DE3) cells were transformed with a pSPIN construct with a crRNA targeting either gene. Transformed cells were incubated on LB agar at 37° C. for 24 hours. Bottlenecking and clonal insertions identification by PCR were performed as described above, and cells were then evaluated for ability to grow in M9 minimal media with and without addition of the appropriate amino acid.

For multiplexed targeting of both thrC and lysA, BL21(DE3) cell were transformed with a pSPIN construct expressing a thrC-lysA-targeting double-spacer array. Cells were then incubated and bottlenecked on LB agar as above, and bottlenecked colonies were then stamped onto M9 agar plates supplemented with either no amino acids, only threonine or lysine, or both amino acids, to identify growth phenotype. For data presented in FIG. 4E, this screen was performed on 30 colonies for each of three independent experiments.

OD600 growth curve analysis was performed by first inoculating WT BL21(DE3) or isolated auxotrophic strains from −80° C. glycerol stocks into LB media for overnight growth. 1 ml of each culture was then pelleted at 16000 g and resuspended in 1 ml MQ water, and was inoculated at a 1:1000 dilution into the respective growth media on a 96-well cell culture plate. Growth assay was then performed with a Synergy H1 plate reader shaking at 37° C. for 18 hours, and OD at 600 nm taken every 5 min. Each sample was measured in three technical-replicates in separate wells on the sample plate, and were normalized to blank wells containing media only.

Cre-Lox genomic deletion experiments. BL21(DE3) cells were transformed with a pSPIN construct containing a double-spacer CRISPR array containing crRNA-4 and a second spacer targeting the same strand either 2.4-, 10- or 20-kb away from crRNA-4. The mini-Tn of this construct was previously modified to include a 34-bp recognition sequence for Cre recombinase. Cells were incubated and bottlenecked, and colonies with double-clonal insertions were isolated by a combination of blue-white screening and PCR, as described above. Although the two targets for the 2.4-kb deletion were within each other's range for target immunity effects, the desired clones were still readily isolated. Double-insertion clones were made chemically competent, and were then transformed with a plasmid expressing Cre recombinase from an IPTG-inducible 17 promoter. Cells were incubated at 37° C. for 16 hours and bottlenecked, and colonies having undergone recombination were isolated by PCR. Small colonies and very low transformation efficiencies were observed when transformed cells were plated on 0.1 mM IPTG, while recombined clones were readily able to be isolated without IPTG induction, suggesting that small amounts of Cre resulting from leaky T7 expression were sufficient for recombination. Thus, all Cre-recombinase transformations were performed with no IPTG present.

Tn-seq library preparation and sequencing. Transformations for Tn-seq transposition assays were carried out as described above, using donor plasmids containing a mini-Tn where the 8-nt terminal repeat of the mini-Tn R-end was mutated to contain an MmeI recognition sequence. It was previously shown that a mini-Tn with this mutation is still functionally active, with a ˜50% decrease in total integration efficiency (Klompe, S. E., et al., Nature 571, 219-225 (2019), incorporated herein by reference in its entirety). Transformed cells were incubated on LB agar at 37° C. for 24 hours, except for assays shown in FIGS. 9D and 9F, where cells were incubated at 30° C. for 30 hr. Colonies were then scraped and resuspended in liquid LB media, and 0.5 ml (approximately 2×10⁹ cells) were used for gDNA extraction with the Wizard Genomic DNA Purification kit (Promega), which typically yielded 50 μl of 0.5-1.5 μg/μl gDNA.

NGS libraries were prepared in parallel in PCR tubes, each with 1 μg of gDNA first being digested with 4 U of MmeI (NEB) for 2 hours at 37° C., in a 50 μl reaction containing 50 μM S-adenosyl methionine and 1× CutSmart buffer, followed by heat inactivation at 65° C. for 20 min. MmeI digestion results in the generation of 2-nucleotide 3′-overhangs. Reactions were cleaned up with 1.4× Mag-Bind TotalPure NGS magnetic beads (Omega) according to the manufacturer's instructions, and elutions were done using 30 μl of 10 mM Tris-Cl, pH 7.0. Double-stranded i5 universal adaptors containing a 3′-terminal NN overhang were ligated to the MmeI-digested gDNA in a 20 μl ligation reaction consisting of 16.86 μl of MmeI-digested gDNA, 5 nM adaptor, 400 U T4 DNA ligase (NEB), and 1×T4 DNA ligase buffer. Reactions were left at room temperature for 30 min, and were then cleaned with magnetic beads. Since the donor plasmid (either pDonor, pSPIN or pSPIN-R) contains a copy of the mini-Tn that can also be digested with MmeI and ligated with i5 adaptor, a restriction enzyme recognition site (HindIII for pDonor, or Bsu36I for pSPIN and pSPIN-R) was included in the 17-bp space between the 5′ end of the mini-Tn and the MmeI digestion site. This allowed us to reduce contamination of donor sequences within the NGS libraries, by digesting the entirety of the adaptor-ligated gDNA elution with 20 Units of HindIII or Bsu361 in a 34.4 μl reaction for 2 hours at 37° C., before a heat inactivation step at 65° C. for 20 min. DNA clean-up using magnetic beads was then performed.

Eluted DNA was then amplified in a PCR-1 step, where adaptor-ligated transposons were enriched using a universal i5-adaptor primer and a transposon-specific primer with a 5′ overhang containing a universal i7 adaptor. In a 25 μl PCR-1 reaction. 16.7 μl of HindIII/Bsu36I-digested gDNA was mixed with 200 μM dNTPs, 0.5 μM primers, 1×Q5 reaction buffer, and 0.5 U Q5 DNA Polymerase (NEB). Amplification proceeded for 25 cycles at an annealing temperature of 66° C. 20-fold dilutions of the reaction products were used as template for a second 20 μl PCR reaction (PCR-2) with indexed p5/p7 Illumina primers. The PCR-2 reaction was subjected to 10 additional amplification cycles with an annealing temperature of 65° C., after which analytical gel electrophoresis was performed to verify amplification for each library. Barcoded reactions were pooled and resolved by 2.5% agarose gel electrophoresis, followed by isolation of DNA using Gel Extraction Kit (Qiagen), and NGS libraries were quantified by qPCR using the NEBNext Library Quant Kit (NEB). Illumina sequencing was performed with a NextSeq mid-output kit with 150-cycle single-end reads and automated adaptor trimming and demultiplexing (Illumina). The plasmid contains a full-size MmeI-mini-Tn, where there is no Bsu36I restriction site in the 17-bp fingerprint space—thus this fingerprint survives the Bsu36I donor digestion step for pSPIN libraries, and provides a constant “contamination” into the library to control for sequencing depth.

For pSPIN libraries involving a spike-in, 10 μl of a 0.02 ng/p spike-in plasmid was added to each 1 μg DNA sample prior to MmeI digestion, and library preparation proceeded as described above. The plasmid contains a full-size MmeI-mini-Tn, where there is no Bsu36I restriction site in the 17-bp fingerprint space—thus this fingerprint survives the Bsu36I donor digestion step for pSPIN libraries, and provides a constant “contamination” into the library to control for sequencing depth.

Random fragmentation library prep and sequencing. BL21(DE3) cells were transformed with Vch-pSPIN or Sho-pSPIN, or were co-transformed with pHelper and pDonor for ShCAST. Transformation, incubation and gDNA extraction with the Wizard Genomic DNA Purification kit (Promega) were performed as described previously.

Following the NEBNext® dsDNA Fragmentase protocol, about 2.5 μg of gDNA was fragmented for 14 min. The fragmentation reactions were purified using 1.4× Mag-Bind® Total Pure NGS (Omega) beads with an elution step in 30 μl 1×TE. Approximately 1 μg of the fragmented DNA was used for end preparation, adapter ligation and USER cleavage, according to the NEBNext Ultra II DNA Library Prep Kit for Illumina protocol. The reactions were purified using 1.2× Mag-Bind® Total Pure NGS (Omega) beads with an elution step in 30 μl MQ water.

To reduce the number of fragments deriving from the mini-Tn on the donor plasmid, the samples were digested with restriction enzymes (VchINT-KpnI/Bsu36I, ShoINT-PstI/HindIII, ShCAST-NcoI/AvrII) overnight at 37° C. The reactions were then purified using 1.2× Mag-Bind® Total Pure NGS (Omega) beads with an elution step in 30 μl MQ water.

PCR-1 reactions were performed using Q5 Polymerase (NEB) in a 20 μl reaction containing 200 μM dNTPs, 0.5 μM of each primer, and 30 ng of input DNA. A transposon-specific primer carrying an i5 adapter, and an i7-specific primer specifically amplified transposon containing fragments over 20 PCR cycles. A second PCR reaction (PCR-2) was used to add specific Illumina index sequences to the i5 and i7 adapters over 10 PCR cycles in a 25 μl reaction with 1.25 μl from PCR-1 as the input DNA.

Samples were purified using the Qiagen PCR Clean-up Kit, and their DNA concentrations were measured using a DeNovix spectrometer. The amount of DNA was normalized and samples were combined. The pooled libraries were then quantified using the NEBNext® Library Quant Kit for Illumina, and Illumina sequencing was performed as described above.

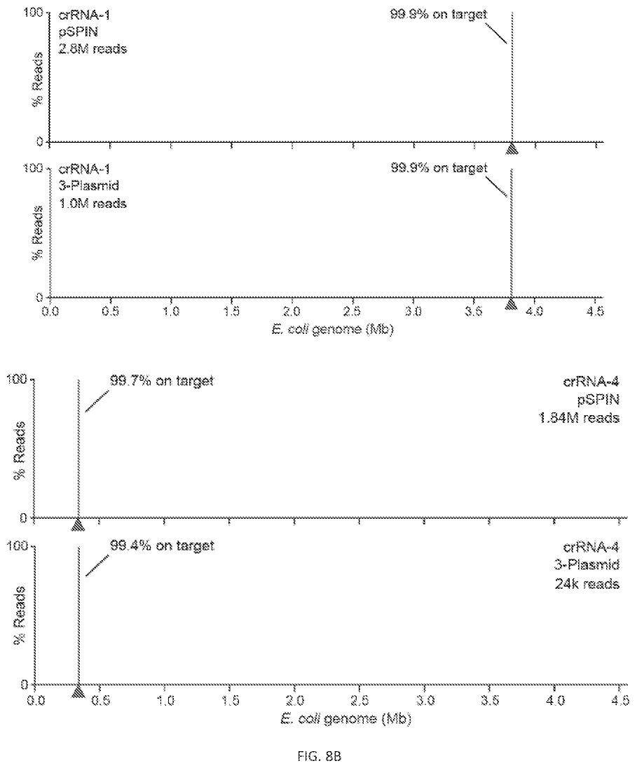

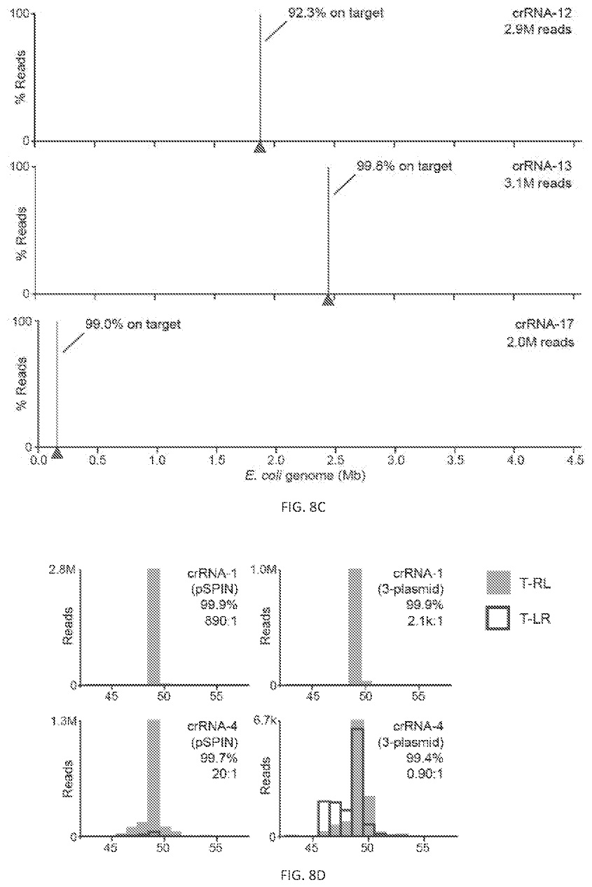

Analysis of NGS data. All analyses of Tn-seq and random fragmentation sequencing data were performed using a custom Python pipeline. Demultiplexed raw reads were filtered to remove reads where less than half of the bases passed a Phred quality score of 20 (Q20—corresponding to >1% base miscalling). Reads that contained the 15-bp 5′ terminal sequence of the mini-Tn R-end (allowing up to one mismatch) were then selected, and the 17-bp sequence directly upstream of this R-end sequence was extracted. This 17-bp “fingerprint” sequence corresponds to the distance from the R-end to the MmeI digestion site, and contains the sequence context in which the mini-Tn is found (FIG. 8A). Reads without sufficient length to extract a 17-bp fingerprint were removed from analysis. For each random fragmentation sample, since the two transposon ends were amplified and sequenced as two separate libraries, extraction of fingerprints from reads were performed separately for the R and L transposon ends.

Fingerprint sequences were aligned to reference genomes of the corresponding species and strain, depending on each specific library. The full list of strains, species, and corresponding reference genome accession identifiers is provided in FIG. 20: reference genomes for E. coli and P. putida were obtained from published NCBI genomes, whereas the K. oxytoca parent strain was sequenced and assembled de novo using whole-genome SMRT sequencing to obtain the reference genome (see below for SMRT sequencing method). Alignment to reference was performed using the bowtie2 alignment library—perfect mapping was used for alignment, and only reads that aligned exactly once to the reference genome were used for downstream analyses. Fingerprints that did not map to the reference genome were screened for sequences corresponding to undigested donor contamination, or for fingerprints mapping downstream of the CRISPR array on the donor plasmid, which correspond to self-targeting events (FIGS. 5D and 5E). For cases where a spike-in plasmid was used, the number of fingerprints containing the spike-in sequence was also determined.

Bowtie2 alignment outputs were used to generate genome-wide integration distributions, the number of reads corresponding to integration events at each position across the reference genome was plotted. For visualization purposes, these positions were grouped into 456 separate 10-kb bins, and peaks were plotted as a percentage of total reads. In cases where a spike-in was used, peaks were further normalized by the number of spike-in fingerprints detected, and the plot each non-targeting control was plotted to the same y-axis scale as its corresponding targeting sample. This analysis was performed similarly for each random fragmentation library by combining R-end and L-end fingerprints prior to alignment and plotting.

Integration-site distance distribution plots were generated from bowtie2 alignments by plotting number of reads against the distance between the 3′ end of the protospacer and the site of insertion corresponding to the reads, at single-bp resolution. The on-target % was calculated as the percentage of reads corresponding to integration events within a 100-bp window centered at the integration site with the largest number of reads. The orientation bias of integration which was define as the ratio of number of reads corresponding to T-RL insertions to those corresponding to T-LR insertions. For random fragmentation libraries, alignments for this analysis were performed separately for R-end and L-end fingerprints, and the results were combined to generate the plot.

Tn-seq sequencing is susceptible to potential biases arising from differences in MmeI digestion efficiency at each site, and in ligation efficiencies of 3′-terminal NN overhang adaptors, which were not taken into account by downstream analyses.

PacBio SMRT sequencing and analysis. gDNA samples for library preparation were extracted from overnight LB cultures using the Wizard Genomic DNA Purification kit (Promega) as described above. Multiplexed microbial whole genome SMRTbell libraries were prepared as recommended by the manufacturer (Pacific Biosciences). Briefly, two micrograms of high molecular weight genomic DNA from each sample (n=12 per pool) was sheared using a gO-tube to ˜10 kb (Covaris). These sheared gDNA samples were then used as input for SMRTbell preparation using the Template Preparation Kit 1.0, where each sample was treated with a DNA Damage Repair and End Repair mix, in order to repair nicked DNA and repair blunt ends. Barcoded SMRTbell adapters were ligated onto each sample in order to complete SMRTbell library construction, and then these libraries were pooled equimolarly, with a final multiplex of 12 samples per pool. The pooled libraries were then treated with exonuclease III and VII to remove any unligated gDNA, and cleaned with 0.45× AMPure PB beads to remove small fragments and excess reagents (Pacific Biosciences). The completed 12-plex pool was annealed to sequencing primer V3 and bound to sequencing polymerase 2.0 before being sequenced using one SMRTcell 8M on the Sequel 2 system with a 20-hour movie.

After data collection, the raw sequencing reads were demultiplexed according to their corresponding barcodes using the Demultiplex Barcodes tool found within the SMRTLink analysis suite, version 8.0. Demultiplexed subreads were downsampled 10-fold by random downsampling, and assembled de novo using the Hierarchical Genome Assembly Process (HGAP) tool, version 4.0 using the following parameters: Aggressive mode=off, Downsampling factor=0, Minimum mapped length=50 bp, Seed coverage=30, Consensus algorithm=best, Seed length cutoff=−1, Minimum Mapped Concordance=70%.

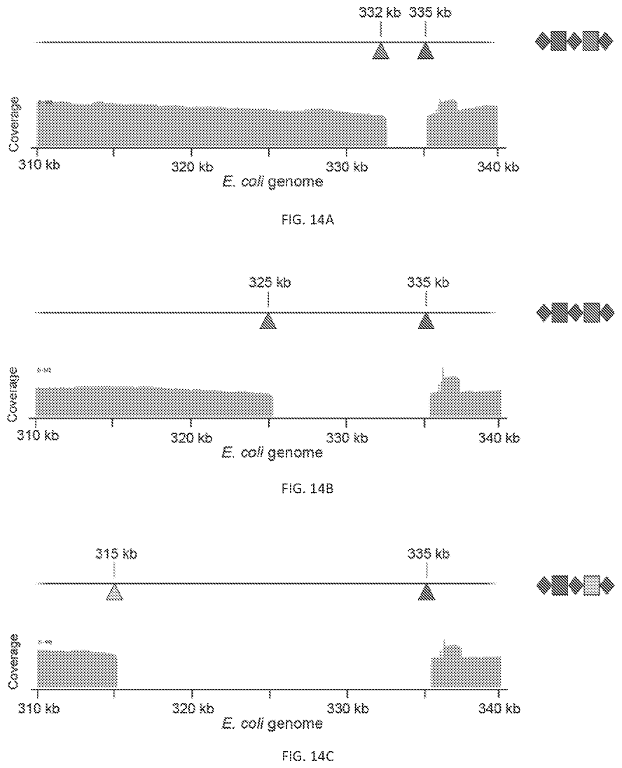

Subread mapping and structural variant analysis were performed using the PB-SV tool within SMRTLink 8.0, using the BL21(DE3) genome (Accession CP001509.3) as reference, with the following parameters: Minimum SV length=20 bp, Minimum reads supporting variant for any one sample=2, Minimum mapped length=50 bp, Minimum length of copy number variant=1000 bp, Minimum reads supporting variant (total over all samples)=2, Minimum % of reads supporting variant for any one sample=20%, Minimum mapped concordance=70%. VCF outputs was used to generate SV analysis results, and BAM alignments were visualized with IGV to generate genome-deletion coverage plots (FIG. 14). No evidence of co-integrate products was found for Vch INTEGRATE, consistent with transposition proceeding through a cut-and-paste pathway dependent on both TnsA and TnsB.

For the coverage plot of the 10-kb insertion (FIG. 9G), circular consensus sequence (CCS) reads were generated with SMRTLink 8.0, and were then filtered using a custom Python script to obtain only reads containing 20 bp of the R-end and/or the L-end of the mini-Tn, where the 20-bp regions directly adjacent to these R/L-end sequences do not map to pDonor. These filtered reads were then aligned to an artificial reference genome, where the entire 10-kb mini-Tn was inserted 49 bp downstream of the crRNA-4 target sequence of the CP001509.3 reference genome, using Geneious Prime at medium sensitivity with no fine-tuning.

Isolation of live mouse gut bacteria. Conventionally raised B6 and BALB/C female mice (Taconic Biosciences Laboratories) were the source of the two different mammalian gut complex communities used in this study. Fresh fecal pellets were collected from mice, and live gut bacteria were isolated by mechanical homogenization. Briefly, 250 μl of PBS was added to previously weighed pellets in a microcentrifuge tube. Pellets were thoroughly mechanically disrupted with a motorized pellet pestle, and then 750 μl of PBS was added. The disrupted pellets in PBS were then subjected to four iterations of vortex mixing for 15 s at medium speed, centrifugation at 1,000 r.p.m. for 30 s at room temperature, recovery of 750 μl of supernatant in a new tube, and replacement of that volume of PBS before the next iteration. The resulting 3 ml of isolated cells were pelleted by centrifugation at 4,000 g for 5 min at room temperature, the supernatant was discarded, and cells were resuspended in 0.5-1.0 ml of PBS. All gut bacteria isolations were performed in an anaerobic chamber (Coy Labs).

Ex vivo conjugation using INTEGRATE to target specific strains in natural complex communities. Before conjugation, donor strains harboring conjugative pSPIN vectors were grown from a single colony in 5 ml of LB-Lennox media (BD) supplemented with 50 μg/ml kanamycin and 50 μM DAP at 37° C. overnight (˜10 h). The recipient community was isolated anaerobically from fresh mouse feces as described above, immediately before conjugation. Donor cells were washed three times in PBS and quantified by OD600, whereas fecal bacteria were quantified by flow cytometry using SYTO9 staining. 108 or 107 donor cells (E. coli strain EcGT2 containing pSPIN) and 108 target cells (K. oxytoca strain M5a1) were mixed with 109 fecal bacteria cells, pelleted by centrifugation at 4,000 g, and resuspended in 10-20 μl of PBS. The mixes were spotted on MGAM+2% agar plates supplemented with 50 μM DAP and incubated at 37° C. anaerobically for 24 h. After conjugation, cells were scraped from the plate into 1 ml of PBS and plated on LB-Lennox agar and LB-Lennox 2% agar supplemented with 50 μg/ml kanamycin at different dilutions.

Metagenomic 16S sequencing. Genomic DNA from fecal bacterial extraction was isolated using mechanical lysis with 0.1 mm Zirconia beads (Biospec) and subsequently purified with SPRI beads (AMPure). PCR amplification of the 16S rRNA V4 region and multiplexed barcoding of samples were done in accordance with previous protocols. The V4 region of the 16S rRNA gene was amplified with customized primers according to the method described by Kozich et al. (Appl Environ Microbial 79, 5112-5120 (2013), incorporated herein by reference in its entirety), with the following modifications: (i) alteration of 16S primers to match updated EMP 505f and 806rB primers, and (ii) use of NexteraXT indices such that each index pair was separated by a Hamming distance of >2, so that Illumina low-plex pooling guidelines could be used. Sequencing was done with the Illumina MiSeq system (300V2 kit) immediately before the experiment (TO) and after 24 h (T24).

Analysis of 16S next-generation sequencing data. The composition of the communities for each sample was determined from 16S sequencing data via DADA2 pipeline to generate the amplicon sequence variance (ASV) tables and calculate relative abundances. Phyloseq and Silva database were used to assign the taxonomy. In the MiSeq run, two blank controls with sterile water as input material were included to check for contaminants in the reagents, and to filter out contaminant ASVs if present. Reads mapping to nonbacterial DNA (e.g., mitochondria, plastids, or other eukaryotic DNA) were also excluded from the analysis. Only ASVs with more than 15000 reads and present in more than 1% of the samples were considered in the downstream analysis.

Quantification of site-specific transposition efficiency in bacterial communities. Different dilutions from the community conjugations were plated on LB with kanamycin selection (50 μg/mL) for pSPIN. Between 40 to 66 colonies were picked each single experiment (˜15 to 20 colonies per replicate in order to capture at least 5% efficiency), and transposon-genome junction PCRs and 16S PCRs were run for each single colony. Junction PCRs were run on 1% agarose gel to confirm the integration, and 16S Sanger sequencing confirmed that each colony was K. oxytoca.

### Example 1

### An Optimized, Single-Plasmid System for High-Efficiency RNA-Guided DNA Integration

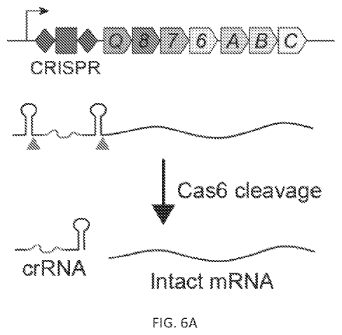

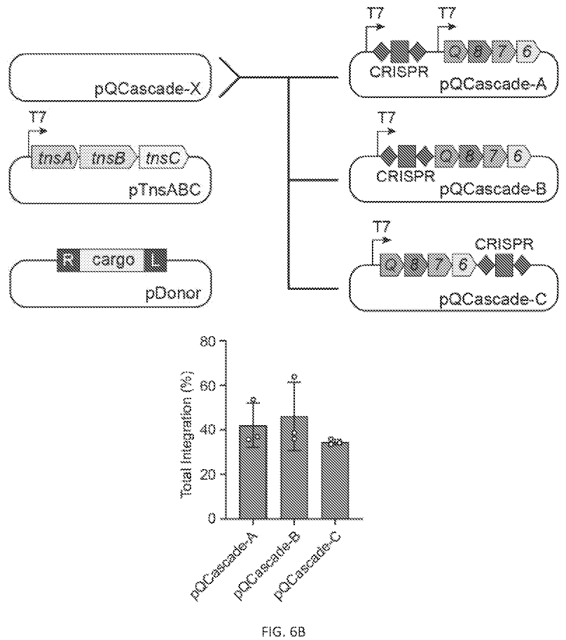

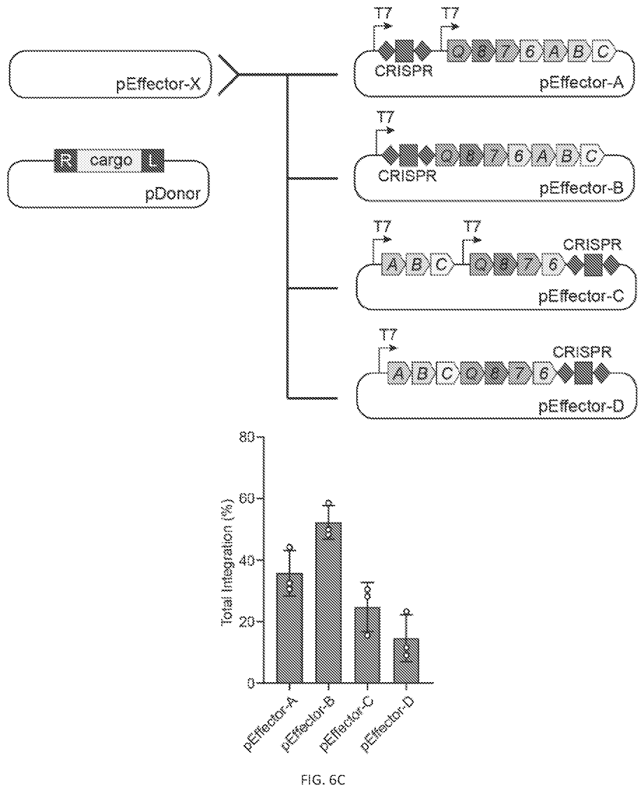

A three-plasmid expression system was previously employed to reconstitute RNA-guided DNA integration in E. coli, whereby pQCascade and pTnsABC encoded the necessary protein-RNA components, and pDonor contained the mini-transposon (mini-Tn, aka donor DNA) (FIG. 1C). To streamline the strategy and eliminate both antibiotic burden and the need for multiple transformation events, number of independent promoters and plasmids was serially reduced the and ultimately arrived at a single-plasmid INTEGRATE construct (pSPIN), in which one promoter drives expression of the crRNA and polycistronic mRNA, directly upstream of the mini-Tn (FIGS. 1C and 6). This design allows modular substitution of the promoter and/or genetic cargo for user-specific applications, and straightforward subcloning into distinct vector backbones.

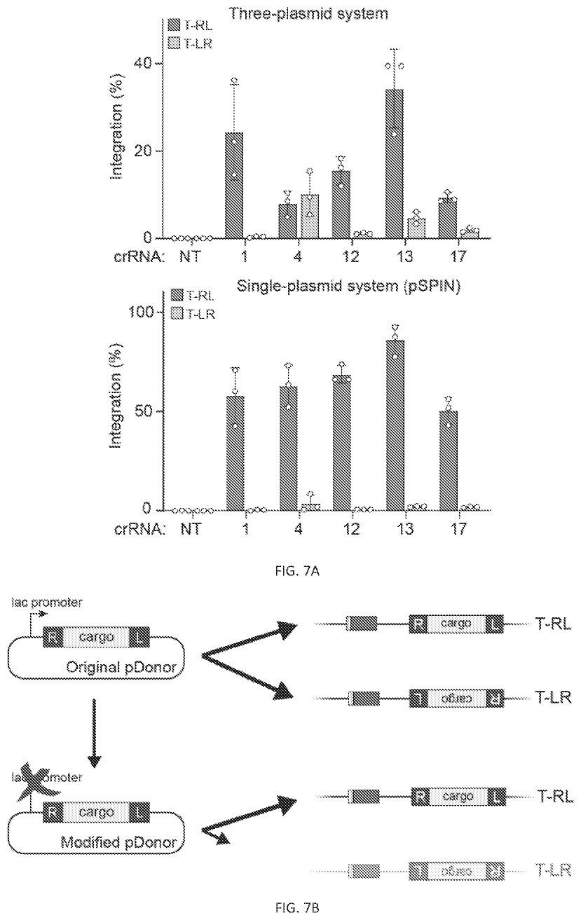

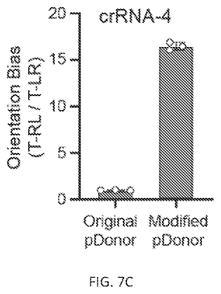

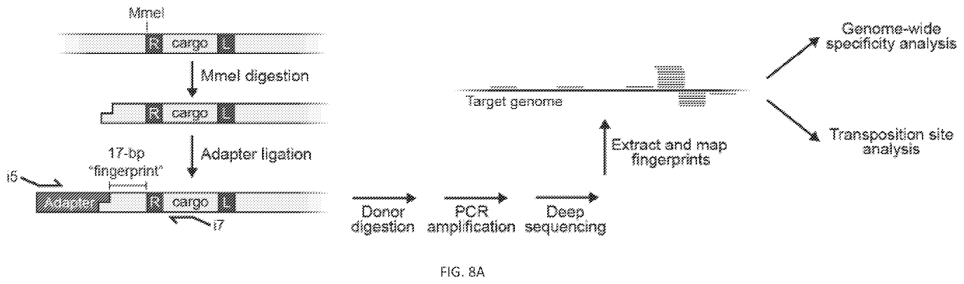

After identifying a functionally optimal arrangement of the CRISPR array and operons (FIG. 6), E. coli BL21(DE3) was transformed with four pSPIN derivatives encoding a lacZ-specific crRNA on distinct vector backbones, and the efficiency of RNA-guided transposition was monitored by quantitative PCR (qPCR). Surprisingly, the streamlined plasmids exhibited enhanced integration activity, with efficiency exceeding 90% using the pBBR1 vector backbone (FIG. 1D), and showed substantially stronger bias for insertion events in which the transposon right end was proximal to the target site (T-RL), as compared to the original three-plasmid expression system (FIG. 7). To determine whether increased efficiency would translate across multiple targets, integration at five target sites, the pSPIN vector was assessed and consistently 2-5× more efficient (FIG. 1E). The single-plasmid INTEGRATE system maintained high-fidelity activity, and an absence of insertion events with a non-targeting crRNA, as reported by genome-wide transposon-insertion sequencing (Tn-seq: FIGS. 1F and 8). This high degree of specificity was further verified by isolating clones and confirming the unique presence of a single insertion by whole-genome, single-molecule real-time (SMRT) sequencing and structural variant analysis.

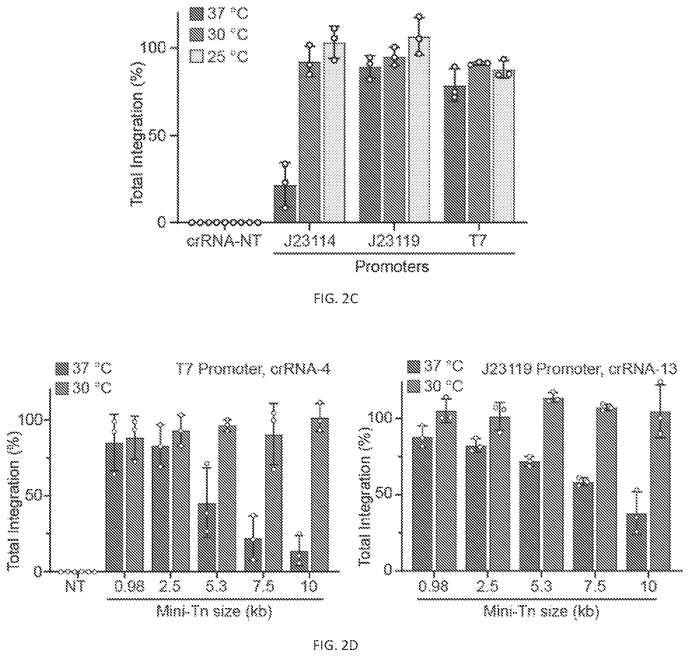

Using a panel of constitutive promoters of varying expression strength, higher expression drove higher rates of integration, without any deleterious effect on genome-wide specificity (FIGS. 2A-2B and 9A). Efficient integration was also driven by a natural broad-host promoter recently adopted for metagenomic microbiome engineering (FIG. 2A), and the use of constitutive promoters allowed demonstration of high-accuracy integration in additional E. coli strains, including MG1655 and BW25113, without any requirement for host recombination factors (FIGS. 9B-9C). Interestingly, that RNA-guided DNA integration readily proceeded when cells were grown at room temperature, and reached ˜100% efficiency (without selection for the integration event) while maintaining 99.7% on-target specificity, even for the low-strength J23114 promoter (FIGS. 2C and 9D).

The kinetics of transposition in liquid culture experiments were also followed. For both strong and weak promoters, the integration efficiency plateaued as the cells approached stationary phase at 37° C., suggestive that rapid growth of the bacterial population at higher temperatures can limited transposition (FIG. 17A). This effect was most apparent for the low-strength J23114 promoter, where the slower onset of exponential growth at 30° C. allowed more time for integration to reach its maximum efficiency of ˜90%. In addition, simple dilution of a culture grown at 37° C. into fresh media also boosted integration efficiencies (FIG. 17B). It was also found that integration products could be detected within 2 hours after transformation (FIG. 17C), suggestive that the system can be deployed without conventional replicating plasmids. When the donor DNA encoding chloramphenicol resistance was delivered into cells in the form of a linear PCR product, drug-resistance clones that uniformly contained the on-target insertion were readily isolated (FIG. 17D).

It was previously found that while the V. cholerae machinery integrated a ˜1-kb cargo with optimal efficiency, larger cargos were poorly mobilized. Remarkably, when protein-RNA components were expressed from a single effector plasmid (pEffector-B, FIG. 6C) and cultured cells at 30° C., mini-transposons spanning 1-10 kb with ˜100% efficiency, were integrated with no observable size-dependent effects (FIG. 2D) and without the need for marker selection. The same pattern was observed across multiple target sites and promoters, and the specificity of 10-kb insertions was verified by Tn-seq and SMRT sequencing (FIGS. 2D and 9e-9G). To further leverage the large-cargo capability, a single-plasmid autonomous INTEGRATE system (pSPAIN) was generated in which the protein-RNA coding genes were cloned within the mini-Tn itself, and this construct also directed targeted integration at ˜100% efficiency (FIG. 2E). Autonomous INTEGRATE systems, by virtue of mobilizing themselves according to the user-defined CRISPR array content, are capable of programmed self-propagation in mixed community environments.

### Example 2

### Development of Orthogonal Integrases for Iterative DNA Insertions

A derivative of pSPIN was cloned using a temperature-sensitive plasmid backbone, a clonal strain containing a lacZ-specific insertion (target-4) was isolated, and the plasmid was cured. Next, the machinery to generate a proximal insertion at variable distances was re-introduced upstream of target-4, but using a mini-Tn whose distinct cargo could be selectively tracked by qPCR (FIG. 3A). Previous studies have demonstrated that Tn7 and Tn7-like transposons exhibit target immunity, whereby integration is prevented at target sites already containing another transposon copy. Integration across a panel of crRNAs for strains with and without a pre-existing mini-Tn was compared and the V. cholerae transposon also exhibited target immunity, with ˜20% relative efficiency at target sites ˜5-kb away (FIG. 3A). This effect was ablated when a glmS-proximal site (target-1) that was >1 Mbp from the pre-existing insertion was targeted.

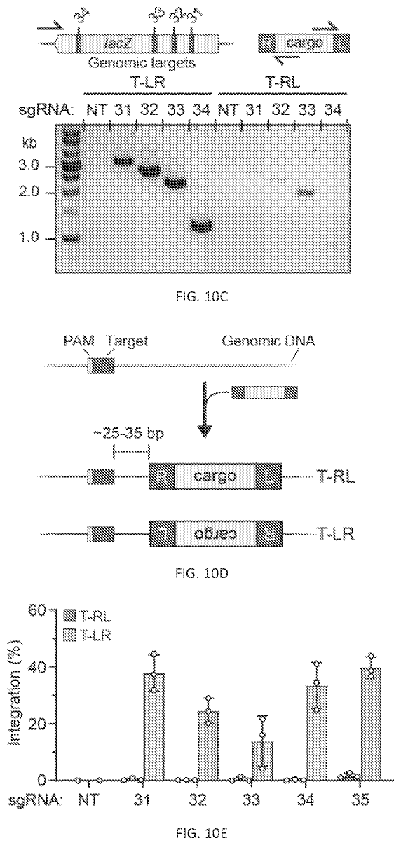

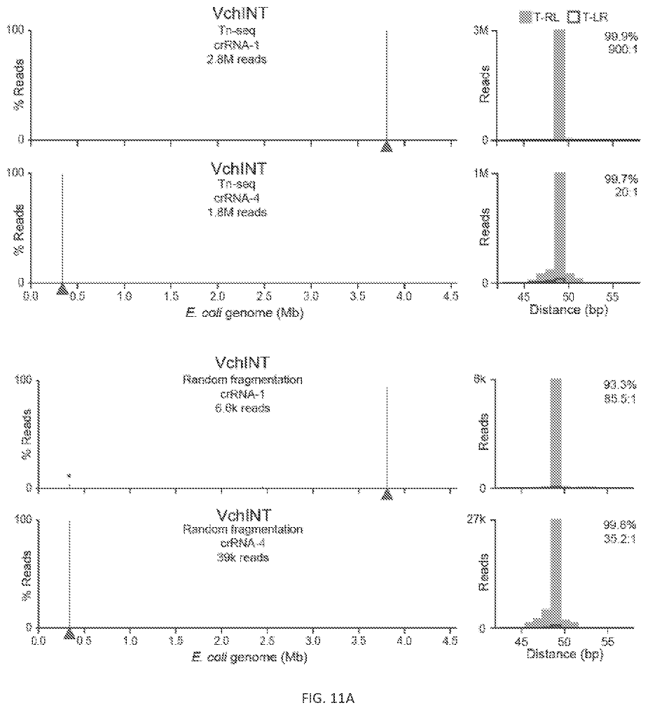

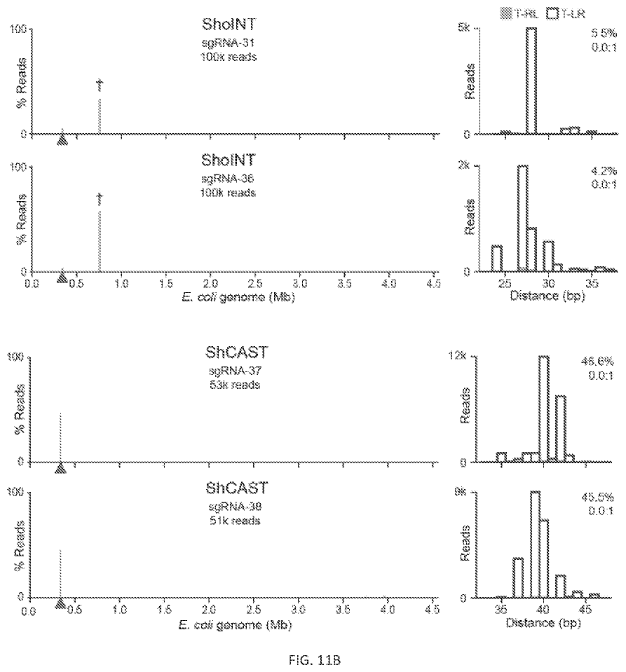

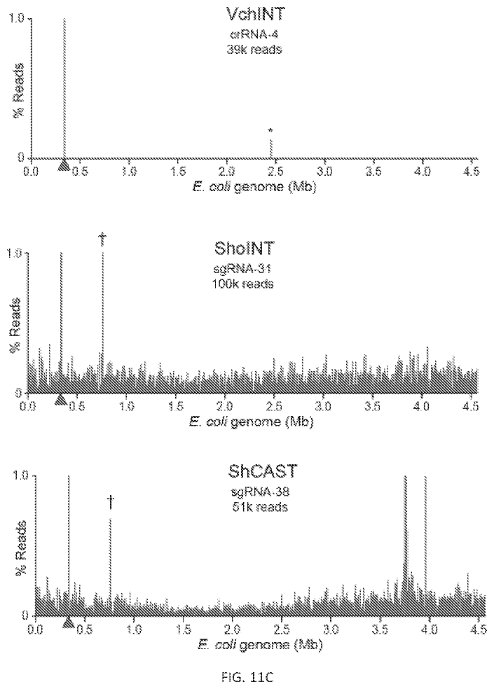

The simultaneous presence of a genomically integrated mini-Tn and distinct plasmid-borne mini-Tn produces an interesting scenario in which the transposase machinery can theoretically employ either DNA molecule as the donor substrate for integration (FIG. 10A). Using cargo-specific primers, new insertions at target−1 were indeed a heterogeneous mixture of both mini-Tn donors, although the higher-copy plasmid source was heavily preferred (FIG. 10A). To further investigate intramolecular transposition events, the clonally integrated strain was transformed with a plasmid encoding the protein-RNA machinery without donor DNA, and monitored re-mobilization of the pre-existing mini-Tn from target-4 to target-1. Integration at target-1 was readily observed, but surprisingly, there was no PCR evidence of mini-Tn loss at target-4, despite the expectation that the transposon mobilizes through a cut-and-paste mechanism (FIG. 3B), suggesting that lesions resulting from donor DNA excision are rapidly resolved by HR, as has been observed with Tn747.

To avoid any low-level contamination between donor DNA molecules, the use of multiple RNA-guided transposases whose cognate transposon ends would be recognized orthogonally was explored. Guided by prior bioinformatic description and experimental validation of transposons encoding Type V-K CRISPR-Cas systems, a new INTEGRATE system derived from Scytonema hofmannii strain PCC 7110 (hereafter ShoINT, FIG. 10B) was developed; the protein components are 30-55% identical to the homologous system described by Strecker and colleagues (ShCAST), which derives from a distinct S. hofmannii strain (FIG. 19). ShoINT catalyzes RNA-guided DNA integration with 20-40% efficiency, and strongly favors integration in the T-LR orientation, albeit with detectable bidirectional integration at multiple target sites (FIGS. 5C-5E). Next, pEffector plasmids for the V. cholerae INTEGRATE system (VchNT) or ShoINT were combined with either its own cognate pDonor or pDonor from the other system, and each RNA-guided integrase was exclusively active on its respective mini-Tn substrate (FIG. 3C). With this knowledge, a new cargo was sequentially introduced at different locus (at target-1) using ShoINT, without any secondary mobilization of the pre-existing VchINT mini-Tn (at target-4) (FIG. 3D). This approach of using systems with transposon ends that are sufficiently distinct enables orthogonal and iterative integration events for distinct genetic payloads.

After developing an alternative, unbiased NGS approach to query genome-wide integration events, which does not require the MmeI restriction enzyme used in Tn-seq, the random fragmentation-based method was verified to return similar specificity information for VchINT (FIG. 11A). When the same method was applied to ShoINT and ShCAST, only ˜5-50% integration events were on-target, with substantial numbers of insertions distributed randomly across the genome (FIGS. 11B-11C).

### Example 3

### Single-Step Multiplex DNA Insertions Using INTEGRATE

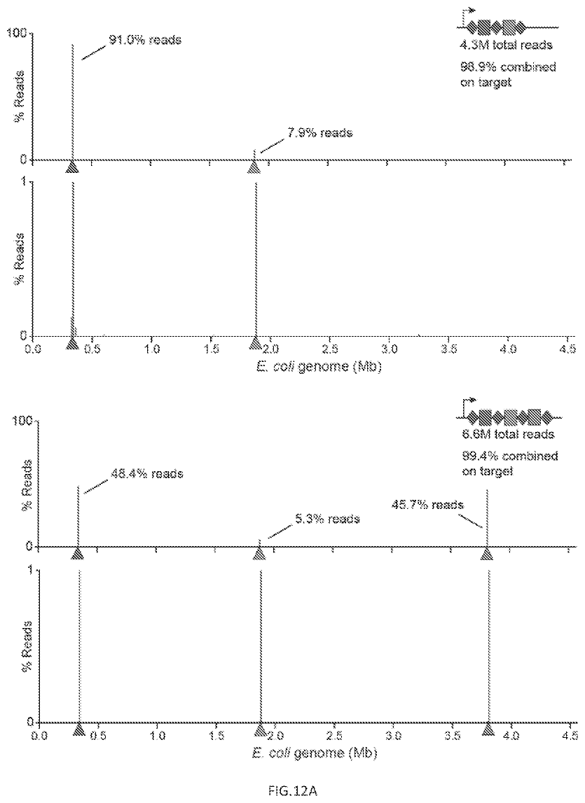

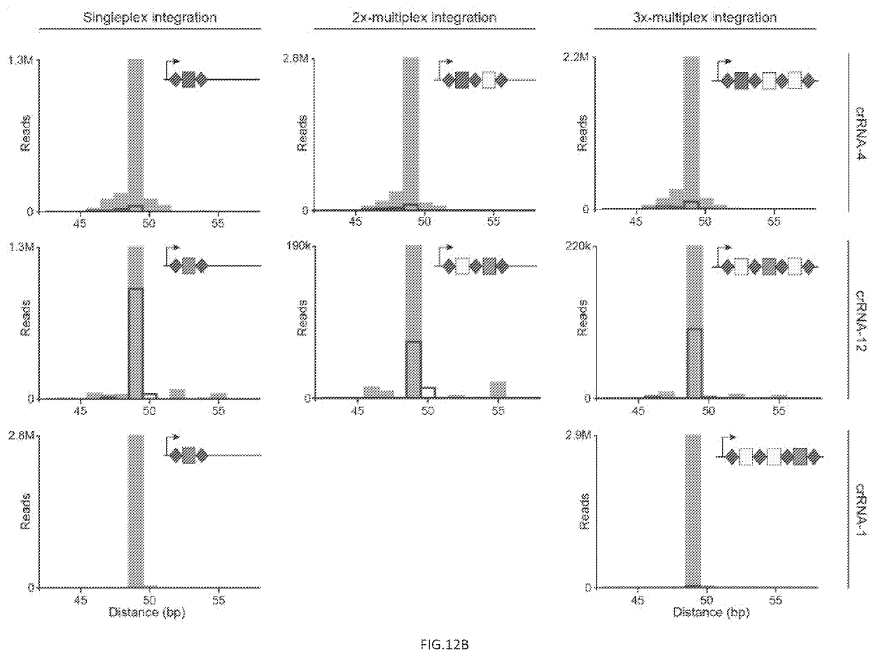

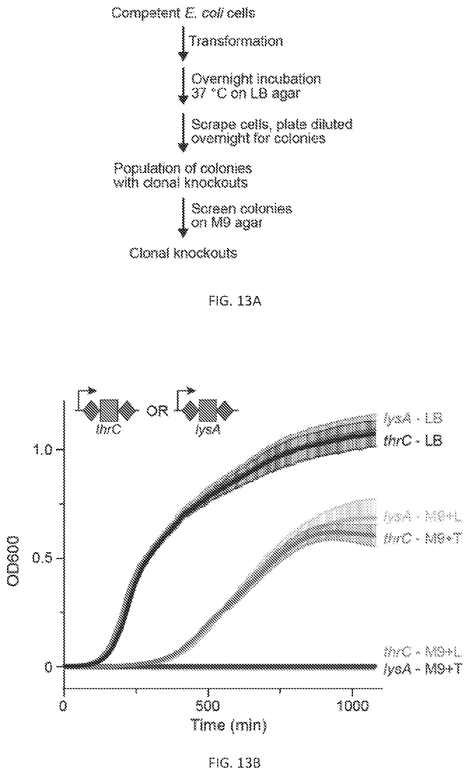

Multi-spacer CRISPR arrays provided a means to direct integration of the same cargo at multiple genomic targets simultaneously (FIG. 4A), which significantly reduces time and complexity for strain engineering projects requiring multi-copy integration. a series of multiple-spacer arrays into pSPIN were cloned, and the integration efficiency of a lacZ-specific crRNA was unchanged for two spacers and reduced by <2-fold for three spacers, depending on relative position, when cells were cultured at 37° C. (FIG. 4B). Tn-seq analyses with double- and triple-spacer arrays revealed >99% on-target transposition, with characteristics that were otherwise indistinguishable from single-plex insertions for each target site (FIGS. 4C and 12), and were further verified multiplex insertions by whole-genome SMRT sequencing of double- and triple-insertion clones.

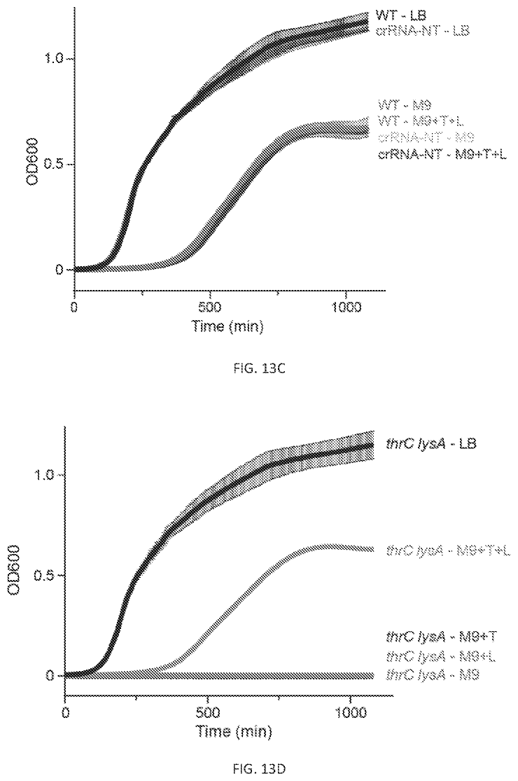

To further confirm that simultaneous insertions were indeed occurring within each individual chromosome rather than population-wide, an experiment to generate auxotrophic E. coli strains requiring both threonine and lysine for viability by insertionally inactivating thrC and lysA48 were designed (FIGS. 4D and 13A-13C). Double-knockout clones could be rapidly isolated after a single transformation step (FIG. 4E) and exhibited selective growth in M9 minimal media only when both threonine and lysine were supplemented (FIG. 4F). To probe the stability of integration-based knockouts, clones were cultured in rich media for five serial overnight passages without removing the expression plasmid and observed no change in the media requirements (FIG. 13D).

### Example 4

### One-Step Genomic Deletions Using INTEGRATE

Finally, the combined use of RNA-guided integrases with site-specific recombinases to mediate facile programmable, one-step genomic deletions was explored. Specifically, a LoxP site was inserted within the mini-Tn cargo and generated double-spacer CRISPR arrays to drive multiplex integration at two target sites. Subsequently, Cre recombinase was used to excise the chromosomal region within the LoxP sites, thus resulting in a precise deletion containing a single mini-Tn (FIG. 4G). CRISPR arrays were designed to produce 2.4-, 10-, and 20-kb deletions, which were confirmed via diagnostic PCR analysis and unbiased, whole-genome SMRT sequencing (FIGS. 4H-4I and 14).

### Example 5

### Broad Host-Range Activity of RNA-Guided Integrases

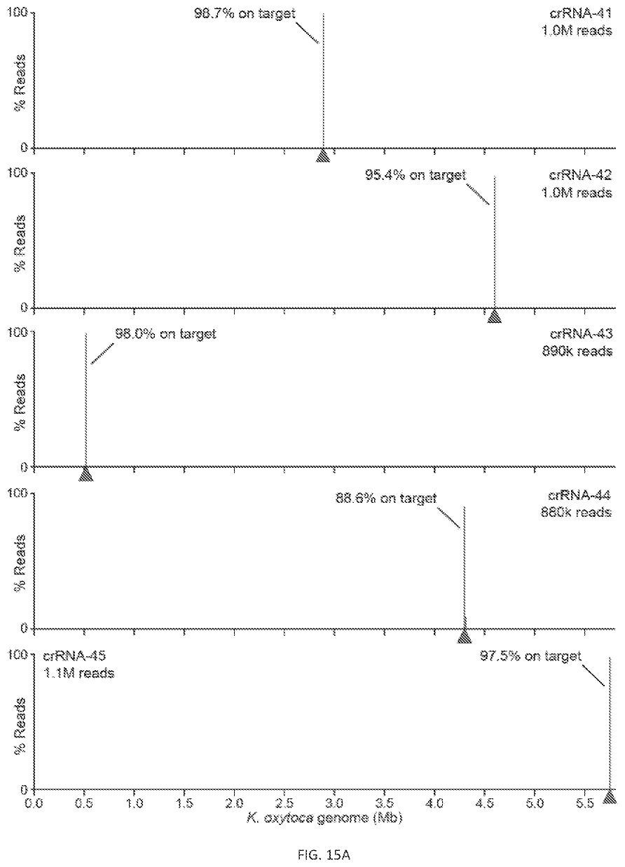

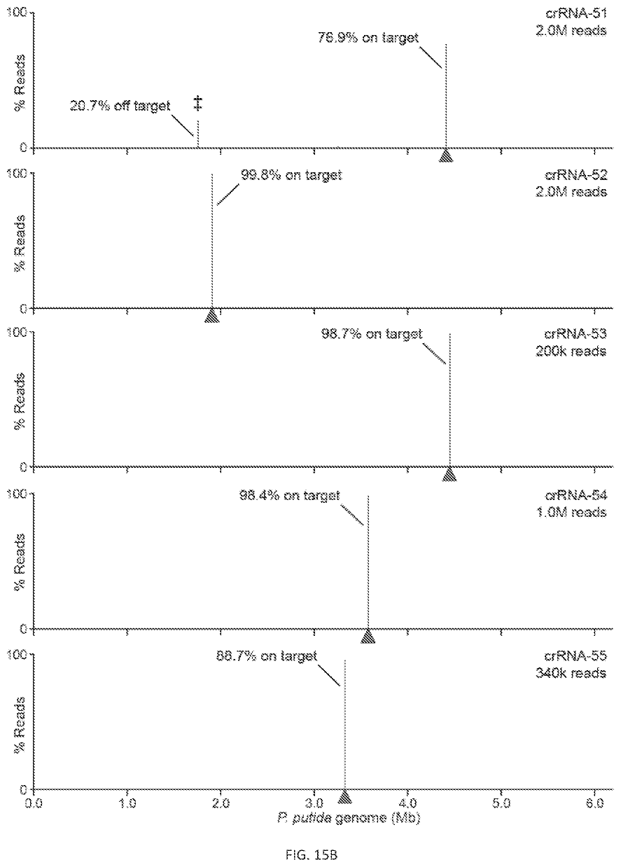

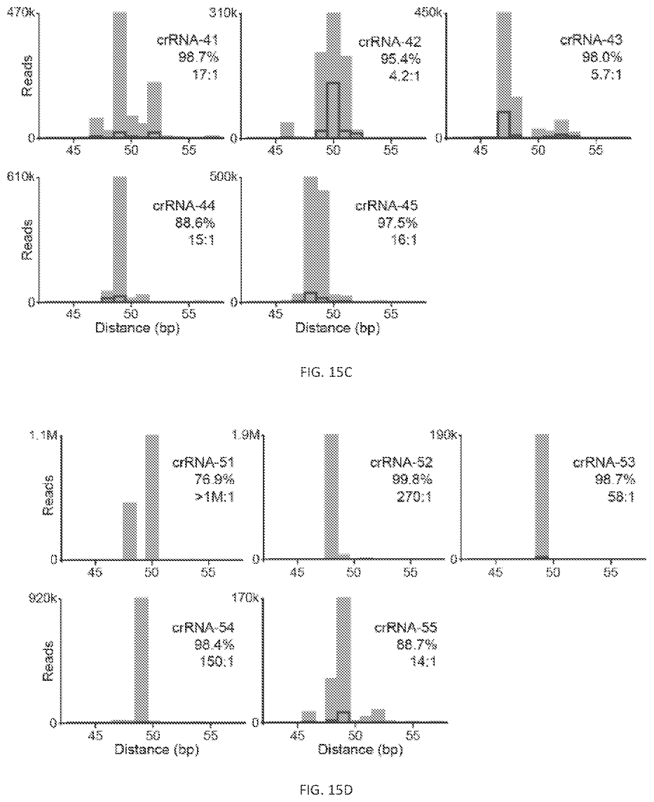

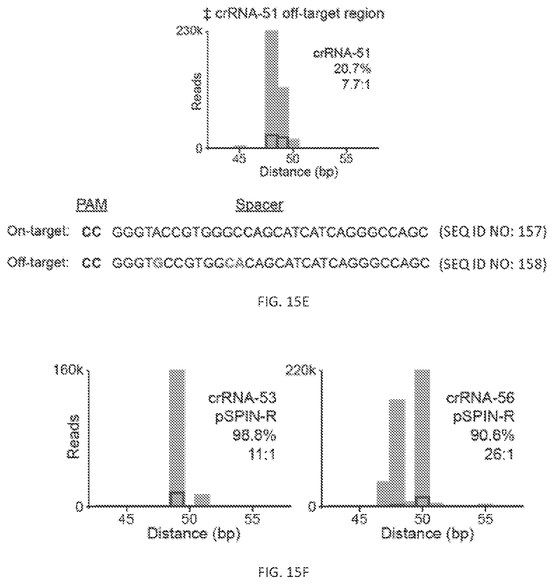

Mobile genetic elements, especially transposons, often ensure their evolutionary success by functioning robustly across a broad range of hosts, without a requirement for specific host factors. Given this expectation, as well as the efficiency with which the V. cholerae machinery directs RNA-guided transposition in E. coli, INTEGRATE activity was evaluated in other Gram-negative bacteria; Klebsiella oxytoca, a clinically relevant pathogen implicated in drug-resistant infections and emerging model organism for biorefinery, and Pseudomonas putida, an important bacterial platform for biotechnological and industrial applications (FIG. 5A). Using a pSPIN derivative driven by the constitutive J23119 promoter, four non-essential metabolic genes were targeted (xylA, galK, lacZ, and malK) and one antibiotic resistance gene (ampR) in K. oxytoca, as well as intergenic regions (upstream of PP_2928 and benR) or genes previously edited (nrC, nirD, bdhA, and PP_3889) in P. putida. For all 10 targets, highly-accurate RNA-guided DNA integration were observed by both PCR and Tn-seq, with similar integration distance and orientation bias profiles as seen in E. coli (FIGS. 5B-5C and 15A-15D). DNA insertions were virtually absent with a non-target crRNA, and on-target specificity was >95% on average, with the only outlier resulting from a prominent Cascade off-target binding site (FIG. 15E). Given the potential for INTEGRATE to exhibit off-target activity similar to canonical CRISPR-Cas systems, a computational tool was developed for guide RNA design and off-target prediction (FIG. 16).

Interestingly, for two of the P. putida crRNAs, a substantial enrichment of NGS reads mapping to pSPIN were observed, precisely 48-50 bp downstream of the spacer in the CRISPR array (FIG. 5D). Evidence of very low-level self-targeting was observed in all of the E. coli Tn-seq datasets, and the apparent abundance of self-targeting insertions for P. putida crRNAs targeting nicC and bhdA genes resulted from a fitness cost of the intended knockout and concomitant selective pressure to inactivate the pSPIN expression vector. When P. putida was transformed with modified pSPIN-R vectors encoding the exact same crRNAs but at the 3′ end of the fusion transcript, self-targeting was completely abrogated (FIGS. 5E and 15F). The utility of RNA-guided integrases for programmable genetic modifications exists across diverse bacterial species.

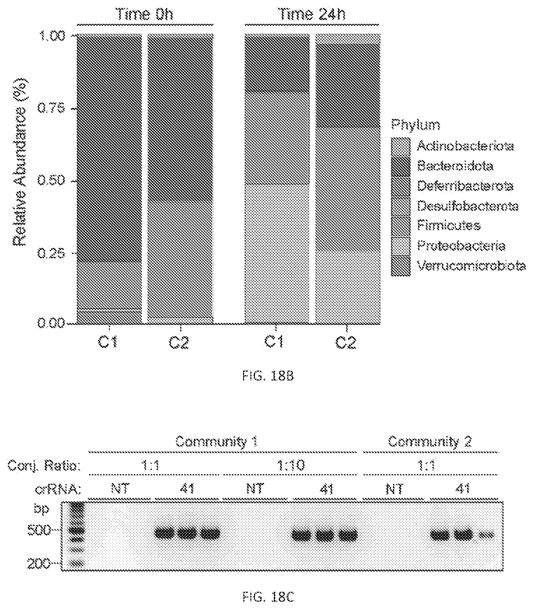

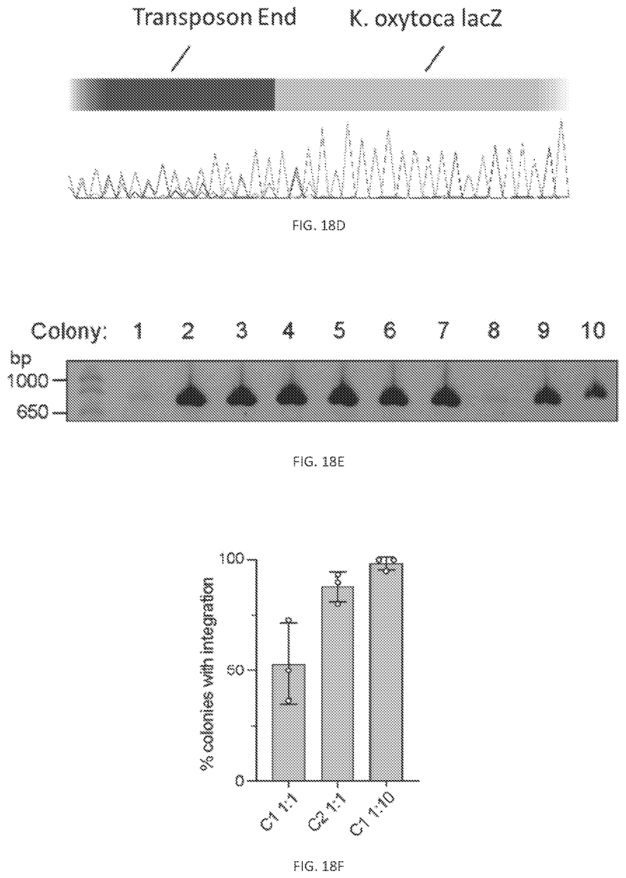

Bacterial conjugation was used to deliver pSPIN from a donor E. coli strain into a complex bacterial community derived from the mouse gut. The pSPIN construct was designed to specifically target the lacZ locus of a K. oxytoca strain added to the community. After isolating transconjugates, robust and high-efficiency RNA-guided transposition across distinct microbiome community sources, and with different donor-to-recipient ratios, was observed (FIG. 18). Thus, RNA-guided integrases have utility for programmable genetic modifications across diverse bacterial species and within complex microbiota.

Through systematic engineering steps, an optimized set of vectors was developed to leverage INTEGRATE for targeted DNA integration applications in diverse bacterial species, without the need for DSBs, HR, or cargo-specific marker selection. These streamlined constructs may be modified to generate user-specific guide RNAs and genetic cargos, and they catalyze highly accurate, large DNA insertions at ˜100% efficiency after a single transformation step. Moreover, by repurposing the natural CRISPR array, multi-spacer CRISPR arrays within the same seamless workflow, efficient multiplexing for simultaneous insertions or programmed genomic deletions was demonstrated by using INTEGRATE in combination with Cre-LoxP, within the same seamless workflow. The mini-Tn is compatible with any arbitrary target site, thus significantly reducing the complexity of the donor DNA and accelerating the experiment compared to HR, particularly for large-scale multiplex applications and metabolic engineering. This genetic engineering toolkit can be harnessed to generate large guide RNA libraries, which will enable high-throughput screening of rationally designed targeted DNA insertions that are not easily accessible with random transposase-based strategies. Libraries of multiplexed guide RNAs can enable synthetic lethality screening and investigations of pairwise interactions at the genome scale in bacteria. Furthermore, INTEGRATE can help advance existing strain engineering technologies, particularly those currently employing site-specific or non-specific transposases that could benefit from programmable site-specific insertions. The methods disclosed herein provide a process for increasing the efficacy of genetic manipulations.

In addition to its utility for strain engineering, INTEGRATE systems may be a particularly useful for species- and target-specific genetic manipulations in mixed bacterial communities and microbiome niches via the ability to broadly deliver all the necessary machinery on a single vector by conjugation. Using compact construct designs, a fully autonomous CRISPR-transposon was generated that was capable of high-efficiency integration. Similar constructs are mobilized on broad host-range conjugative plasmids, pre-programmed with multiple-spacer CRISPR arrays, to genetically modify desired bacterial species at user-defined target sites. The system and methods disclosed herein allow gene drive applications, such as inactivating antibiotic resistance genes or virulence factors and introducing genetic circuits and synthetic pathways in a targeted manner.

### Example 6

### Bacteroides vulgatus and Other Members of the Mammalian Gut Microbiome

The Bacteroides genus constitutes ˜30% of the total colonic bacteria, and this particular strain, alongside other Bacteroides strains that include thetaiotaomicron, fragilis, and ovatus are the most commonly encountered species in the human colon. Thus, this class of organisms represents a high-value target for genetic manipulations in the context of complex human-associated bacterial communities, or microbiomes, for therapeutics and basic research purposes. In particular, the ability to eliminate resident genes and/or insert new gene and biological functionalities, in a gene- and species-specific manner, opens up new opportunities for precision microbiome engineering.

Highly-precise targeted insertions can be robustly generated in Bacteroides vulgatus using the INTEGRATE CRISPR-transposon system from V. cholerae. Targeted insertions are characterized by a combination of junction PCR and Sanger sequencing to verify the insertion products, and next-generation sequencing (NGS) to verify the genome-wide specificity. Importantly, components for the INTEGRATE CRISPR-transposon system may be introduced in Bacteroides, and other members of the gut microbiome community, either via direct delivery of the expression vectors, or via conjugation from a donor strain containing the CRISPR-transposon system components.

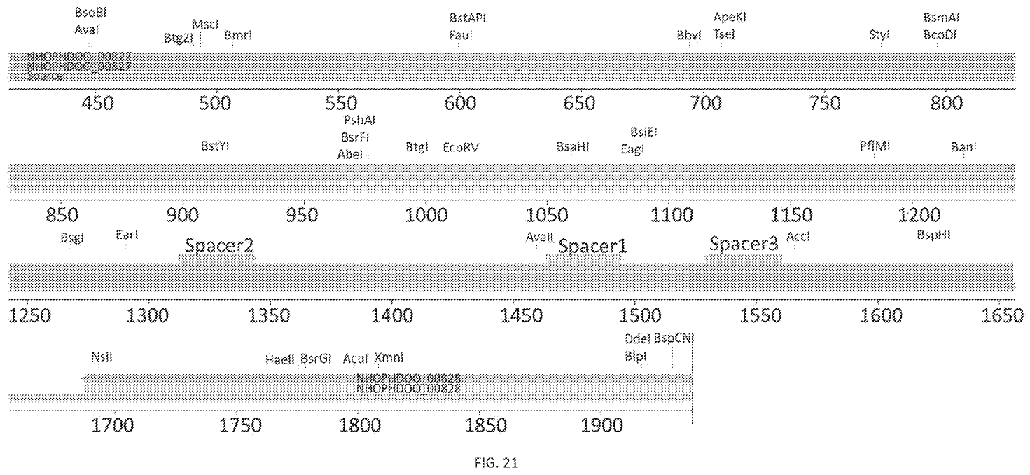

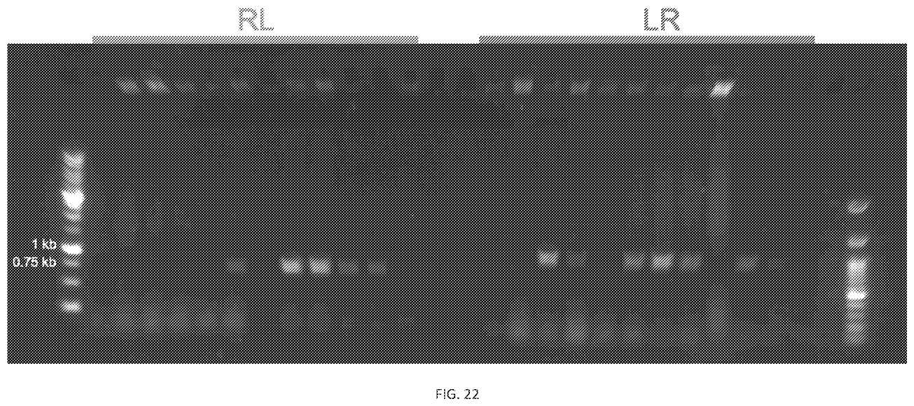

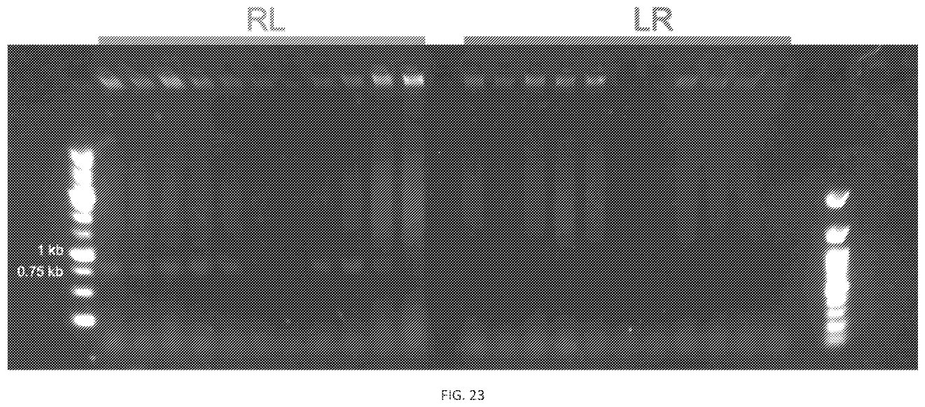

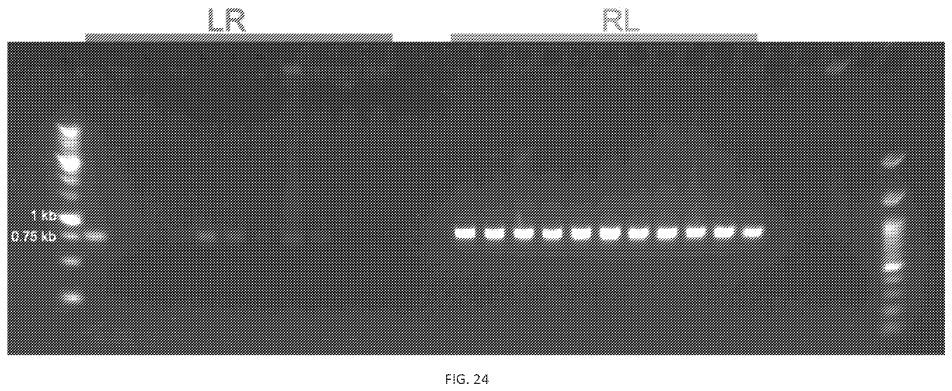

The pSPIN vectors, described herein, were adapted for Bacteroides through both codon optimization, inclusion of Bacteroides-specific ribosome binding sites (RBS), and inclusion of origins of replication that enable plasmid maintenance in Bacteroides. The pSPIN derivative vector also included an origin of transfer sequence, to enable conjugation from the S17 donor strain of E. coli, as described in Ronda et al. (Nat Methods 16, 167-170 (2019), incorporated herein by reference in its entirety), as well as drug markers that enable selection in Bacteroides vulgatus. The sequence of a representative entry-vector version of this Bacteroides-specific pSPIN vector, also known as pSL2130, is SEQ ID NO: 257. This vector contains BbsI restriction sites to facilitate new spacer cloning into the CRISPR array, upon selection of appropriate targeting sequences for new guide RNAs. Three spacers were chosen to introduce a site-specific insertion within the bile salt hydrolase (BSH) gene of Bacteroides vulgatus; sequences for these spacers are shown in SEQ ID NOs: 258-260, and the relative position of these (proto)spacers within the BSH gene is depicted in FIG. 21.

Bacteroides-specific pSPIN vectors containing the guide RNA of interest were introduced into the E. coli S17 donor strain through standard transformation procedures. Subsequently, conjugation reactions were prepared with Bacteroides vulgatus under anaerobic conditions, following standard procedures (See. Ronda et al., Nat Methods 16, 167-170 (2019), incorporated herein by reference in its entirety), in order to facilitate transfer of the pSPIN vector from the donor E. coli strain to the recipient B. vulgatus strain. Following initial outgrowth on non-selective media, cells were replated on media that selects for drug resistance (encoded by pSPIN) and kills the donor strain (which is engineered to be auxotrophic). After sufficient culturing, cells were harvested and analyzed for targeted DNA integration using phenotypic assays, standard PCR, qPCR, NGS, and/or whole-genome sequencing approaches.

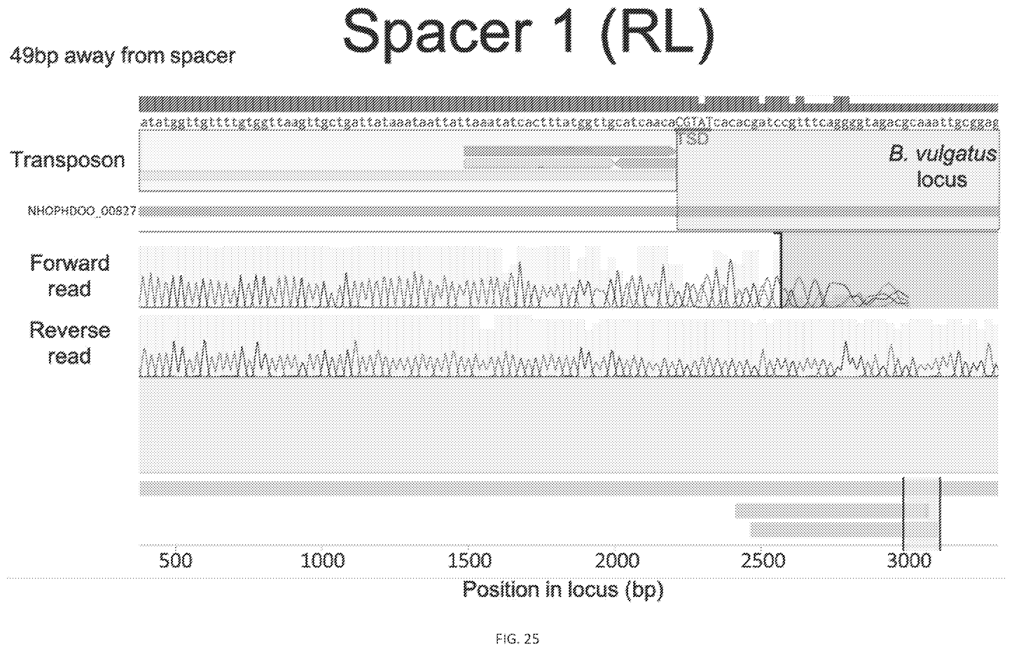

After performing conjugation reactions with pSPIN vectors encoding guide RNAs with spacers 1, 2, and 3, each of which targets within the same BSH gene, cells were selected on drug-containing media, colonies were removed and lysed, and then the lysate was subjected to junction PCR analysis. In this PCR, a transposon and/or transposon cargo-specific primer was compared with a genome-specific primer, such that amplification products were only generated in the event of targeted integration proximal to the target site matching the guide RNA. In experiments with all three spacers, specific junction PCR product bands were generated at the expected site (FIGS. 22-24), indicating successful RNA-guided DNA integration in Bacteroides vulgatus. As has been observed previously, integration products can occur in one of two orientations, in which either the transposon ‘right’ (R) end is integrated proximally to the target site (denoted tRL or simply ‘RL’ product), or in which the transposon ‘left’ (L) end is integrated proximally to the target site (denoted tLR, or simply ‘LR’ product). As is seen in FIGS. 22-24, all three spacers had the capacity to generate insertions in either orientation, though in some cases one orientation was clearly biased/preferred over the other.

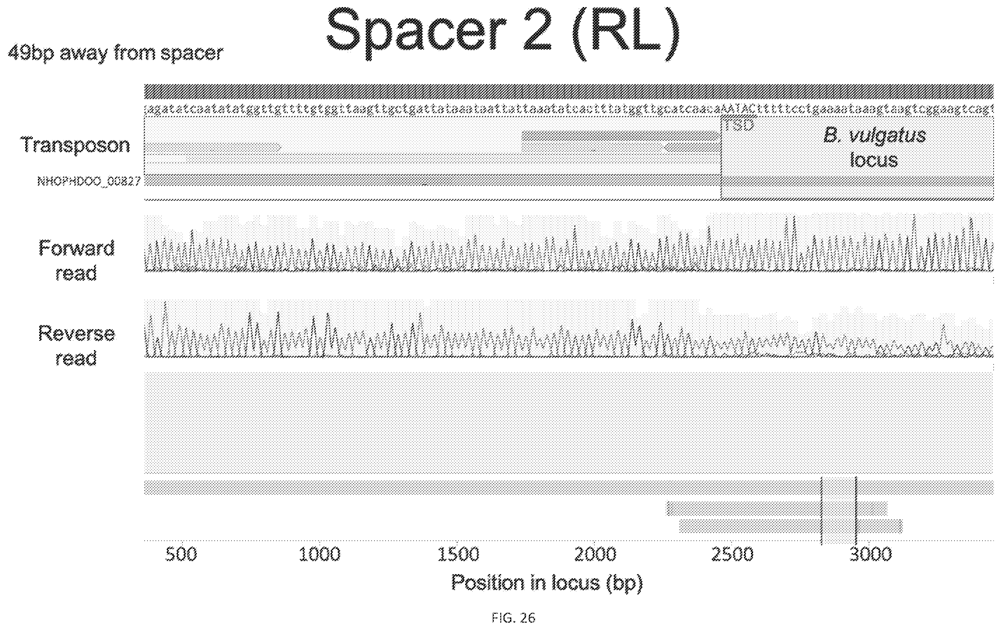

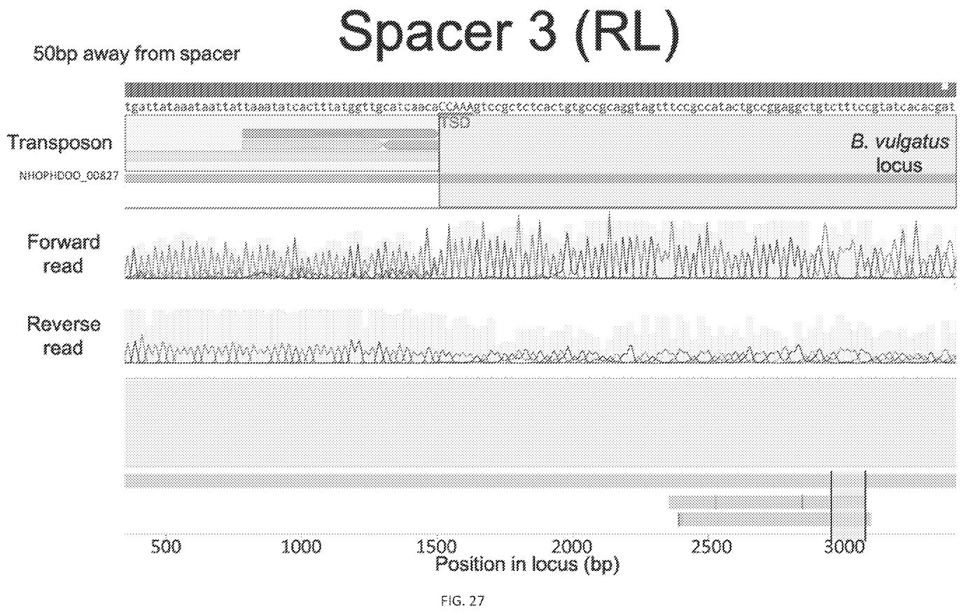

To further confirm that targeted insertions were indeed RNA-guided and “on-target,” PCR products were analyzed by Sanger sequencing. These analyses indicated that, in all cases, the transposon was inserted precisely 49-50 bp downstream of the target site. Sanger sequencing chromatograms for these analyses, aligned to the reference genome containing the insertion product, are shown in FIGS. 25-27.

The same pSPIN designs can be adapted for other bacterial species, genus, families, orders, classes, or phyla, that populate the human microbiome. The adaptation process may include optimization of various gene parts for the biology of the target organism(s), including, but not limited to, promoter elements, codon usages, ribosome binding sites, transcriptional terminators, origins of replication, conjugation machineries, and resistance markers. To enable multiplexed targeting of multiple genes within the same species, or multiple genes across multiple species, the CRISPR array is expanded to encode multiple guide RNAs, such that the CRISPR and transposase machineries can target a range of genomic sites.

Similar conjugation strategies may be applied to deliver the CRISPR-transposon system (also known as INTEGRATE) into multiple recipient organisms in a single step, in the case where a donor strain is mixed with a complex bacterial community containing more than one recipient strain. Subsequent analyses may be performed on the bulk population, or on isolated clones. In some embodiments, the entire bacterial community containing the targeted insertions are then used in downstream steps, whether for microbiome transplantation into animal or human subjects, or other downstream applications. In some embodiments, the recipient community is derived from stool samples from an animal model or from a human patient (known as a fecal microbiome or fecal bacterial community). In other embodiments, the recipient community may derive from other microbiome environments, including but not limited to other parts of the human body, soil samples, or other ecological environments.

The transposon can be programmed with a wide array of various cargo genes, or payloads, in which one or more biologically functionalities are encoded. Additionally, genes may be included that provide enhanced fitness to the recipient organism, such that insertion events are enriched without the need for drug selection.

It is understood that the foregoing detailed description and accompanying examples are merely illustrative and are not to be taken as limitations upon the scope of the disclosure, which is defined solely by the appended claims and their equivalents.

All publications and patents mentioned in the above specification are herein incorporated by reference as if expressly set forth herein. Various changes and modifications to the disclosed embodiments will be apparent to those skilled in the art and may be made without departing from the spirit and scope thereof.

What is claimed is:

1. A system for targeted nucleic acid deletions comprising:
an engineered Clustered Regularly Interspaced Short Palindromic Repeats (CRISPR)-CRISPR associated (Cas) (CRISPR-Cas) system, and/or one or more vectors encoding the engineered CRISPR-Cas system, wherein the engineered CRISPR-Cas system comprises: (a) at least one Cas protein, and (b) a pair of guide RNAs (gRNAs), wherein the pair of gRNAs is configured to hybridize to target sites flanking a nucleic acid sequence for deletion;
an engineered transposon system, and/or one or more vectors encoding the engineered transposon system;
a recombinase, or catalytic domain thereof, and/or one or more vectors encoding a recombinase, or catalytic domain thereof; and
at least one donor nucleic acid to be integrated, wherein the donor nucleic acid comprises a recognition site for the recombinase flanked by at least one transposon end sequence.

2. The system of claim 1, wherein the engineered CRISPR-Cas system and the engineered transposon system are on the same or different vector(s).

3. The system of claim 1 or 2, wherein the recombinase, or catalytic domain thereof, is on the same or different vector(s) from the engineered CRISPR-Cas system and/or the engineered transposon system.

4. The system of any of claims 1-3, wherein the recombinase, or catalytic domain thereof, comprises a tyrosine recombinase.

5. The system of any of claims 1-4, wherein the recombinase comprises Cre recombinase, a mutant, variant, or catalytic domain thereof and the recognition site comprises a lox site or variant thereof.

6. The system of any of claims 1-5, wherein the recombinase comprises flippase (FLP) recombinase, a mutant, variant, or catalytic domain thereof and the recognition site comprises a flippase recognition target (FRT) site or variant thereof.

7. The system of any of claims 1-6, wherein the recombinase, or catalytic domain thereof, comprises a serine recombinase.

8. The system of any of claims 1-7, wherein the recombinase comprises TniR resolvase, a mutant, variant, or catalytic domain thereof and the recognition site comprises a resolution (res) sequence or variant thereof.

9. The system of any of claims 1-8, wherein the recombinase comprises a Tn3-like resolvase, mutant, variant, or catalytic domain thereof and the recognition site comprises a resolution (res) sequence or variant thereof.

10. The system of any of claims 1-9, wherein the engineered CRISPR-Cas system comprises a Type V system or a Type I system.

11. The system of any of claims 1-10, wherein the engineered CRISPR-Cas system comprises Cas12k.

12. The system of any of claims 1-11, wherein the engineered CRISPR-Cas system comprises Cas5, Cas6, Cas7, Cas8, or a combination thereof.

13. The system of any of claim 1-12, wherein the engineered transposon system comprises TnsA, TnsB, TnsC, or a combination thereof.

14. The system of any of claims 1-13, wherein the engineered transposon system comprises TniQ.

15. The system of any of claims 1-14, wherein the donor nucleic acid comprises a human nucleic acid sequence.

16. The system of any of claims 1-15, wherein the donor nucleic acid further comprises a cargo nucleic acid.

17. The system of claim 16, wherein the cargo nucleic acid comprises the recognition site for the recombinase.

18. The system of any of claims 1-17, further comprising a target nucleic acid comprising the nucleic acid sequence for deletion.

19. The system of any of claims 1-18, wherein at least one of the pair of guide RNAs is a non-naturally occurring gRNA.

20. The system of any of claim 1-19, wherein the system is a cell-free system.

21. A cell comprising the system of any one of claims 1-19.

22. The cell of claim 21, wherein the cell is a eukaryotic cell.

23. The cell of claim 21 or 22, wherein the nucleic acid sequence for deletion is an endogenous nucleic acid.

24. The cell of any of claims 21-23, wherein the nucleic acid sequence for deletion is genomic DNA.

25. A method for deleting a nucleic acid sequence from a target nucleic acid, wherein the method comprises contacting the target nucleic acid with the system of any one of claims 1-19.

26. The method of claim 25, wherein the target nucleic acid is in a cell and contacting the target nucleic acid comprises introducing into the cell.

27. The method of claim 26, wherein the recombinase, or catalytic domain thereof, and/or one or more vectors encoding a recombinase, or catalytic domain thereof is introduced to the cell after the introduction of the engineered CRISPR-Cas system, the engineered transposon system, and the at least one donor nucleic acid.

28. The method of claim 26 or 27, wherein the introducing into the cell comprises administering to a subject.

29. The method of claim 28, wherein the administering comprises intravenous administration.

30. A method for inactivating a gene of interest, the method comprising introducing into one or more cells the system of any one of claims 1-19, wherein the nucleic acid sequence for deletion comprises at least a portion of the gene of interest.

31. The method of claim 30, wherein the one or more cells comprises microbial cells.

32. The method of claim 30, wherein the one or more cells comprises plant cells.

33. The method of claim 30, wherein the one or more cells comprises animal cells.

34. The method of any of claims 30-33, wherein the gene of interest comprises an antibiotic resistance gene, a virulence gene, or a metabolic gene.

35. Use of the system of any one of claims 1-19 for the deletion of a nucleic acid sequence from a target nucleic acid.

36. The use of claim 35, wherein the target nucleic acid sequence is in a cell.

37. The use of claim 36, wherein the cell comprises a eukaryotic cell.

38. The use of any of claims 35-37, wherein the nucleic acid sequence comprises at least a portion of a gene.

39. A method for genetically modifying diverse bacterial communities comprising:
contacting a recipient bacterial community with donor bacteria, the donor bacteria comprising a vector encoding:
an engineered Clustered Regularly Interspaced Short Palindromic Repeats (CRISPR)-CRISPR associated (Cas) (CRISPR-Cas) system, wherein the engineered CRISPR-Cas system comprises: (a) at least one Cas protein, and (b) at least one guide RNA (gRNA);
an engineered transposon system; and
at least one donor nucleic acid to be integrated comprising at least one transposon end sequence.

40. The method of claim 39, wherein the engineered CRISPR-Cas system comprises a Type V system or a Type I system.

41. The method of claim 39 or 40, wherein the engineered CRISPR-Cas system comprises Cas12k.

42. The method of any of claims 39-41, wherein the engineered CRISPR-Cas system comprises Cas5, Cas6, Cas7, Cas8, or a combination thereof.

43. The method of claim 42, wherein the engineered CRISPR-Cas system comprises a Cas8-Cas5 fusion protein.

44. The method of any of claims 39-43, wherein the engineered transposon system is derived from a Tn7 transposon system.

45. The method of any of claims 39-44, wherein the engineered transposon system comprises TnsA, TnsB, TnsC, or a combination thereof.

46. The method of any of claims 39-45, wherein the engineered transposon system comprises TniQ.

47. The system of any of claims 39-46, wherein the donor nucleic acid further comprises a cargo nucleic acid flanked by the at least one transposon end sequence.

48. The method of any of claims 39-47, wherein the vector further encodes a recombinase, or a catalytic domain thereof, and the at least one donor nucleic acid further comprises a recognition site for the recombinase.

49. The method of claim 48, wherein the cargo nucleic acid comprises the recognition site for the recombinase.

50. The method of claim 48 or 49, wherein the recombinase comprises Cre recombinase, a mutant, variant, or catalytic domain thereof and the recognition site comprises a lox site or variant thereof.

51. The method of any of claims 48-50, wherein the recombinase comprises flippase (FLP) recombinase, a mutant, variant, or catalytic domain thereof and the recognition site comprises a flippase recognition target (FRT) site or variant thereof.

52. The method of any of claims 48-51, wherein the recombinase, or catalytic domain thereof, comprises a serine recombinase.

53. The method of any of claims 48-52, wherein the recombinase comprises TniR resolvase, a mutant, variant, or catalytic domain thereof and the recognition site comprises a resolution (res) sequence or variant thereof.

54. The method of any of claims 48-53, wherein the recombinase comprises a Tn3-like resolvase, mutant, variant, or catalytic domain thereof and the recognition site comprises a resolution (res) sequence or variant thereof.

55. The method of any of claims 48-54, wherein the engineered CRISPR-Cas system comprises a pair of guide RNAs (gRNAs), wherein the pair of gRNAs is configured to hybridize to target sites flanking a nucleic acid sequence for deletion.

56. The method of claim 55, wherein the nucleic acid sequence for deletion comprises a genomic nucleic acid sequence endogenous to the recipient bacterial community.

57. The method of any of claims 39-56, wherein the engineered CRISPR-Cas system, the engineered transposon system, the recombinase, or a combination thereof are encoded within the at least one donor nucleic acid.

58. The method of any of claims 39-57, wherein the engineered CRISPR-Cas system, the engineered transposon system, the recombinase, or a combination thereof are encoded within the cargo nucleic acid.

59. The method of any of claims 39-58, wherein the recipient bacterial community is isolated from fecal matter.

60. The method of any of claims 39-59, wherein the recipient bacterial community comprises gut bacteria.

61. The method of any of claims 39-60, wherein the vector is a conjugative plasmid.
